(19) United States
(12) Patent Application Publication
(10) Pub. No.: US 2018/0015051 A1
(43) Pub. Date: Jan. 18, 2018

(54) MODULATORS OF ALPHA-DICARBONYL DETOXIFICATION AND THEIR USE FOR THE TREATMENT OF DIABETIC PATHOLOGIES

(71) Applicant: Buck Institute for Research on Aging, Novato, CA (US)

(72) Inventors: PANKAJ KAPAHI, Kensington, CA (US); NEELANJAN BOSE, Burlingame, CA (US); JYOTISKA CHAUDHURI, Petaluma, CA (US)

(21) Appl. No.: 15/648,318

(22) Filed: Jul. 12, 2017

Related U.S. Application Data
(60) Provisional application No. 62/362,420, filed on Jul. 14, 2016.

(51) Int. Cl. A61K 31/015; A61K 31/404; A61K 31/192; C07C 59/54; A61K 31/05

(52) U.S. Cl. CPC A61K 31/015; C07C 59/54; A61K 31/05; A61K 31/192; A61K 31/404

(57) ABSTRACT

In various embodiments compositions and methods are provided for ameliorating a pathology characterized by elevated α-dicarbonyl compounds or prophylactically slowing or stopping the onset of said pathology in a mammal. In certain embodiments the method comprises administering to the mammal an agent that activates TRPA1 in an amount sufficient to activate TRPA1, and/or to ameliorate one or more symptoms of the pathology (e.g., diabetes or a complication thereof), and/or to slow or stop the onset of the pathology, and/or to lower the level of α-dicarbonyl compounds in the mammal.

### CROSS-REFERENCE TO RELATED APPLICATIONS

This application claims benefit of and priority to U.S. Ser. No. 62/362,420, filed on Jul. 14, 2017, which is incorporated herein by reference in its entirety for all purposes.

### STATEMENT OF GOVERNMENTAL SUPPORT

This invention was made with government support under Grant Nos. R01AG038688, AG038012, AG045835, and R01AG048072 awarded by the National Institutes of Health. The Government has certain rights in this invention.

### BACKGROUND

Patients suffering from long-term diabetes mellitus, a disorder characterized by systemic hyperglycemia, often develop several metabolic and biochemical aberrations, most importantly elevation of a series of highly reactive α-dicarbonyl compounds (α-DCs, e.g., glyoxal/GO, methylglyoxal/MGO, and 3-deoxyglucosone/3DG) (Thornalley (1994) Amino Acids, 6: 15-23). These α-DCs are unavoidable byproducts of anaerobic glycolysis and lipid peroxidation and react indiscriminately with proteins, lipids, and DNA to yield a heterogeneous group of molecules, collectively called advanced glycation end products (AGEs) (Peppa and Vlassara (2005) Hormones, 4: 28-37). AGE formation renders irreversible damage to these biological macromolecules, altering their structural and functional integrity (Brownlee, 1995; Monnier et al., 2005). A large body of evidence has linked accelerated AGE formation via the in vivo accumulation of reactive a-DCs, specifically MGO in long-term diabetics, to the pathogenesis of many forms of diabetic complications. These include peripheral neuropathy, neurodegenerative conditions, cardiomyopathy, nephropathy, retinopathy, microvascular damage, and early mortality (Brownlee (1995) Ann. Rev. Med., 46: 223-234; Monnier et al. (2005) Ann. N.Y. Acad. Sci., 1043: 567-581; Peppa and Vlassara (2005) Hormones, 4: 28-37; Singh et al. (2014) Off. J. Korean Physiological Society and the Korean Society Pharmacol. 18: 1-14). Given these deleterious physiological effects of α-DC stress, cellular detoxification of these metabolites is highly relevant in delaying the progression of diabetic complications. The evolutionarily conserved glutathione-dependent glyoxalase system, comprised of glyoxalase I and II (human GLO1 and 2), is believed to be primarily responsible for a-DC detoxification and has recently garnered significant scientific interest in the context of diabetic complications (FIG. 2, panel A) (Sousa et al. (2013) Biochem. J. 453: 1-15).

Projections by the International Diabetes Federation (Islam et al. (2013) J. Diabetes Res. Art Id: 593204) have created an enormous urgency for the discovery of novel therapeutics and thus an immediate necessity for developing model systems that allow rapid assessment of the consequences of in vivo a-DC accumulation. In vertebrate models such as mice, it is generally difficult to perform causation studies (Robertson et al. (2011) J. Gerontol. Series A, Biol. Sci. Med. Sci., 66: 279-286) due to their comparatively long lifespan and the time it takes to develop the manifestations of α-DC stress.

### SUMMARY

Various embodiments contemplated herein may include, but need not be limited to, one or more of the following:

Various embodiments contemplated herein may include, but need not be limited to, one or more of the following:

Embodiment 1: A method for the treatment or prophylaxis of diabetes in a mammal, said method comprising: administering to a mammal identified as having diabetes or pre-diabetes an agent that activates TRPA1 in an amount sufficient to ameliorate one or more symptoms of diabetes or pre-diabetes.

Embodiment 2: The method of embodiment 1, wherein said amount sufficient to ameliorate one or more symptoms of diabetes or pre-diabetes is an amount sufficient to ameliorate a complication of diabetes selected from the group consisting of diabetic neuropathy, cardiomyopathy, nephropathy, retinopathy, microvascular damage, and early mortality.

Embodiment 3: A method of ameliorating a pathology characterized by elevated α-dicarbonyl compounds and advanced glycation endproducts or prophylactically slowing or stopping the onset of said pathology in a mammal, said method comprising: administering to said mammal an agent that activates TRPA1 in an amount sufficient to activate TRPA1 and/or to ameliorate one or more symptoms of said pathology, and/or to slow or stop the onset of said pathology, and/or to lower the level of dicarbonyl compounds in said mammal.

Embodiment 4: The method of embodiment 3, wherein said pathology is selected from the group consisting of Diabetes, Alzheimer's disease, Parkinson's disease, ATTR amyloidosis, cataract formation, stroke, and cardiovascular disease.

Embodiment 5: The method of embodiment 3, wherein said pathology is diabetes.

Embodiment 6: The method of embodiment 3, wherein said pathology is hyperglycemia.

Embodiment 7: A method of reducing the levels of α-dicarbonyl compounds and advanced glycation endproducts in a mammal, said method comprising: administering to said mammal an agent that activates TRPA1 in an amount sufficient to lower the level of α-dicarbonyl compounds and advanced glycation endproducts in said mammal.

Embodiment 8: A method of reducing a method of reducing the amount of, or slowing or stopping the formation and/or accumulation of, advanced glycation endproducts in a mammal, said method comprising: administering to said mammal an agent that activates TRPA1 in an amount sufficient to slow or stop the accumulation of advanced glycation endproducts in said mammal.

Embodiment 9: The method according to any one of embodiments 1-8, wherein said mammal is a mammal identified as having elevated triglycerides.

Embodiment 10: The method according to any one of embodiments 1-8, wherein said mammal is a mammal diagnosed as pre-diabetic.

Embodiment 11: The method according to any one of embodiments 1-8, wherein said mammal is a mammal diagnosed as having diabetes.

Embodiment 12: The method according to any one of embodiments 1-10 , wherein said method produces a reduction in one or more advanced glycation endproducts.

Embodiment 13: The method of embodiment 12, wherein said method produces a reduction in, or slows the accumulation of, glyoxal/GO.

Embodiment 14: The method according to any one of embodiments 12-13, wherein said method produces a reduction in, or slows the accumulation of, methylglyoxal/MGO.

Embodiment 15: The method according to any one of embodiments 12-14, wherein said method produces a reduction in, or slows the accumulation of 3-deoxyglucosone/3DG.

Embodiment 16: The method according to any one of embodiments 1-15, wherein said mammal is a human.

Embodiment 17: The method according to any one of embodiments 1-15, wherein said mammal is a non-human mammal.

Embodiment 18: The method according to any one of embodiments 1-17, wherein said TRPA1 activator is not a natural product other than podocarpic acid and/or a podocarpic acid derivative.

Embodiment 19: The method according to any one of embodiments 1-18, wherein method does not involve administering an agent selected from the group consisting of vitamin C, benfotiamine, pyridoxamine, alpha-lipoic acid, taurine, pimagedine, aspirin, carnosine, metformin, pioglitazone, pentoxifylline, resveratrol, and curcumin.

Embodiment 20: The method according to any one of embodiments 1-17, wherein said TRPA1 activator comprises podocarpic acid or an analog and/or derivative thereof or a pharmaceutically acceptable salt of said podocarpic acid or analog and/or derivative thereof.

Embodiment 21: The method of embodiment 20, wherein said podocarpic analog or derivative comprises podocarpanol or a pharmaceutically acceptable salt thereof.

Embodiment 22: The method of embodiment 20, wherein said podocarpic analog or derivative comprises a compound selected from the compounds shown in Table 1, Table 2, or Table 3 or a pharmaceutically acceptable salt thereof.

Embodiment 23: The method according to any one of embodiments 1-17, wherein said TRPA1 activator comprises an indolinone compound according to formula I or a pharmaceutically acceptable salt thereof.

Embodiment 24: The method of embodiment 21, wherein said indolinone compound is selected from the group consisting of is (2E)-[1-(cyclohexylmethyl)-2-oxo-1,2-dihydro-3H-indol-3-ylidene]acetic acid, (2E)-(1-benzyl-5-fluoro-2-oxo-1,2-dihydro-3H-indol-3-ylidene)acetic acid, (2E)-(1-benzyl-7-fluoro-2-oxo-1,2-dihydro-3H-indol-3-ylidene)acetic acid, (2E)-[-(cyclopentylmethyl)-2-oxo-1,2-dihydro-3H-indol-3-ylidene]acetic acid, (2E)-(7-fluoro-1-isobutyl-2-oxo-1,2-dihydro-3H-indol-3-ylidene)- acetic acid, (2E)-[-(cyclopentylmethyl)-7-fluoro-2-oxo-1,2-dihydro-3H-indol-3-ylidene]acetic acid, (2E)-(7-chloro-1-isobutyl-2-oxo-1,2-dihydro-3H-indol-3-ylidene)acetic acid, (2E)-[1-(cyclobutylmethyl)-7-fluoro-2-oxo-1,2-dihydro-3H-indol-3-ylidene]acetic acid, (2E)-[-(cyclopropylmethyl)-7-fluoro-2-oxo-1,2-dihydro-3H-indol-3-ylidene]acetic acid, (2E)-2-[1-(cyclopentylmethyl)-7-fluoro-2-oxo-1,2-dihydro-3H-indol-3-ylidene]-N,N-dimethylacetamide, (3E)-1-(2-ethyl butyl)-7-fluoro-3-(2-morpholin-4-yl-2-oxoethylidene)-1,3-dihydro-2H-indol-2-one, (2E)-{7-fluoro-1-[(2S)-2-methylbutyl]-2-oxo-1,2-dihydro-3H-indol-3-ylidene}acetic acid, (2E)-[7-fluoro-1-(3-methylbutyl)-2-oxo-1,2-dihydro-3H-indol-3-ylidene]acetic acid, (2E)-[1-(cyclohexylmethyl)-2-oxo-1,2-dihydro-3H-indol-3-ylidene]-N,N-dimethylacetamide, (2E)-2-[1-(cyclopentylmethyl)-2-oxo-1,2-dihydro-3H-indol-3-ylidene]-N,N-dimethylacetamide, (3E)-3-(2-azetidin-1-yl-2-oxoethylidene)-1-(cyclohexylmethyl)-1,3-dihydro-2H-indol-2-one, (3E)-3-(2-azetidin-1-yl-2-oxoethylidene)-1-(cyclopentylmethyl)-1,3-dihydro-2H-indol-2-one, (2E)-[1-(2-ethylbutyl)-2-oxo-1,2-dihydro-3H-indol-3-ylidene]acetic acid, (3E)-1-(2-ethylbutyl)-3-(2-oxo-2-pyrrolidin-1-ylethylidene)-1,3-dihydro-2-H-indol-2-one, (3E)-3-(2-azetidin-1-yl-2-oxoethylidene)-1-(2-ethylbutyl)-1,3-dihydro-2H-indol-2-one, (3E)-3-(2-azetidin-1-yl-2-oxoethylidene)-1-(cyclobutylmethyl)-1,3-dihydro-2H-indol-2-one, and (3E)-1-(cyclobutylmethyl)-3-(2-oxo-2-pyrrolidin-1-ylethylidene)-1,3-dihydro-2H-indol-2-one.

Embodiment 25: The method according to any one of embodiments 20-24, wherein said compound is a substantially pure enantiomer.

In certain embodiments any of the foregoing methods exclude the use of natural products other than podocarpic acid and/or a podocarpic acid derivative. In certain embodiments the foregoing methods additionally or alternatively exclude the use of one or more of the following vitamin C, benfotiamine, pyridoxamine, alpha-lipoic acid, taurine, pimagedine, aspirin, carnosine, metformin, pioglitazone, pentoxifylline, resveratrol, and curcumin.

### DEFINITIONS

Unless otherwise indicated, reference to a compound (e.g., to a TRPA1 activator (e.g., podocarpic acid or a derivative and/or analog thereof) as described herein) should be construed broadly to include pharmaceutically acceptable salts, prodrugs, tautomers, alternate solid forms, non-covalent complexes, and combinations thereof, of a chemical entity of the depicted structure or chemical name.

Generally, reference to a certain element such as hydrogen or H is meant to include all isotopes of that element. For example, if an R group is defined to include hydrogen or H, it also includes deuterium and tritium. Accordingly, isotopically labeled compounds are within the scope of this invention.

A pharmaceutically acceptable salt is any salt of the parent compound that is suitable for administration to an animal or human. A pharmaceutically acceptable salt also refers to any salt which may form in vivo as a result of administration of an acid, another salt, or a prodrug which is converted into an acid or salt. A salt comprises one or more ionic forms of the compound, such as a conjugate acid or base, associated with one or more corresponding counterions. Salts can form from or incorporate one or more deprotonated acidic groups (e.g. carboxylic acids), one or more protonated basic groups (e.g. amines), or both (e.g. zwitterions).

A prodrug is a compound that is converted to a therapeutically active compound after administration. For example, conversion may occur by hydrolysis of an ester group, such as a C₁-C₆ alkyl ester of the carboxylic acid group of the present compounds, or some other biologically labile group. Prodrug preparation is well known in the art. For example, “Prodrugs and Drug Delivery Systems,” which is a chapter in Richard B. Silverman, Organic Chemistry of Drug Design and Drug Action, 2d Ed., Elsevier Academic Press: Amsterdam, 2004, pp. 496-557, provides further detail on the subject.

Tautomers are isomers that are in equilibrium with one another. For example, tautomers may be related by transfer of a proton, hydrogen atom, or hydride ion.

Unless stereochemistry is explicitly depicted, a structure is intended to include every possible stereoisomer, both pure or in any possible mixture.

Alternate solid forms are different solid forms than those that may result from practicing the procedures described herein. For example, alternate solid forms may be polymorphs, different kinds of amorphous solid forms, glasses, and the like. In various embodiments alternate solid forms of any of the compounds described herein are contemplated.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group will be substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, alkynoxy, aryloxy, aralkyloxy, heterocyclyloxy, and heterocyclylalkoxy groups; carbonyls (oxo); carboxyls; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN), and the like.

The term “alkyl” refers to and covers any and all groups that are known as normal alkyl, branched-chain alkyl, cycloalkyl and also cycloalkyl-alkyl. Illustrative alkyl groups include, but are not limited to methyl, ethyl, n-propyl, isopropyl, n-butyl, isobutyl, sec-butyl, t-butyl, octyl, and decyl. The term “cycloalkyl” refers to cyclic, including polycyclic, saturated hydrocarbyl groups. Examples include, but are not limited to cyclopentyl, cyclohexyl, dicyclopentyl, norbornyl, octahydronapthyl, and spiro[3.4]octyl. In certain embodiments, alkyl groups contain 1-12 carbon atoms (C1-12 alkyl), or 1-9 carbon atoms (C₁₋₉ alkyl), or 1-6 carbon atoms(C₁₋₆ alkyl), or 1-5 carbon atoms (C₁₋₅ alkyl), or carbon atoms (C₁₋₄ alkyl), or 1-3 carbon atoms (C₁₋₃ alkyl), or 1-2 carbon atoms (C₁₋₂ alkyl).

By way of example, the term “C₁₋₆ alkyl group” refers to a straight chain or branched chain alkyl group having 1 to 6 carbon atoms, and may be exemplified by a methyl group, an ethyl group, an n-propyl group, an isopropyl group, an n-butyl group, an isobutyl group, a tert-butyl group, a sec-butyl group, an n-pentyl group, a tert-amyl group, a 3-methylbutyl group, a neopentyl group, and an n-hexyl group.

The term “alkoxy” as used herein means an alkyl group bound through a single, terminal oxygen atom. An “alkoxy” group may be represented as O-alkyl where alkyl is as defined above. The term “aryloxy” is used in a similar fashion, and may be represented as —O-aryl, with aryl as defined below. The term “hydroxy” refers to —OH.

Similarly, the term “alkylthio” as used herein means an alkyl group bound through a single, terminal sulfur atom. An “alkylthio” group may be represented as —S-alkyl where alkyl is as defined above. The term “arylthio” is used similarly, and may be represented as —S-aryl, with aryl as defined below. The term “mercapto” refers to —SH.

Aryl groups are cyclic aromatic hydrocarbons that do not contain heteroatoms. Aryl groups include monocyclic, bicyclic and polycyclic ring systems. Thus, aryl groups include, but are not limited to, phenyl, azulenyl, heptalenyl, biphenylenyl, indacenyl, fluorenyl, phenanthrenyl, triphenylenyl, pyrenyl, naphthacenyl, chrysenyl, biphenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

The term “heteroaryl group” refers to a monocyclic or condensed-ring aromatic heterocyclic group containing one or more hetero-atoms selected from O, S and N. If the aromatic heterocyclic group has a condensed ring, it can include a partially hydrogenated monocyclic group. Examples of such a heteroaryl group include a pyrazolyl group, a thiazolyl group, an isothiazolyl group, a thiadiazolyl group, an imidazolyl group, a furyl group, a thienyl group, an oxazolyl group, an isoxazolyl group, a pyrrolyl group, an imidazolyl group, a (1,2,3)- and (1,2,4)-triazolyl group, a tetrazolyl group, a pyranyl group, a pyridyl group, a pyrimidinyl group, a pyrazinyl group, a pyridazinyl group, a quinolyl group, an isoquinolyl group, a benzofuranyl group, an isobenzofuranyl group, an indolyl group, an isoindolyl group, an indazolyl group, a benzoimidazolyl group, a benzotriazolyl group, a benzoxazolyl group, a benzothiazolyl group, a benzo[b]thiophenyl group, a thieno[2,3-b]thiophenyl group, a (1,2)- and (1,3)-benzoxathiol group, a chromenyl group, a 2-oxochromenyl group, a benzothiadiazolyl group, a quinolizinyl group, a phthalazinyl group, a naphthyridinyl group, a quinoxalinyl group, a quinazolinyl group, a cinnolinyl group, and a carbazolyl group.

A “derivative” of a compound means a chemically modified compound wherein the chemical modification takes place at one or more functional groups of the compound. The derivative however, is expected to retain, or enhance, the pharmacological activity of the compound from which it is derived.

As used herein, “administering” refers to local and systemic administration, e.g., including enteral, parenteral, pulmonary, and topical/transdermal administration. Routes of administration for agents (e.g., TRPA1 activator(s) described herein, or a tautomer(s) or stereoisomer(s) thereof, or pharmaceutically acceptable salts or solvates of said activator(s), said stereoisomer(s), or said tautomer(s), or analogues, derivatives, or prodrugs thereof) that find use in the methods described herein include, e.g., oral (per os (p.o.)) administration, nasal or inhalation administration, administration as a suppository, topical contact, transdermal delivery (e.g., via a transdermal patch), intrathecal (IT) administration, intravenous (“iv”) administration, intraperitoneal (“ip”) administration, intramuscular (“im”) administration, intralesional administration, or subcutaneous (“sc”) administration, or the implantation of a slow-release device e.g., a mini-osmotic pump, a depot formulation, etc., to a subject. Administration can be by any route including parenteral and transmucosal (e.g., oral, nasal, vaginal, rectal, or transdermal). Parenteral administration includes, e.g., intravenous, intramuscular, intra-arterial, intradermal, subcutaneous, intraperitoneal, intraventricular, ionophoretic and intracranial. Other modes of delivery include, but are not limited to, the use of liposomal formulations, intravenous infusion, transdermal patches, etc.

The terms “systemic administration” and “systemically administered” refer to a method of administering the agent(s) described herein or composition to a mammal so that the agent(s) or composition is delivered to sites in the body, including the targeted site of pharmaceutical action, via the circulatory system. Systemic administration includes, but is not limited to, oral, intranasal, rectal and parenteral (e.g., other than through the alimentary tract, such as intramuscular, intravenous, intra-arterial, transdermal and sub cutaneous) administration.

The term “effective amount” or “pharmaceutically effective amount” refers to the amount and/or dosage, and/or dosage regime of one or more agent(s) necessary to bring about the desired result e.g., an amount sufficient to to ameliorate one or more symptoms of the pathology (e.g., a pathology characterized by advanced glycation endproducts such as diabetes or a complication thereof), and/or to slow or stop the onset of the pathology, and/or to lower the level of α-dicarbonyl compounds, and so forth.

As used herein, the terms “treating” and “treatment” refer to delaying the onset of, retarding or reversing the progress of, reducing the severity of, or alleviating or preventing either the disease or condition to which the term applies, or one or more symptoms of such disease or condition.

The term “mitigating” refers to reduction or elimination of one or more symptoms of a pathology or disease, and/or a reduction in the rate or delay of onset or severity of one or more symptoms of that pathology or disease, and/or the prevention of that pathology or disease. In certain embodiments, the reduction or elimination of one or more symptoms of pathology or disease can include, but is not limited to, reduction or elimination of one or more markers that are characteristic of the pathology or disease (e.g., AGE levels).

As used herein, the phrase “consisting essentially of” refers to the genera or species of active pharmaceutical agents recited in a method or composition, and further can include other agents that, on their own do not substantial activity for the recited indication or purpose.

The terms “subject”, “individual”, and “patient” interchangeably refer to a mammal, preferably a human or a non-human primate, but also domesticated mammals (e.g., canine or feline), laboratory mammals (e.g., mouse, rat, rabbit, hamster, guinea pig) and agricultural mammals (e.g., equine, bovine, porcine, ovine). In various embodiments, the subject can be a human (e.g., adult male, adult female, adolescent male, adolescent female, male child, female child) under the care of a physician or other health worker in a hospital, psychiatric care facility, as an outpatient, or other clinical context. In certain embodiments the subject may not be under the care or prescription of a physician or other health worker.

The term “formulation” or “drug formulation” or “dosage form” or “pharmaceutical formulation” as used herein refers to a composition containing at least one therapeutic agent or medication for delivery to a subject. In certain embodiments the dosage form comprises a given “formulation” or “drug formulation” and may be administered to a patient in the form of a lozenge, pill, tablet, capsule, suppository, membrane, strip, liquid, patch, film, gel, spray or other form.

The term “substantially pure ” means sufficiently homogeneous to appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical or chemical properties, of the compound. Methods for purification of the compounds to produce substantially chemically pure compounds are known to those of skill in the art. A substantially chemically pure compound may, however, be a mixture of stereoisomers or isomers. In such instances, further purification might increase the specific activity of the compound.

The term “substantially pure” when used with respect to enantiomers indicates that one particular enantiomer (e.g. an S enantiomer or an R enantiomer) is substantially free of its stereoisomer. In various embodiments substantially pure indicates that a particular enantiomer is at least 70%, or at least 80%, or at least 90%, or at least 95%, or at least 98%, or at least 99% of the purified compound. Methods of producing substantially pure enantiomers are well known to those of skill in the art. For example, a single stereoisomer, e.g., an enantiomer, substantially free of its stereoisomer may be obtained by resolution of the racemic mixture using a method such as formation of diastereomers using optically active resolving agents (Stereochemistry of Carbon Compounds, (1962) by E. L. Eliel, McGraw Hill; Lochmuller (1975) J. Chromatogr., 113(3): 283-302). Racemic mixtures of chiral compounds of the can be separated and isolated by any suitable method, including, but not limited to: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. Another approach for separation of the enantiomers is to use a Diacel chiral column and elution using an organic mobile phase such as done by Chiral Technologies (www.chiraltech.com) on a fee for service basis.

### BRIEF DESCRIPTION OF THE DRAWINGS

FIG. 1 illustrates the structure of podocarpic acid.

FIG. 2, panels A-J: Establishing C. elegans glod-4 as a viable model for studying a-DC-related pathologies. Structures of endogenous reactive a-DCs. In vivo a-DC detoxification is primarily mediated via glutathione (GSH)-dependent glyoxalase I/II (human GLO½) and the co-factor-independent glyoxalase, DJ1 (human). GLOD-4 and DJR-1.1, DJR-1.2 are the C. elegans orthologs, of the mammalian GLO1 and DJ1, respectively. (A) Levels of MGO (left) and GO (right) in wild-type (N2) and glod-4. (B) Age-dependent change in sensitivity to touch, quantified by the touch index (TI) in N2 and glod- 4. (C) Age-dependent change in number of body bends (swim bends) in liquid media for N2 and glod-4. n=30. (D) Neuronal damage in unc-33p::gfp (pan-neuronal GFP) at days 1, 4, 7, and 10 of adulthood reared on empty vector (EV, L4440) or glod-4 RNAi. n=45. (E) Survival curves for N2 and glod-4 animals reared on OP50-1. (F) Levels of MGO in glod-4, reared on media supplemented with 0 or 2% glucose. (G) Neuronal damage in glod-4;unc-33p::gfp (pan-neuronal GFP) on day 4 of adulthood, reared on media supplemented with 0 or 2% glucose. n=45. (H) Survival curves for glod-4 animals reared on media supplemented with 0 or 2% glucose. (I) Progression of age-dependent phenotypes observed in glod-4 mutants, which form the basis of using this mutant as a model to study diabetes-related pathologies. All error bars represent SD. See also FIGS. 9 and 10.

FIG. 3, panels A-G: SKN-1/Nrf2 renders physiological protection against a-DC-induced toxicity in C. elegans. (A) Quantification of GFP foci in gst-4p::gfp) and glod-4;gst-4p::gfp animals reared on empty vector (EV, L4440) or skn-1 RNAi. n=15. (B) qPCR analysis of SKN-1 target genes, gcs-1 and gst-4 in N2 and glod-4 reared on EV or skn-1 RNAi. The data is normalized to the corresponding expression levels in N2 reared on EV (represented by the dotted line). (C) Survival curves for N2 and skn-1 mutant animals reared on EV or glod-4 RNAi. (D) Touch indices during young adult stage for N2, skn-1, and animals with transgenic expression of skn-1 only in ASI neurons (skn-1b) or only in the intestine (skn-1c). gpa-4 and ges-1 promoters were used to drive skn-1 expression in the ASI neuron and intestine, respectively. (E) Survival curves for N2, skn-1, and animals with transgenic expression of skn-1 only in ASI neurons (skn-1b) or only in the intestine (skn-1c) under glod-4 RNAi. (F) Quantification of GFP foci in glod-4;gst-4p::gfp animals reared on EV, pmk-1, sek-1, or sgk-1 RNAi. n=15. (G) Survival curves for N2, pmk-1, sgk-1, and sek-1 mutant animals under glod-4 RNAi. All error bars represent SD. See also FIG. 11.

FIG. 4, panels A-I: TRPA-1 is a sensor for a-DCs and result in SKN-1/Nrf2 activation. (A) Survival curves for RNAi knockdown of N2 and various C. elegans TRP channel mutants under glod-4 RNAi. (B) Survival curves for N2 and animals with transgenic expression of trpa-1 only in the intestine (XuEx601), neurons (XuEx606), muscle (XuEx610), or hypodermis (XuEx611), reared on glod-4 RNAi. ges-1, rgef-1, myo-3, and dpy-7 promoters were used to drive trpa-1 expression in the intestine, neurons, muscle, and hypodermis, respectively. (C) Touch indices during young adult stage for glod-4 animals reared on empty vector (EV, L4440) or trpa-1 RNAi. (D) Neuronal damage in glod-4;unc-33p::gfp (pan-neuronal GFP) on day 5 of adulthood, reared on EV, trpa-1, or skn-1 RNAi. (E) Quantification of GFP foci in gst-4p::gfp and glod-4;gst-4p::gfp animals reared on EV or trpa-1 RNAi. n=15. (F) qPCR analysis of SKN-1 target genes, gcs-1 and gst-4 in glod-4 mutants reared on EV or trpa-1 RNAi. The data is normalized to the corresponding expression levels in glod-4 mutants reared on EV (represented by the dotted line). (G) Percentage change in peak fluorescence intensity observed in transgenic animals expressing intestinal GCaMP1.3 (Ca²⁺ sensor) and containing a wild-type TRPA-1 or TRPA-1^{E}1018A (CA²⁺ impermeable) mutant channel in response to MGO or water (control). n=15. (H) Quantification of GFP foci in glod-4;gst-4p::gfp animals reared on EV, or several Ca⁺²-sensitive kinase RNAis: unc-43, cmk-1, and pkc-2. n=15. (I) Survival curves for N2, unc-43, cmk-1, and pkc-2 animals under glod-4 RNAi condition. All error bars represent SD. See also FIG. 12.

FIG. 5, panels A-F: Downstream glyoxalases mediate a-DC detoxification in response to TRPA-1/SKN-1 activation. (A) Quantification of GFP intensity of glod-4p::gfp reporter strain, reared on empty vector (EV, L4440), skn-1, or trpa-1 RNAi, supplemented with water (control) or MGO (7 mM). n=10. (B) Levels of MGO in N2 and glod-4 animals, reared on EV, trpa-1, or skn-1 RNAi. (C) qPCR analysis of conserved glyoxalases, glod-4, djr-1.1, and djr-1.2, in N2 and glod-4, reared on EV, skn-1, or trpa-1 RNAi. The data is normalized to the corresponding expression levels in N2 animals reared on EV (represented by the dotted line). (D) Levels of MGO in N2, reared on EV, glod-4, djr-1.1, or djr-1.2 RNAi. (E) Touch indices during young adult stage for N2, reared on EV, glod-4, djr-1.1, and djr-1.2 RNAi. (F) Survival curves for N2, reared on EV, glod-4, djr-1.1, and djr-1.2 RNAi. All error bars represent SD. See also FIG. 13.

FIG. 6, panels A-I: podocarpic acid (PA) is a TRPA-1 agonist and SKN-1/Nrf2 activator that ameliorates pathogenic phenotypes of C. elegans glod-4. (A) Structures of podocarpic acid (PA), a-lipoic acid (LA), and uridine monophosphate (UMP). (B) Touch indices in glod-4, supplemented with EtOH (control) or PA (20 pM) during young adult stage or day 8 of adulthood. (C) Neuronal damage in pan-neuronal GFP animals (unc-33p::gfp) at day 10 of adulthood, reared on empty vector (EV, L4440) or glod-4 RNAi, supplemented with EtOH (control) or PA (20 pM). n=45. (D) Survival curves for glod-4 mutant animals supplemented with EtOH (control), PA, LA, or UMP (20 pM). (E) Quantification of GFP foci in gst-4p::gfp animals reared on EV or glod-4 RNAi, supplemented with EtOH (control), PA, LA, or UMP (20 pM). n=10. (F) Quantification of GFP foci in glod-4;gst-4p::gfp animals reared on EV or trpa-1 RNAi, supplemented with EtOH (control), PA or LA (20 pM). n=15. (G) Percentage change in peak fluorescence intensity observed in transgenic animals expressing intestinal GCaMP1.3 (Ca²⁺ sensor) and a wild-type TRPA-1 or TRPA-1^{E1018A} (Ca²⁺ impermeable) mutant channel, in response to EtOH (control), PA or LA (20 pM). n=10. (H) Levels of MGO in glod-4, supplemented with EtOH (control), PA, LA, or UMP (20 pM) or in (I) glod-4, reared on EV or trpa-1 RNAi, supplemented with EtOH (control) or PA. All error bars represent SD. See also FIG. 14.

FIG. 7, panels A-F: Methylglyoxal (MGO)-induced neurotoxicity is sensed and rescued through a conserved mechanism. Fluorescence ratio (555/484 nm) changes for the membrane-permeable Ca⁺² indicator Rhod-3 AM (left) and representative Rhod-3 AM images (Scale bar is 10 pm) in pseudo color scale (right) for HEK293T cells transfected with: (A) TRPA1 (RAT) and GFP or GFP only. Cells were treated with 100 pM or 1 mM MGO and images captured before (-MGO) or 100 s after 100 pM MGO application. n≧6. (B) TRPA1 (worm) and GFP or GFP only, treated and imaged as in FIG. 7, panel A. n≧5. (C) TRPM8 (mouse) and GFP or GFP only. Cells were treated with 100 pM MGO first and then switched to 100 pM menthol. Images were captured after 100 s of incubation with MGO and menthol. n=9. (D) DIC images for differentiated 50B11 cells (immortalized rat DRG neuronal cells) treated with water (control), podocarpic acid/PA (250 pM), MGO (250 pM), or a combination of PA and MGO (each at 250 pM). Shrinkage in cell bodies (red dotted circles), retraction in neurite outgrowth (red arrows) and diminished neuronal networking is visible in MGO (only)-treated cells. Amelioration of size of the cell bodies (white dotted circle) and length of neurite outgrowth emerging from the edge of the soma (white arrows) due to PA treatment. Scale bar 50 pm. (D) Neurite length and (F) soma size quantification in rat DRG neuronal cells, treated with water (control), 250 pM PA, 250 pM MGO, and 250 pM each of MGO and PA. All error bars represent SD. See also FIG. 15.

FIG. 8: Model for a-DC (endogenous stress)- or cold (exogenous stress)-induced TRPA-1 activation and subsequent divergence of downstream signaling. TRPA-1/TRPA1 activation via a-DCs is relayed through UNC-43 (C. elegans CaMKII), PMK-1, and SEK-1 (C. elegans MAPK) to SKN-1/Nrf2 resulting in the expression of various downstream glyoxalases to achieve organism-wide a-DC detoxification. In contrast, the effects of cold-induced TRPA-1 activation is mediated through DAF-16/FOXO regulation via PKC-2 (C. elegans protein kinase C) and SGK-1 (C. elegans serum- and glucocorticoid-inducible kinase). Drug-induced activation of TRPA1-Nrf2 ameliorates pathologies associated with elevated a-DC buildup.

FIG. 9, panels A-D, LC-MS/MS-based estimation of a-DCs and Neuronal damage due to glod-4 knockdown. Related to FIG. 2. (A) Reaction conditions for derivatization of a-DCs with o-phenylenediamine (OPD). (B) Extracted ion chromatograms (EICs) for MRM transitions, characteristic of OPD derivatives for synthetic 3DG, GO, and MGO (each compound injected at 5 pmol). 2,3-Hexanedione is used as internal standard (IS). (C) Levels of 3DG in N2 and glod-4 animals. (D) Fluorescence microscopy images of unc-33p::gfp (pan-neuronal GFP) reared on empty vector (EV, L4440) or glod-4 RNAi at days 1, 4, 7, and 10 of adulthood. Arrows and asterisk mark areas for comparison between EV and glod-4 RNAi fed animals. Damages in the neuronal processes are classified as thinning or break in dendrites, neuronal waviness, or reduced fluorescence in the nerve ring. Scale bar 20 pm.

FIG. 10, panels A-F, Pathogenic phenotypes due to MGO treatment on N2 and glucose on glod-4. Related to FIG. 2. (A) Touch index (TI) during young adult stage for N2 animals, supplemented with water (control) or 7 mM MGO. (B) Number of body bends in liquid media for N2 animals supplemented with water (control) or 7 mM MGO. (C) Fluorescence microscopy images of unc-33p::gfp (pan-neuronal GFP) supplemented with water (control) or 7 mM MGO. Arrows and asterisk mark areas for comparison between control and 7 mM MGO-treated animals. Damages in the neuronal processes are classified as thinning or break in dendrites, neuronal waviness or reduced fluorescence in the nerve ring. Scale bar 20 pm. (D) Quantification of the extent of neuronal damage in pan-neuronal GFP animals (unc-33p::gfp) at day 4 of adulthood reared supplemented with water (control) or 7 mM MGO. n=45. (E) Survival curves for N2 animals supplemented with water (control) or 7 mM MGO. (F) Fluorescence microscopy images of glod-4;otls117[unc-33p::gfp] (pan-neuronal GFP) reared on media supplemented with 0 or 2% glucose at day 4 of adulthood. Arrows point to damages in the neuronal processes in 2% glucose-treated animals, either as thinning/break in dendrites, or neuronal waviness. Red asterisk marks reduced fluorescence in the nerve ring of glucose-treated animals compared to control. Scale bar 20 pm.

FIG. 11, panels A-E, SKN-1/Nrf2 activation due to exogenous MGO treatment and specificity of downstream response to a-DC stress. Related to FIG. 3. (A) Quantification (left) and fluorescence microscopy images (right) of GFP foci in gst-4p::gfp animals reared on empty vector (EV, L4440) or skn-1 RNAi supplemented with water (control) or 7 mM MGO. Foci are marked by white arrows. n=91. Scale bar 0.15 mm. (B) Relative quantification of animals exhibiting different fluorescence levels (left) and microscopy images (right) of GFP intensities in gcs-1p::gfp animals supplemented with water (control) or 7 mM MGO. Animals were categorized as follows: ‘high’ for strong GFP signal throughout the intestine, ‘medium’ for GFP signal in the anterior or posterior section of the intestine and ‘low’ for weak or no signal. n=100. (C) qPCR analysis of SKN-1 target genes, gst-4 and gcs-1 in N2 animals reared on EV or skn-1 RNAi supplemented with water (control, −) or 7 mM MGO (+). The data is normalized to the corresponding expression levels in N2 (EV, water), represented by the dotted line. (D) Fluorescence microscopy images depicting nuclear localization of DAF-16::GFP and SKN-1::GFP supplemented with water (control) or 7 mM MGO. Heat shock was used as a positive control to drive DAF-16 nuclear localization. Red arrows point towards nuclear localized transcription factors (SKN-1 and DAF-16). Scale bar 20 pm. (E) Fluorescence microscopy images to observe GFP foci in glod-4;gst-4p::gfp animals reared on empty vector (EV, L4440), sgk-1, sek-1, or pmk-1 RNAi. Scale bar 20 pm.

FIG. 12, panels A-F, TRPA-1 is a sensor for a-DCs and result in SKN-1/Nrf2 activation. Related to FIG. 4. (A) Survival curves for N2 and trpa-1 mutant animals reared on empty vector (EV, L4440) or glod-4 RNAi. (B) Fluorescence microscopy images of glod-4;unc-33p::gfp (pan-neuronal GFP) reared on EV, trpa-1, or skn-1 RNAi during day 5 of adulthood. Red arrows point to damages due to trpa-1 or skn-1 knockdown in the neuronal processes: discontinuity in dendrites, breaks in commissures, thinning of neuronal processes, along the length of the body from head to tail at multiple areas. Scale bar 20 pm. (C) Quantification (top) and fluorescence microscopy images (bottom) of GFP foci in gst-4p::gfp animals reared on EV or trpa-1 RNAi supplemented with water (control) or 7 mM MGO. n=91. Scale bar 0.15 mm. (D) qPCR analysis of SKN-1 target genes, gst-4 and gcs-1 in N2 animals reared on EV or skn-1 RNAi supplemented with water (control, -) or 7 mM MGO (+).The data is normalized to the corresponding expression levels in N2 (EV, water) represented by the dotted line. (E) Fluorescence intensity traces for transgenic animals expressing an intestinal GCaMP1.3 (Ca²⁺ sensor) containing a wild-type TRPA-1 (WT) or TRPA-1^{E1018A} (Ca²⁺ impermeable) channel mutant in response to 7 mM MGO or water. Each colored line indicates the trace for a single animal. (F) Fluorescence microscopy images to observe GFP foci in glod-4;gst-4p::gfp animals reared on EV, pkc-2, unc-43, or cmk-1 RNAi. Scale bar 20 pm.

FIG. 13, panels A-C, downstream glyoxalases mediate a-DC detoxification in response to TRPA-1/SKN-1 activation. Related to FIG. 5. (A) Fluorescence microscopy representing GLOD-4 expression in glod-4p::gfp animals reared on empty vector (EV, L4440), skn-1, or trpa-1 RNAi, supplemented with water (control) or 7 mM MGO. Scale bar 20 pm. (B) Levels of MGO in N2 and glod-4 animals. Animals were reared on EV, trpa-1, or skn-1 RNAi. (C) Levels of GO in N2 reared on EV, glod-4, djr-1.1, or djr-1.2 RNAi.

FIG. 14, panels A-D, podocarpic acid (PA) and a-Lioic acid (LA) rescues glod-4 phenotypes and TRPA1 activation by allyl isothiocyanate (AITC) or MGO. Related to FIG. 6. (A) Touch indices (performed as part of a drug screen of TimTec NPL640) during young adult stage for glod-4, supplemented with DMSO (control), PA, or UMP (66.7 pM in DMSO). Scale bar 10 pm. (B) Fluorescence microscopy images of unc-33p::gfp (pan-neuronal GFP) animals at day 10 supplemented with EtOH (control) or PA (20 pM), reared on empty vector (EV, L4440) or glod-4 RNAi. Asterisks indicate regions of interest that have been magnified. Arrows point to damages in the neuronal processes in animals reared on glod-4 RNAi treated with EtOH (no drug), either as dendritic breaks, neuronal waviness or reduced fluorescence in the nerve ring. (C) Fluorescence intensity traces in transgenic animals expressing an intestinal Ca sensor GCaMP1.3 containing a wild-type TRPA-1 channel (WT) or a Ca impermeable channel mutant (TRPA-1^{E1018A}) in response to ethanol (control), or 20 pM of PA or LA. Each colored line indicates the trace for a single animal. (D) Levels of GO in N2 and glod-4 animals treated with EtOH (control), PA, LA, or UMP (20 pM each).

FIG. 15, panels A-B, podocarpic acid (PA) and a-Lioic acid (LA) rescues glod-4 phenotypes and TRPA1 activation by allyl isothiocyanate (AITC) or MGO. Related to FIG. 7. Fluorescence ratio (555/484 nm) changes for the membrane-permeable Ca⁺² indicator Rhod-3 AM in HEK293 cells transfected with: (A) TRPA1 (rat) and GFP or GFP only. Cells were treated with 100 pM allyl isothiocyanate (AITC). (B) TRPA1 (worm) and GFP or GFP only. Cells were treated with 100 pM AITC first and then switched to 100 pM MGO.

### DETAILED DESCRIPTION

The pathogenesis of various diabetes-related complications is best explained by an age-dependent accumulation of glucose-derived reactive byproducts α-dicarbonyl compounds (α-DCs), e.g., methylglyoxal (MGO). However, such pathologies take several years to develop in humans making it quite challenging to study the underlying biochemical pathways regulating α-DC stress and associated toxicity. Consequently, the conventional treatment regimen for long-term diabetics is focused primarily on lowering their blood glucose levels. In various embodiments an orthogonal treatment approach is provided that involves bolstering the organismal capability to detoxify reactive α-DCs.

The findings described herein are facilitated, inter alia, by the development of a Caenorhabditis elegans-based model to study α-DC stress-related pathologies relevant to diabetes, such as hyperesthesia, nerve damage, and early mortality in a two-week span. We have undertaken a multidisciplinary approach, utilizing the worm model's ease of genetic manipulation to identify TRPA1 as a conserved sensor for α-DC stress that activates an Nrf2-dependent α-DC detoxification network. In this work, we identify some of the key aspects of this regulatory pathway: 1) TRPA-1/TRPA1 acts as a sensor for α-DC stress resulting in an influx of Ca⁺² ions; 2) transduction of the ensuing signal to SKN-1/Nrf2 via UNC-43 (Ca⁺²/Calmodulin Kinase II), PMK-1 and SEK-1 (p38 MAP kinases); and 3) SKN-1-dependent transcriptional regulation of glutathione-dependent (GLO1) and -independent (DJ1) glyoxalases. We also observe that several key aspects of this pathway are conserved in mammalian cells. Interestingly, this pathway is in stark contrast to the TRPA-1/Ca⁺² flux implicated in cold sensation in C. elegans , where PKC-2, SGK-1 (kinases), and DAF-16/FOXO (transcription factor) are involved (Xiao et al., Cell). The results thus suggest a TRPA-1 signaling plasticity in deciding the organism's response to an endogenous (α-DC) vs. an exogenous (cold) stress.

The identification of TRPA1-Nrf2 signaling has tremendous therapeutic potential. There are many known TRPA1 activators (active components of mustard, wasabi, cinnamon, etc.), but until now there has been no indication that these may be used for treating diabetic pathologies (or other pathologies associated with AGEs). Using the model system a phenotypic drug screen was performed to identify other TRPA1 activators.

Thus, a phenotypic drug screen in our C. elegans model using a natural product library, identified podocarpic acid as a novel TRPA1 activator. Podocarpic acid not only ameliorates the pathogenic phenotypes (due to α-DC stress) in C. elegans , but also in mammalian dorsal root ganglion (DRG) neuronal cells. Subsequently, we also identify TRPA1-Nrf2 activation by α-lipoic acid, a drug prescribed for diabetic neuropathy in humans, as a novel mode of action for this drug. Our results underscore the importance of using C. elegans models, not only to understand the underlying biochemistry of a disease, but also for high-throughput drug screening and accelerated lead identification.

The methods and agents described herein facilitate the creation of a rapid and deliverable pipeline (currently non-existent) for developing drugs to treat diabetic complications and/or other pathologies characterized by the formation and/or accumulation of AGEs. The results described herein indicate that amelioration of α-DC stress is a viable therapeutic option for treating diabetic complications. Since α-DC stress has also been associated with neurodegenerative disorders, for which diabetes is an additional risk factor, such as Alzheimer's disease, Parkinson's disease, and ATTR amyloidosis, the work described herein is of broader clinical relevance.

Moreover the formation and accumulation of advanced glycation endproducts (AGEs) has been implicated in the progression of age-related diseases (Tan et al. (2006) Sleep, 29(3): 329-333). AGEs have been implicated in Alzheimer's Disease (Srikanth and Maczurek (2009) Neurobiol. Aging, 32(5): 763-777), cardiovascular disease (Simm et al. (2007) Esp. Gerontol., 42(7): 668-675), and stroke (Zimmerman et al. (1995) Proc. Natl. Acad. Sci. USA, 92(9):3744-3748). One mechanism by which AGEs induce damage is through a process called cross-linking that causes intracellular damage and apoptosis (Shaikh and Nicholson (2008) J. Neurosci. Res. 86(9):2071-2082). They form photosensitizers in the crystalline lens (Fuentealba (2009) Photochem. Photobiol. 85(1): 185-194), which has implications for cataract development (Gul et al. (2009) Graefes. Arch. Clin. Exp. Ophthalmol. 247(6): 809-814). Reduced muscle function is also associated with AGEs (Haus et al. (2007) J. Appl. Physiol., 103(6): 2068-2076).

AGEs have a range of pathological effects, such as: 1) Increased vascular permeability; 2) Increased arterial stiffness; 3) Inhibition of vascular dilation by interfering with nitric oxide; 4) Oxidizing LDL; 5) Binding cells including macrophages, endothelial cells, and mesangial cells to induce the secretion of a variety of cytokines; and 6) Enhanced oxidative stress (see, e.g., Gugliucci and Bendayan (1996) Diabetologia 39(2): 149-160; Yan et al. (2007) Chin. Med. J. 120(9): 787-793; and the like. In view of results described herein it is believed that TRPA1 activators find utility, inter alia, in the treatment and prophylaxis of these conditions.

Accordingly, in certain embodiments, a method for the treatment or prophylaxis of diabetes in a mammal, is provided where the method comprises administering to a mammal identified as having diabetes or pre-diabetes an agent that activates TRPA1 in an amount sufficient to ameliorate one or more symptoms of diabetes or pre-diabetes. In certain embodiments the amount sufficient to ameliorate one or more symptoms of diabetes or pre-diabetes is an amount sufficient to ameliorate a complication of diabetes selected from the group consisting of diabetic neuropathy (e.g., peripheral neuropathy, a neurodegenerative condition, etc.), cardiomyopathy, nephropathy, retinopathy, microvascular damage, and early mortality.

In certain embodiments a method for ameliorating a pathology characterized by elevated α-dicarbonyl compounds (and/or advanced glycation endproducts) or prophylactically slowing or stopping the onset of such a pathology in a mammal, is provided where the method comprises administering to the mammal an agent that activates TRPA1 in an amount sufficient to activate TRPA1 and/or to ameliorate one or more symptoms of the pathology, and/or to slow or stop the onset of said pathology, and/or to lower the level of α-dicarbonyl compounds in the mammal. In certain embodiments the pathology is selected from the group consisting of Diabetes, Alzheimer's disease, Parkinson's disease, cataract formation, stroke, and cardiovascular disease.

In certain embodiments a method for reducing the levels of α-dicarbonyl and/or advanced glycation endproducts compounds in a mammal is provided where the method comprises administering to the mammal an agent that activates TRPA1 in an amount sufficient to lower the level of α-dicarbonyl compounds and/or advanced glycation endproducts in said mammal.

In certain embodiments a method for reducing the amount of or slowing or stopping the accumulation of advanced glycation endproducts in a mammal is provided where the method comprises administering to the mammal an agent that activates TRPA1 in an amount sufficient to slow or stop the accumulation of advanced glycation endproducts in said mammal.

### Active Agents.

As explained above, it was discovered that activators TRPA1 rescue α-DC-induced pathologies in C. elegans and mammalian cells. In view of the findings described herein it is believed that amelioration of α-DC stress represents a viable option to address related pathologies in diabetes and associated neurodegenerative conditions like Alzheimer's, and Parkinson's disease.

Moreover it was discovered that podocarpic acid was effective TRPA12 activator that appears to rescue α-DC-induced pathologies. Accordingly, in certain embodiments, podocarpic acid is utilized as an active agent in the methods described herein.

In view of the positive results obtained using podocarpic acid, it is believed that various podocarpic analogs and/or derivatives are also useful in the methods described herein.

Numerous podocarpic acid analogs and/or derivatives are well known to those of skill in the art (see, e.g., Cui et al. (2008) Bioorganic & Med. Chem. Lett. 18: 5197-5200; Nguyen (2004) Synthesis of a novel family of amide derivatives of podocarpic acid, M.S. Thesis, University of Central Florida, Orlando, Fla.; McKee et al. (2014) Austin J. Bioorg. & Org. Chem., 1(1): 1-7; and the like).

Accordingly, in certain embodiments, the methods described herein utilize one or more of the podocarpic acid derivatives shown in Table 1. Methods of making these podocarpic acid derivatives are described by Clui et al. (2008) Bioorganic & Med. Chem. Lett. 18: 5197-5200.

TABLE 1
Illustrative podocarpic acid derivatives from Clui et al. (2008)
Bioorganic & Med. Chem. Lett. 18: 5197-5200.

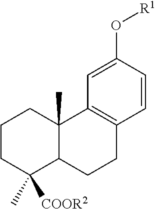

| Compound | R¹ | R² |
|---|---|---|
| 6a | H | H |
| 6b | Me | H |
| 6c | 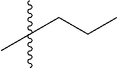 [SMILES: *CCC] | H |
| 6d | 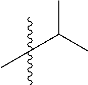 [SMILES: *C(C)C] | H |
| 6e | 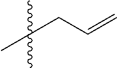 [SMILES: *CC=C] | H |
| 6f | 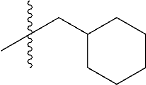 [SMILES: *CC1CCCCC1] | H |
| 6g | 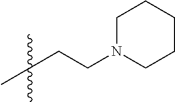 [SMILES: *CCN1CCCCC1] | H |
| 6h | 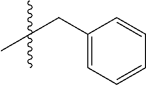 [SMILES: *Cc1ccccc1] | H |
| 6i | 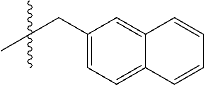 [SMILES: *Cc1ccc2ccccc2c1] | H |
| 6j | 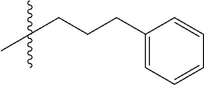 [SMILES: *CCCc1ccccc1] | H |
| 6k | 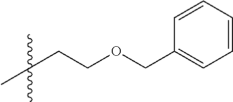 [SMILES: *CCOCc1ccccc1] | H |
| 6l | 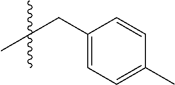 [SMILES: *Cc1ccc(C)cc1] | H |
| 6m | 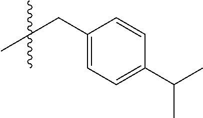 [SMILES: *Cc1ccc(C(C)C)cc1] | H |
| 6n | 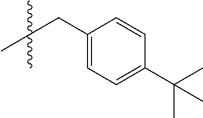 [SMILES: *Cc1ccc(C(C)(C)C)cc1] | H |
| 6o | 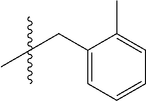 [SMILES: *Cc1ccccc1C] | H |
| 6p | 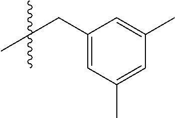 [SMILES: *Cc1cc(C)cc(C)c1] | H |
| 6q | 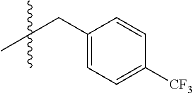 [SMILES: *Cc1ccc(C(F)(F)F)cc1] | H |
| 6r | 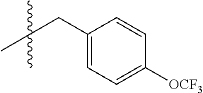 [SMILES: *Cc1ccc(OC(F)(F)F)cc1] | H |
| 6s | 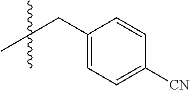 [SMILES: *Cc1ccc(C#N)cc1] | H |
| 6t | 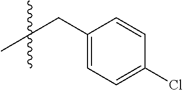 [SMILES: *Cc1ccc(Cl)cc1] | H |
| 6u | 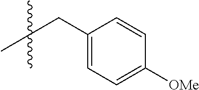 [SMILES: *Cc1ccc(OC)cc1] | H |
| 7a | H | CH₃ |
| 7j | 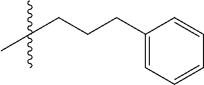 [SMILES: *CCCc1ccccc1] | CH₃ |

In certain embodiments, the methods described herein utilize one or more of the podocarpic acid derivatives shown in Table 2. Methods of making these podocarpic acid derivatives are described by Nguyen (2004) Synthesis of a novel family of amide derivatives of podocarpic acid, M.S. Thesis, University of Central Florida, Orlando, Fla.

TABLE 2
Illustrative amide derivatives of podocarpic acid (see, Nguyen (2004)
Synthesis of a novel family of amide derivatives of podocarpic acid,
M.S. Thesis, University of Central Florida, Orlando, Fl).

| Compound | Structure |
|---|---|
| Podocarpinol | 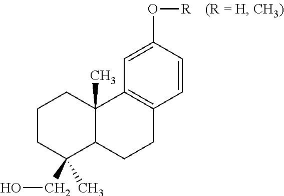 |
| Nimbiol | 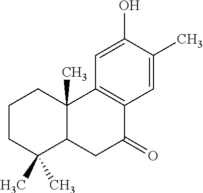 |
| N16 | 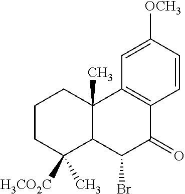 [SMILES: COC(=O)[C@@]1(C)CCC[C@]2(C)c3cc(OC)ccc3C(=O)[C@H](Br)C12] |
| N17 | 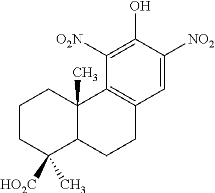 [SMILES: C[C@]1(C(=O)O)CCC[C@]2(C)c3c(cc([N+](=O)[O-])c(O)c3[N+](=O)[O-])CCC12] |
| N18 | 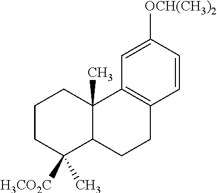 |
| N19 | 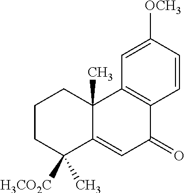 [SMILES: COC(=O)[C@@]1(C)CCC[C@@]2(C)C1=CC(=O)c1ccc(OC)cc12] |
| N20 | 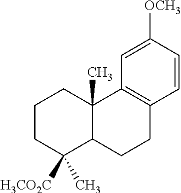 [SMILES: COC(=O)[C@@]1(C)CCC[C@]2(C)c3cc(OC)ccc3CCC12] |
| N22 | 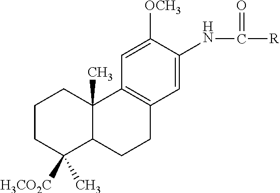 |
| N23 methyl-O- methylpodocarpate | 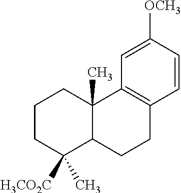 [SMILES: COC(=O)[C@@]1(C)CCC[C@]2(C)c3cc(OC)ccc3CCC12] |

As is evident, methods of making podocarpic acid analogs and/or derivatives are well known to those of skill. Illustrative methods include, but are not limited to 1) Substitution of electron-withdrawing groups onto C (13) of the aromatic C ring; 2) Introduction of different halogens at C (6) (Scheme 2); 3) Formation of the lactones from each 6 α-bromo methyl ester derivatives; and 4) Substitution of the methyl ester group at C (16) for an acetoxymethyl group as described by McKee et al. (2014) Austin J. Bioorg. & Org. Chem., 1(1): 1-7). Illustrative, but non-limiting list of compounds made using these methods is shown in Table 3. In certain embodiments, the methods described herein utilize one or more of the podocarpic acid derivatives shown in Table 3.

TABLE 3
Illustrative podocarpic amide derivatives (see, e.g., McKee et al. (2014)
Austin J. Bioorg. & Org. Chem., 1(1): 1-7).

| Compound(s) | Structure |
|---|---|
| MK10 | 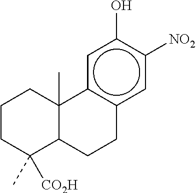 |
| MK11 | 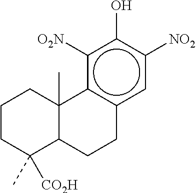 |
| MK12 (R = H) MK13 (R = NO₃) MK12a (R = Cl) MK12b (R = F) MK12c (R = I) | 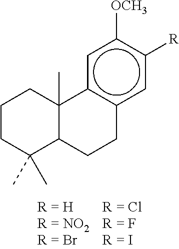 |
| MK12 MK13 MK13a MK13b MK13c MK14 | 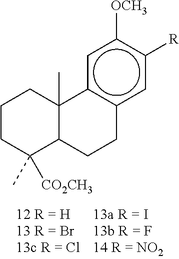 |
| MK15 MK16 MK16a MK16b MK16c MK17 | 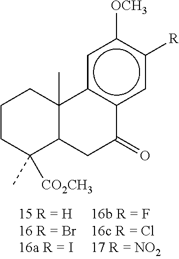 |
| MK18 MK19 MK19a MK19b MK19c | 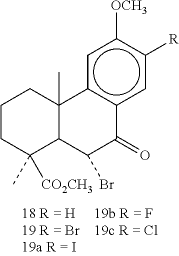 |
| MK21 MK21a MK21b MK22 MK22a MK22b MK20 MK20a | 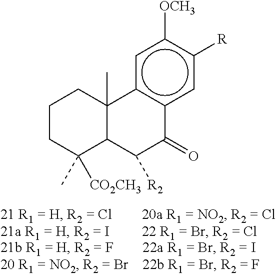 |
| MK18 MK19 | 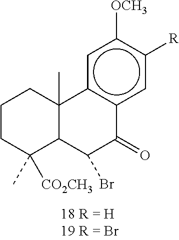 |
| MK12 | 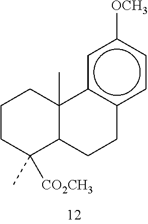 |
| MK27 | 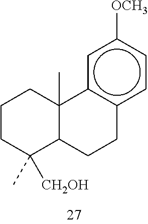 |
| MK28 | 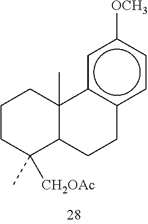 |
| MK34 | 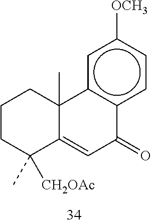 |
| MK29 | 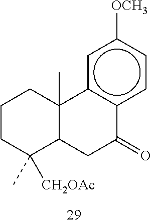 |
| MK30 | 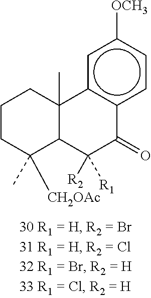 |

The foregoing podocarpic acid analogs and/or derivatives are illustrative and non-limiting. Using the teachings provided herein numerous other podocarpic acid analogs and/or derivatives will be available to one of skill in the art.

The TRPA1 activators useful in the methods described herein are not limited to podocarpic acid or analogs and/or derivatives thereof. Numerous other TRPA1 activators are known to those of skill in the art. Such activators include, but are not limited to tiglic aldehyde, cuminaldehyde, cinnamaldehyde, mustard oil, wasabi, allyl isothiocyanate, and compositions described in PCT Publication WO2014129238 A1 (PCT/JP2014/050763) which is incorporated herein by reference for the TRPA1 activator compounds described therein.

Similarly U.S. Patent Pub. No. 2011/0009379 (which is incorporated herein by reference for the indolinone compounds described therein) discloses indolinone compounds that are TRPA1 channel activators. Illustrative compounds include, but are not limited to a compound according to Formula I or a pharmaceutical acceptable salt thereof:

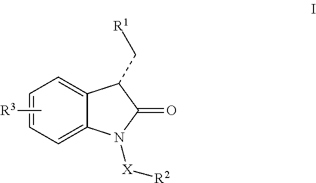

where, R¹ is —CO₂H or a biological equivalent thereof, —CO₂—R⁰, —CON(—R⁴)(—R⁵), —CN, —CO-(nitrogen-containing hetero ring which may be substituted with) —R⁰, or nitrogen-containing hetero ring which may be substituted with —R⁰, R⁰ is C₁₋₆ alkyl, R⁴ and R⁵ are the same or different, representing —H, C₁₋₆ alkyl, C₃-8 cycloalkyl, —OH, or —SO₂—C₁₋₆ alkyl, X is C₁₋₁₀ alkylene, or —(C₁₋₁₀ alkylene)-O—, R² is (i) hetero ring, aryl, C₃-8 cycloalkyl or —CO—R⁰, each of which may be substituted with group(s) selected from —O—R⁰, —O—R⁰⁰ -aryl, —CON(—R⁴)(—R⁵), —CO-(nitrogen-containing hetero ring which may be substituted with —R⁰, —CONHSO₂—R⁰, —CONHOH, —NO₂ and —CN, or (ii) —H, or —R⁰, R⁰⁰ is a bond or C₁₋₆ alkylene, R³ is —H, —R⁰, C₁₋₆ alkyl which may be substituted with one or more halogens, halogen, —NO₂, —CN, or —O—R⁰, the dotted line is Z-olefin or E-olefin, or a mixture thereof, provided that, (a) when R¹ is methoxycarbonyl, ethoxycarbonyl, N,N-dimethylaminocarbonyl or N-phenylaminocarbonyl, and —X—R² is methyl, R³ represents a group other than —H, and (b) when R¹ is ethoxycarbonyl, —CO₂H or —CON(CH₃)₂, and —X—R² is benzyl, R³ represents a group other than —H). In certain embodiments le is —CO₂H or a biological equivalent thereof, —CO₂—R⁰, —CON(—R⁴)(—R⁵), —CN, —CO-(nitrogen-containing hetero ring) or nitrogen-containing hetero ring which may be substituted with —R⁰, R⁴ and R⁵ are the same or different, representing —H, or C₁₋₆ alkyl, and R² is (i) hetero ring, aryl, cycloalkyl or —CO—R⁰, each of which may be substituted with group(s) selected from —O—R⁰, —O—R⁰⁰-aryl, —CO₂—R⁰, —CON(—R⁴((—R⁵), —CO-(nitrogen-containing hetero ring), —CONHSO₂—R⁰, —CONHOH, —NO₂ and —CN, or (ii) —H, or —R⁰. In certain embodiments R¹ is —CO₂H, —CON(—R⁴)(—R⁵), —CN, —CO-(nitrogen-containing hetero ring which may be substituted with)—R° or nitrogen-containing hetero ring which may be substituted with —R⁰, R² is (i) hetero ring, aryl or cycloalkyl, each of which may be substituted with group(s) selected from —O—R⁰, —O—R⁰⁰-aryl, —CO₂—R⁰ and —CO₂H, or (ii) —H, and R³ is —H, —R⁰, halogen or —O—R° . In certain embodiments the dotted line in formula I is E-olefin and R¹ is —CO₂H, —CONH₂, —CON(CH₃)₂ or —CO-(cyclic amino which may be substituted with)—R⁰. In certain embodiments R³ is —H, —F or —Cl. In certain embodiments —X—R² is C₄₋₆ alkyl. In certain embodiments —X—R² is 2-methylpropan-1-yl, 2-methylbutan-1-yl, 2,2-dimethylpropan-1-yl, 2-ethylbutan-1-yl, 3-methylbutan-1-yl, or 3-methylpentan-1-yl. In certain embodiments —X—R² is C₃₋₈ cycloalkylmethyl or benzyl in which the benzene ring may be substituted with group(s) selected from the group consisting of —O—R⁰ and —CO₂—R⁰. In certain embodiments —X—R² is cyclopropylmethyl, cyclobutylmethyl, cyclopentylmethyl, cyclohexylmethyl or benzyl. In certain embodiments R¹ is —CO₂H, —CONH₂, or —CON(CH₃)₂. In certain embodiments R₁ is pyrrolidin-1-ylcarbonyl, azetidin-1-ylcarbonyl or morpholin-4-ylcarbonyl.

In certain embodiments the compound comprises a compound or a salt thereof where the compound is selected from the group consisting of is (2E)-[1-(cyclohexylmethyl)-2-oxo-1,2-dihydro-3H-indol-3-ylidene]acetic acid, (2E)-(1-benzyl-5-fluoro-2-oxo-1,2-dihydro-3H-indol-3-ylidene)acetic acid, (2E)-(1-benzyl-7-fluoro-2-oxo-1,2-dihydro-3H-indol-3-ylidene)acetic acid, (2E)-[-(cyclopentylmethyl)-2-oxo-1,2-dihydro-3H-indol-3-ylidene]acetic acid, (2E)-(7-fluoro-1-isobutyl-2-oxo-1,2-dihydro-3H-indol-3-ylidene)acetic acid, (2E)-[1-(cyclopentylmethyl)-7-fluoro-2-oxo-1,2-dihydro-3H-indol-3-ylidene]acetic acid, (2E)-(7-chloro-1-isobutyl-2-oxo-1,2-dihydro-3H-indol-3-ylidene)acetic acid, (2E)-[-(cyclobutylmethyl)-7-fluoro-2-oxo-1,2-dihydro-3H-indol-3-yl-idene]acetic acid, (2E)-[-(cyclopropylmethyl)-7-fluoro-2-oxo-1,2-dihydro-3H-indol-3-ylidene]acetic acid, (2E)-[1-(cyclopentylmethyl)-7-fluoro-2-oxo-1,2-dihydro-3H-indol-3-ylidene]-N,N-dimethylacetamide, (3E)-1-(2-ethyl butyl)-7-fluoro-3-(2-morpholin-4-yl-2-oxoethylidene)-1,3-dihydro-2H-indol-2-one, (2E)-{7-fluoro-1-[(2S)-2-methylbutyl]-2-oxo-1,2-dihydro-3H-indol-3-ylidene}acetic acid, (2E)-[7-fluoro-1-(3-methylbutyl)-2-oxo-1,2-dihydro-3H-indol-3-ylidene]acetic acid, (2E)-2-[1-(cyclohexylmethyl)-2-oxo-1,2-dihydro-3H-indol-3-ylidene]-N,N-dimethylacetamide, (2E)-[1-(cyclopentylmethyl)-2-oxo-1,2-dihydro-3H-indol-3-ylidene]-N,N-dimethylacetamide, (3E)-3-(2-azetidin-1-yl-2-oxoethylidene)-1-(cyclohexylmethyl)-1,3-dihydro-2H-indol-2-one, (3E)-3-(2-azetidin-1-yl-2-oxoethylidene)-1-(cyclopentylmethyl)-1,3-dihydro-2H-indol-2-one, (2E)-[1-(2-ethylbutyl)-2-oxo-1,2-dihydro-3H-indol-3-ylidene]acetic acid, (3E)-1-(2-ethylbutyl)-3-(2-oxo-2-pyrrolidin-1-ylethylidene)-1,3-dihydro-2-H-indol-2-one, (3E)-3-(2-azetidin-1-yl-2-oxoethylidene)-1-(2-ethylbutyl)-1,3-dihydro-2H-indol-2-one, (3E)-3-(2-azetidin-1-yl-2-oxoethylidene)-1-(cyclobutylmethyl)-1,3-dihydro-2H-indol-2-one, and (3E)-1-(cyclobutylmethyl)-3-(2-oxo-2-pyrrolidin-1-ylethylidene)-1,3-dihydro-2H-indol-2-one.

The foregoing TRPA1 activators are illustrative and non-limiting. Using the teachings provided herein numerous other TRPA1 activators will be available for use in the methods described herein.

### Pharmaceutical Formulations.

In certain embodiments one or more active agents described herein (e.g., TRPA1 activators (e.g., podocarpic acid or analogs and/or derivatives of podocarpic acid, indolinones, etc.), or tautomer(s) or stereoisomer(s) thereof, or pharmaceutically acceptable salts, solvates, or clathrates of said TRPA1 activators, or derivatives, analogs, or prodrugs thereof) are administered to a mammal in need thereof, e.g., to a mammal at risk for or suffering from a pathology characterized by formation and/or accumulation of advanced glycation endproducts (AGEs). In certain embodiments, the TRPA1 activators are used for the treatment or prophylaxis of diabetes (or pre-diabetes). In certain embodiments, the TRPA1 activators are used for ameliorating a pathology (e.g., ameliorating one or more symptoms of a pathology) characterized by elevated α-dicarbonyl compounds (e.g., Diabetes, Alzheimer's 's disease, Parkinson's disease, cataract formation, stroke, cardiovascular disease, etc.) or prophylactically slowing or stopping the onset of this pathology. In certain embodiments, the TRPA1 activators are used for reducing the rate of formation and/or the levels of α-dicarbonyl compounds in a mammal. In certain embodiments, the TRPA1 activators are used for reducing the amount of, or slowing or stopping the formation and/or accumulation of, advanced glycation endproducts in a mammal.

The active agent(s) (e.g., podocarpic acid or analogs and/or derivatives of podocarpic acid, indolinones, etc.), or tautomer(s) or stereoisomer(s) thereof, or pharmaceutically acceptable salts, solvates, or clathrates of said TRPA1 activators, or derivatives, analogs, or prodrugs thereof) described herein can be administered in the “native” form or, if desired, in the form of salts, esters, amides, prodrugs, derivatives, and the like, provided the salt, ester, amide, prodrug or derivative is suitable pharmacologically, i.e., effective in the present method(s). Salts, esters, amides, prodrugs and other derivatives of the active agents can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience, and as described above.

For example, a pharmaceutically acceptable salt can be prepared for any of the agent(s) described herein having a functionality capable of forming a salt. A pharmaceutically acceptable salt is any salt that retains the activity of the parent compound and does not impart any deleterious or untoward effect on the subject to which it is administered and in the context in which it is administered.

In various embodiments pharmaceutically acceptable salts may be derived from organic or inorganic bases. The salt may be a mono or polyvalent ion. Of particular interest are the inorganic ions, lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules.

Methods of formulating pharmaceutically active agents as salts, esters, amide, prodrugs, and the like are well known to those of skill in the art. For example, salts can be prepared from the free base using conventional methodology that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include, but are not limited to both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt can be reconverted to the free base by treatment with a suitable base. Certain particularly preferred acid addition salts of the active agents herein include halide salts, such as may be prepared using hydrochloric or hydrobromic acids. Conversely, preparation of basic salts of the active agents of this invention are prepared in a similar manner using a pharmaceutically acceptable base such as sodium hydroxide, potassium hydroxide, ammonium hydroxide, calcium hydroxide, trimethylamine, or the like. Particularly preferred basic salts include alkali metal salts, e.g., the sodium salt, and copper salts.

For the preparation of salt forms of basic drugs, the pKa of the counterion is preferably at least about 2 pH units lower than the pKa of the drug. Similarly, for the preparation of salt forms of acidic drugs, the pKa of the counterion is preferably at least about 2 pH units higher than the pKa of the drug. This permits the counterion to bring the solution's pH to a level lower than the pHₘₐₓ to reach the salt plateau, at which the solubility of salt prevails over the solubility of free acid or base. The generalized rule of difference in pKa units of the ionizable group in the active pharmaceutical ingredient (API) and in the acid or base is meant to make the proton transfer energetically favorable. When the pKa of the API and counterion are not significantly different, a solid complex may form but may rapidly disproportionate (i.e., break down into the individual entities of drug and counterion) in an aqueous environment.

Preferably, the counterion is a pharmaceutically acceptable counterion. Suitable anionic salt forms include, but are not limited to acetate, benzoate, benzylate, bitartrate, bromide, carbonate, chloride, citrate, edetate, edisylate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate (embonate), phosphate and diphosphate, salicylate and disalicylate, stearate, succinate, sulfate, tartrate, tosylate, triethiodide, valerate, and the like, while suitable cationic salt forms include, but are not limited to aluminum, benzathine, calcium, ethylene diamine, lysine, magnesium, meglumine, potassium, procaine, sodium, tromethamine, zinc, and the like.

Preparation of esters typically involves functionalization of hydroxyl and/or carboxyl groups that are present within the molecular structure of the active agent. In certain embodiments, the esters are typically acyl-substituted derivatives of free alcohol groups, i.e., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine.

In various embodiments, the active agents identified herein (e.g., podocarpic acid or analogs and/or derivatives of podocarpic acid, indolinones, etc.), or tautomer(s) or stereoisomer(s) thereof, or pharmaceutically acceptable salts, solvates, or clathrates of said TRPA1 activators) are useful for parenteral administration, topical administration, oral administration, nasal administration (or otherwise inhaled), rectal administration, or local administration, such as by aerosol or transdermally, for prophylactic and/or therapeutic treatment of one or more of the pathologies/indications described herein (e.g., pathologies characterized by the accumulation of advanced glycation endproducts).

In various embodiments the active agents described herein can also be combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the active agents, or excipients or other stabilizers and/or buffers.

Other physiologically acceptable compounds, particularly of use in the preparation of tablets, capsules, gel caps, and the like include, but are not limited to binders, diluent/fillers, disintegrants, lubricants, suspending agents, and the like.

In certain embodiments, to manufacture an oral dosage form (e.g., a tablet), an excipient (e.g., lactose, sucrose, starch, mannitol, etc.), an optional disintegrator (e.g.

calcium carbonate, carboxymethylcellulose calcium, sodium starch glycollate, crospovidone etc.), a binder (e.g. alpha-starch, gum arabic, microcrystalline cellulose, carboxymethylcellulose, polyvinylpyrrolidone, hydroxypropylcellulose, cyclodextrin, etc.), and an optional lubricant (e.g., talc, magnesium stearate, polyethylene glycol 6000, etc.), for instance, are added to the active component or components (e.g., podocarpic acid or analogs and/or derivatives of podocarpic acid, indolinones, etc.), or tautomer(s) or stereoisomer(s) thereof, or pharmaceutically acceptable salts, solvates, or clathrates of said TRPA1 activators, or derivatives, analogs, or prodrugs thereof) and the resulting composition is compressed. Where necessary the compressed product is coated, e.g., using known methods for masking the taste or for enteric dissolution or sustained release. Suitable coating materials include, but are not limited to ethyl-cellulose, hydroxymethylcellulose, POLYOX®yethylene glycol, cellulose acetate phthalate, hydroxypropylmethylcellulose phthalate, and Eudragit (Rohm & Haas, Germany; methacrylic-acrylic copolymer).

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physiochemical characteristics of the active agent(s).

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectibles, implantable sustained-release formulations, mucoadherent films, topical varnishes, lipid complexes, etc.

Pharmaceutical compositions comprising the active agents described herein (e.g., podocarpic acid or analogs and/or derivatives of podocarpic acid, indolinones, etc.), or tautomer(s) or stereoisomer(s) thereof, or pharmaceutically acceptable salts, solvates, or clathrates of said TRPA1 activators, or derivatives, analogs, or prodrugs thereof) can be manufactured by means of conventional mixing, dissolving, granulating, dragee-making, levigating, emulsifying, encapsulating, entrapping or lyophilizing processes. Pharmaceutical compositions can be formulated in a conventional manner using one or more physiologically acceptable carriers, diluents, excipients or auxiliaries that facilitate processing of the active agent(s) into preparations that can be used pharmaceutically. Proper formulation is dependent upon the route of administration chosen.

In certain embodiments, the active agents described herein are formulated for oral administration. For oral administration, suitable formulations can be readily formulated by combining the active agent(s) with pharmaceutically acceptable carriers suitable for oral delivery well known in the art. Such carriers enable the active agent(s) described herein to be formulated as tablets, pills, dragees, caplets, lizenges, gelcaps, capsules, liquids, gels, syrups, slurries, suspensions and the like, for oral ingestion by a patient to be treated. For oral solid formulations such as, for example, powders, capsules and tablets, suitable excipients can include fillers such as sugars (e.g., lactose, sucrose, mannitol and sorbitol), cellulose preparations (e.g., maize starch, wheat starch, rice starch, potato starch, gelatin, gum tragacanth, methyl cellulose, hydroxypropylmethyl-cellulose, sodium carboxymethylcellulose), synthetic polymers (e.g., polyvinylpyrrolidone (PVP)), granulating agents; and binding agents. If desired, disintegrating agents may be added, such as the cross-linked polyvinylpyrrolidone, agar, or alginic acid or a salt thereof such as sodium alginate. If desired, solid dosage forms may be sugar-coated or enteric-coated using standard techniques. The preparation of enteric-coated particles is disclosed for example in U.S. Pat. Nos. 4,786,505 and 4,853,230.

For administration by inhalation, the active agent(s) are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator may be formulated containing a powder mix of the compound and a suitable powder base such as lactose or starch.

In various embodiments the active agent(s) can be formulated in rectal or vaginal compositions such as suppositories or retention enemas, e.g., containing conventional suppository bases such as cocoa butter or other glycerides. Methods of formulating active agents for rectal or vaginal delivery are well known to those of skill in the art (see, e.g., Allen (2007) Suppositories, Pharmaceutical Press) and typically involve combining the active agents with a suitable base (e.g., hydrophilic (PEG), lipophilic materials such as cocoa butter or Witepsol W45, amphiphilic materials such as Suppocire AP and polyglycolized glyceride, and the like). The base is selected and compounded for a desired melting/delivery profile.

For topical administration the active agent(s) described herein (e.g., podocarpic acid or analogs and/or derivatives of podocarpic acid, indolinones, etc.), or tautomer(s) or stereoisomer(s) thereof, or pharmaceutically acceptable salts, solvates, or clathrates of said TRPA1 activators) can be formulated as solutions, gels, ointments, creams, suspensions, and the like as are well-known in the art.

In certain embodiments the active agents described herein are formulated for systemic administration (e.g., as an injectable) in accordance with standard methods well known to those of skill in the art. Systemic formulations include, but are not limited to, those designed for administration by injection, e.g. subcutaneous, intravenous, intramuscular, intrathecal or intraperitoneal injection, as well as those designed for transdermal, transmucosal oral or pulmonary administration. For injection, the active agents described herein can be formulated in aqueous solutions, preferably in physiologically compatible buffers such as Hanks solution, Ringer's solution, or physiological saline buffer and/or in certain emulsion formulations. The solution(s) can contain formulatory agents such as suspending, stabilizing and/or dispersing agents. In certain embodiments the active agent(s) can be provided in powder form for constitution with a suitable vehicle, e.g., sterile pyrogen-free water, before use. For transmucosal administration, and/or for blood/brain barrier passage, penetrants appropriate to the barrier to be permeated can be used in the formulation. Such penetrants are generally known in the art. Injectable formulations and inhalable formulations are generally provided as a sterile or substantially sterile formulation.

In addition to the formulations described previously, the active agent(s) may also be formulated as a depot preparations. Such long acting formulations can be administered by implantation (for example subcutaneously or intramuscularly) or by intramuscular injection. Thus, for example, the active agent(s) may be formulated with suitable polymeric or hydrophobic materials (for example as an emulsion in an acceptable oil) or ion exchange resins, or as sparingly soluble derivatives, for example, as a sparingly soluble salt.

In certain embodiments the active agent(s) described herein can also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one illustrative embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

Alternatively, other pharmaceutical delivery systems can be employed. For example, liposomes, emulsions, and microemulsions/nanoemulsions are well known examples of delivery vehicles that may be used to protect and deliver pharmaceutically active compounds. Certain organic solvents such as dimethylsulfoxide also can be employed, although usually at the cost of greater toxicity.

In certain embodiments the active agent(s) described herein (e.g., podocarpic acid or analogs and/or derivatives of podocarpic acid, indolinones, etc.), or tautomer(s) or stereoisomer(s) thereof, or pharmaceutically acceptable salts, solvates, or clathrates of said TRPA1 activators) are formulated in a nanoemulsion. Nanoemulsions include, but are not limited to oil in water (O/W) nanoemulsions, and water in oil (W/O) nanoemulsions. Nanoemulsions can be defined as emulsions with mean droplet diameters ranging from about 20 to about 1000 nm. Usually, the average droplet size is between about 20 nm or 50 nm and about 500 nm. The terms sub-micron emulsion (SME) and mini-emulsion are used as synonyms.

Illustrative oil in water (O/W) nanoemulsions include, but are not limited to: Surfactant micelles—micelles composed of small molecules surfactants or detergents (e.g., SDS/PBS/2-propanol); Polymer micelles—micelles composed of polymer, copolymer, or block copolymer surfactants (e.g., Pluronic L64/PBS/2-propanol); Blended micelles—micelles in which there is more than one surfactant component or in which one of the liquid phases (generally an alcohol or fatty acid compound) participates in the formation of the micelle (e.g., octanoic acid/PBS/EtOH); Integral micelles—blended micelles in which the active agent(s) serve as an auxiliary surfactant, forming an integral part of the micelle; and Pickering (solid phase) emulsions—emulsions in which the active agent(s) are associated with the exterior of a solid nanoparticle (e.g., polystyrene nanoparticles/PBS/no oil phase).

Illustrative water in oil (W/O) nanoemulsions include, but are not limited to: Surfactant micelles—micelles composed of small molecules surfactants or detergents (e.g., dioctyl sulfosuccinate/PBS/2-propanol, isopropylmyristate/PBS/2-propanol, etc.); Polymer micelles—micelles composed of polymer, copolymer, or block copolymer surfactants (e.g., PLURONIC® L121/PBS/2-propanol); Blended micelles—micelles in which there is more than one surfactant component or in which one of the liquid phases (generally an alcohol or fatty acid compound) participates in the formation of the micelle (e.g., capric/caprylic diglyceride/PBS/EtOH); Integral micelles—blended micelles in which the active agent(s) serve as an auxiliary surfactant, forming an integral part of the micelle (e.g., active agent/PBS/polypropylene glycol); and Pickering (solid phase) emulsions—emulsions in which the active agent(s) are associated with the exterior of a solid nanoparticle (e.g., chitosan nanoparticles/no aqueous phase/mineral oil).

As indicated above, in certain embodiments the nanoemulsions comprise one or more surfactants or detergents. In some embodiments the surfactant is a non-anionic detergent (e.g., a polysorbate surfactant, a polyoxyethylene ether, etc.). Surfactants that find use in the present invention include, but are not limited to surfactants such as the TWEEN®, TRITON®, and TYLOXAPOL® families of compounds.

In certain embodiments the emulsions further comprise one or more cationic halogen containing compounds, including but not limited to, cetylpyridinium chloride. In still further embodiments, the compositions further comprise one or more compounds that increase the interaction (“interaction enhancers”) of the composition with microorganisms (e.g., chelating agents like ethylenediaminetetraacetic acid, or ethylenebis(oxyethylenenitrilo)tetraacetic acid in a buffer).

In some embodiments, the nanoemulsion further comprises an emulsifying agent to aid in the formation of the emulsion. Emulsifying agents include compounds that aggregate at the oil/water interface to form a kind of continuous membrane that prevents direct contact between two adjacent droplets. Certain embodiments of the present invention feature oil-in-water emulsion compositions that may readily be diluted with water to a desired concentration without impairing their anti-pathogenic properties.

In addition to discrete oil droplets dispersed in an aqueous phase, certain oil-in-water emulsions can also contain other lipid structures, such as small lipid vesicles (e.g., lipid spheres that often consist of several substantially concentric lipid bilayers separated from each other by layers of aqueous phase), micelles (e.g., amphiphilic molecules in small clusters of 50-200 molecules arranged so that the polar head groups face outward toward the aqueous phase and the apolar tails are sequestered inward away from the aqueous phase), or lamellar phases (lipid dispersions in which each particle consists of parallel amphiphilic bilayers separated by thin films of water).

These lipid structures are formed as a result of hydrophobic forces that drive apolar residues (e.g., long hydrocarbon chains) away from water. The above lipid preparations can generally be described as surfactant lipid preparations (SLPs). SLPs are minimally toxic to mucous membranes and are believed to be metabolized within the small intestine (see e.g., Hamouda et al. (1998) J. Infect. Disease 180: 1939).

In certain embodiments the emulsion comprises a discontinuous oil phase distributed in an aqueous phase, a first component comprising an alcohol and/or glycerol, and a second component comprising a surfactant or a halogen-containing compound. The aqueous phase can comprise any type of aqueous phase including, but not limited to, water (e.g., dionized water, distilled water, tap water) and solutions (e.g., phosphate buffered saline solution or other buffer systems). The oil phase can comprise any type of oil including, but not limited to, plant oils (e.g., soybean oil, avocado oil, flaxseed oil, coconut oil, cottonseed oil, squalene oil, olive oil, canola oil, corn oil, rapeseed oil, safflower oil, and sunflower oil), animal oils (e.g., fish oil), flavor oil, water insoluble vitamins, mineral oil, and motor oil. In certain embodiments, the oil phase comprises 30-90 vol % of the oil-in-water emulsion (e.g., constitutes 30-90% of the total volume of the final emulsion), more preferably 50-80%. The formulations need not be limited to particular surfactants, however in certain embodiments, the surfactant is a polysorbate surfactant (e.g., TWEEN 20®, TWEEN 40®, TWEEN 60®, and TWEEN 80®), a pheoxypolyethoxyethanol (e.g., TRITON® X-100, X-301, X-165, X-102, and X-200, and TYLOXAPOL®), or sodium dodecyl sulfate, and the like.

In certain embodiments a halogen-containing component is present. the nature of the halogen-containing compound, in some embodiments the halogen-containing compound comprises a chloride salt (e.g., NaCl, KCl, etc.), a cetylpyridinium halide, a cetyltrimethylammonium halide, a cetyldimethylethylammonium halide, acetyldimethylbenzylammonium halide, a cetyltributylphosphonium halide, dodecyltrimethylammonium halides, tetradecyltrimethylammonium halides, cetylpyridinium chloride, cetyltrimethylammonium chloride, cetylbenzyldimethylammonium chloride, cetylpyridinium bromide, cetyltrimethylammonium bromide, cetyldimethylethylammonium bromide, cetyltributylphosphonium bromide, dodecyltrimethylammonium bromide, tetradecyltrimethylammonium bromide, and the like

In certain embodiments the emulsion comprises a quaternary ammonium compound. Quaternary ammonium compounds include, but are not limited to, N-alkyldimethyl benzyl ammonium saccharinate, 1,3,5-Triazine-1,3,5(2H,4H,6H)-triethanol; 1-Decanaminium, N-decyl-N,N-dimethyl-, chloride (or) Didecyl dimethyl ammonium chloride; 2-(2-(p-(Diisobutyl)cresosxy)ethoxy)ethyl dimethyl benzyl ammonium chloride; 2-(2-(p-(Diisobutyl)phenoxy)ethoxy)ethyl dimethyl benzyl ammonium chloride; alkyl 1 or 3 benzyl-1-(2-hydroxethyl)-2-imidazolinium chloride; alkyl bis(2-hydroxyethyl)benzyl ammonium chloride; alkyl dimethyl benzyl ammonium chloride; alkyl dimethyl 3,4-dichlorobenzyl ammonium chloride (100% C12); alkyl dimethyl 3,4-dichlorobenzyl ammonium chloride (50% C14, 40% C12, 10% C16); alkyl dimethyl 3,4-dichlorobenzyl ammonium chloride (55% C14, 23% C12, 20% C16); alkyl dimethyl benzyl ammonium chloride; alkyl dimethyl benzyl ammonium chloride (100% C14); alkyl dimethyl benzyl ammonium chloride (100% C16); alkyl dimethyl benzyl ammonium chloride (41% C14, 28% C12); alkyl dimethyl benzyl ammonium chloride (47% C12, 18% C14); alkyl dimethyl benzyl ammonium chloride (55% C16, 20% C14); alkyl dimethyl benzyl ammonium chloride (58% C14, 28% C16); alkyl dimethyl benzyl ammonium chloride (60% C14, 25% C12); alkyl dimethyl benzyl ammonium chloride (61% C11, 23% C14); alkyl dimethyl benzyl ammonium chloride (61% C12, 23% C14); alkyl dimethyl benzyl ammonium chloride (65% C12, 25% C14); alkyl dimethyl benzyl ammonium chloride (67% C12, 24% C14); alkyl dimethyl benzyl ammonium chloride (67% C12, 25% C14); alkyl dimethyl benzyl ammonium chloride (90% C14, 5% C12); alkyl dimethyl benzyl ammonium chloride (93% C14, 4% C12); alkyl dimethyl benzyl ammonium chloride (95% C16, 5% C18); alkyl dimethyl benzyl ammonium chloride (and) didecyl dimethyl ammonium chloride; alkyl dimethyl benzyl ammonium chloride (as in fatty acids); alkyl dimethyl benzyl ammonium chloride (C12-C16); alkyl dimethyl benzyl ammonium chloride (C12-C18); alkyl dimethyl benzyl and dialkyl dimethyl ammonium chloride; alkyl dimethyl dimethybenzyl ammonium chloride; alkyl dimethyl ethyl ammonium bromide (90% C14, 5% C16, 5% C12); alkyl dimethyl ethyl ammonium bromide (mixed alkyl and alkenyl groups as in the fatty acids of soybean oil); alkyl dimethyl ethylbenzyl ammonium chloride; alkyl dimethyl ethylbenzyl ammonium chloride (60% C14); alkyl dimethyl isoproylbenzyl ammonium chloride (50% C12, 30% C14, 17% C16, 3% C18); alkyl trimethyl ammonium chloride (58% C18, 40% C16, 1% C14, 1% C12); alkyl trimethyl ammonium chloride (90% C18, 10% C16); alkyldimethyl(ethylbenzyl) ammonium chloride (C12-18); Di-(C8-10)-alkyl dimethyl ammonium chlorides; dialkyl dimethyl ammonium chloride; dialkyl dimethyl ammonium chloride; dialkyl dimethyl ammonium chloride; dialkyl methyl benzyl ammonium chloride; didecyl dimethyl ammonium chloride; diisodecyl dimethyl ammonium chloride; dioctyl dimethyl ammonium chloride; dodecyl bis(2-hydroxyethyl) octyl hydrogen ammonium chloride; dodecyl dimethyl benzyl ammonium chloride; dodecylcarbamoyl methyl dimethyl benzyl ammonium chloride; heptadecyl hydroxyethylimidazolinium chloride; hexahydro-1,3,5-thris(2-hydroxyethyl)-s-triazine; myristalkonium chloride (and) Quat RNIUM 14; N,N-Dimethyl-2-hydroxypropylammonium chloride polymer; n-alkyl dimethyl benzyl ammonium chloride; n-alkyl dimethyl ethylbenzyl ammonium chloride; n-tetradecyl dimethyl benzyl ammonium chloride monohydrate; octyl decyl dimethyl ammonium chloride; octyl dodecyl dimethyl ammonium chloride; octyphenoxyethoxyethyl dimethyl benzyl ammonium chloride; oxydiethylenebis (alkyl dimethyl ammonium chloride); quaternary ammonium compounds, dicoco alkyldimethyl, chloride; trimethoxysily propyl dimethyl octadecyl ammonium chloride; trimethoxysilyl quats, trimethyl dodecylbenzyl ammonium chloride; n-dodecyl dimethyl ethylbenzyl ammonium chloride; n-hexadecyl dimethyl benzyl ammonium chloride; n-tetradecyl dimethyl benzyl ammonium chloride; n-tetradecyl dimethyl ethylbenzyl ammonium chloride; and n-octadecyl dimethyl benzyl ammonium chloride.

Nanoemulsion formulations and methods of making such are well known to those of skill in the art and described for example in U.S. Pat. Nos. 7,476,393, 7,468,402, 7,314,624, 6,998,426, 6,902,737, 6,689,371, 6,541,018, 6,464,990, 6,461,625, 6,419,946, 6,413,527, 6,375,960, 6,335,022, 6,274,150, 6,120,778, 6,039,936, 5,925,341, 5,753,241, 5,698,219, and 5,152,923 and in Fanun et al. (2009) Microemulsions: Properties and Applications (Surfactant Science), CRC Press, Boca Ratan Fla.

In certain embodiments, one or more active agents described herein can be provided as a “concentrate”, e.g., in a storage container (e.g., in a premeasured volume) ready for dilution, or in a soluble capsule ready for addition to a volume of water, alcohol, hydrogen peroxide, or other diluent.

### Administration

In certain embodiments one or more active agents described herein (e.g., podocarpic acid or analogs and/or derivatives of podocarpic acid, indolinones, etc.), or tautomer(s) or stereoisomer(s) thereof, or pharmaceutically acceptable salts, solvates, or clathrates of said TRPA1 activators) are administered to a mammal in need thereof, e.g., to a mammal at risk for or suffering from a pathology characterized by the formation and/or accumulation of advanced glycation endproducts (AGEs). In certain embodiments the active agent(s) are administered to prevent or delay the onset of a pre-diabetic dysfunction, and/or to ameliorate one or more symptoms of a pre-diabetic dysfunction, and/or to prevent or delay the progression of a pre-diabetic condition or to diabetes. In certain embodiments one or more active agent(s) are administered for the treatment of diabetes, e.g., to reduce the severity of the disease, and/or to ameliorate one or more symptoms of the disease, and/or to slow the progression of the disease.

In various embodiments the active agent(s) described herein (e.g., podocarpic acid or analogs and/or derivatives of podocarpic acid, indolinones, etc.), or tautomer(s) or stereoisomer(s) thereof, or pharmaceutically acceptable salts, solvates, or clathrates of said TRPA1 activators, or derivatives, analogs, or prodrugs thereof) can be administered by any of a number of routes. Thus, for example they can be administered orally, parenterally, (intravenously (IV), intramuscularly (IM), depo-IM, subcutaneously (SQ), and depo-SQ), sublingually, intranasally (inhalation), intrathecally, transdermally (e.g., via transdermal patch), topically, ionophoretically or rectally.

In various embodiments the active agent(s) are administered in an amount/dosage regimen sufficient to exert a prophylactically and/or therapeutically useful effect in the absence of undesirable side effects on the subject treated (or with the presence of acceptable levels and/or types of side effects). The specific amount/dosage regimen will vary depending on the weight, gender, age and health of the individual; the formulation, the biochemical nature, bioactivity, bioavailability and the side effects of the particular compound.

In certain embodiments the therapeutically or prophylactically effective amount may be determined empirically by testing the agent(s) in known in vitro and in vivo model systems for the treated disorder. A therapeutically or prophylactically effective dose can be determined by first administering a low dose, and then incrementally increasing until a dose is reached that achieves the desired effect with minimal or no undesired side effects.

In certain embodiments the agents described herein are administered in an effective amount (dosage). In certain embodiments an effective amount is an amount effective for ameliorating a pathology (e.g., ameliorating one or more symptoms of a pathology) characterized by elevated α-dicarbonyl compounds (e.g., Diabetes, Alzheimer's 's disease, Parkinson's disease, cataract formation, stroke, cardiovascular disease, etc.) or prophylactically slowing or stopping the onset or progression of this pathology. In certain embodiments an effective amount is an amount effective for reducing the rate of formation and/or the levels of α-dicarbonyl compounds in a mammal. In certain embodiments an effective amount is an amount effective for reducing the amount of, or slowing or stopping the formation and/or accumulation of, advanced glycation endproducts in a mammal.

In certain embodiments, when administered orally, an administered amount effective amount of the agent(s) described herein ranges from about 0.1 mg/day to about 500 mg/day or about 1,000 mg/day, or from about 0.1 mg/day to about 200 mg/day, for example, from about 1 mg/day to about 100 mg/day, for example, from about 5 mg/day to about 50 mg/day. In some embodiments, the subject is administered the compound at a dose of about 0.05 to about 0.50 mg/kg, for example, about 0.05 mg/kg, 0.10 mg/kg, 0.20 mg/kg, 0.33 mg/kg, 0.50 mg/kg. It is understood that while a patient may be started at one dose, that dose may be varied (increased or decreased, as appropriate) over time as the patient's condition changes. Depending on outcome evaluations, higher doses may be used. For example, in certain embodiments, up to as much as 1000 mg/day can be administered, e.g., 5 mg/day, 10 mg/day, 25 mg/day, 50 mg/day, 100 mg/day, 200 mg/day, 300 mg/day, 400 mg/day, 500 mg/day, 600 mg/day, 700 mg/day, 800 mg/day, 900 mg/day or 1000 mg/day.

In various embodiments, active agent(s) described herein can be administered parenterally, for example, by IV, IM, depo-IM, SC, or depo-SC. In certain embodiments when administered parenterally, a therapeutically effective amount of about 0.5 to about 100 mg/day, preferably from about 5 to about 50 mg daily can be delivered. When a depot formulation is used for injection once a month or once every two weeks, the dose in certain embodiments can be about 0.5 mg/day to about 50 mg/day, or a monthly dose of from about 15 mg to about 1,500 mg.

In various embodiments, the active agent(s) described herein can be administered sublingually. In some embodiments, when given sublingually, the compounds and/or analogs thereof can be given one to four times daily in the amounts described above for IM administration.

In various embodiments, the active agent(s) described herein can be administered intranasally. When given by this route, the appropriate dosage forms are a nasal spray or dry powder, as is known to those skilled in the art. In certain embodiments, the dosage of compound and/or analog thereof for intranasal administration is the amount described above for IM administration.

In various embodiments, the active agent(s) described herein can be administered intrathecally. When given by this route the appropriate dosage form can be a parenteral dosage form as is known to those skilled in the art. In certain embodiments, the dosage of compound and/or analog thereof for intrathecal administration is the amount described above for IM administration.

In certain embodiments, the active agent(s) described herein can be administered topically. When given by this route, the appropriate dosage form is a cream, ointment, or patch. When administered topically, the dosage is from about 1.0 mg/day to about 200 mg/day. Because the amount that can be delivered by a patch is limited, two or more patches may be used. The number and size of the patch is not important as long as a therapeutically effective amount of compound be delivered as is known to those skilled in the art. The compound can be administered rectally by suppository as is known to those skilled in the art. In certain embodiments, when administered by suppository, the therapeutically effective amount is from about 1.0 mg to about 500 mg.

In various embodiments, the active agent(s) described herein can be administered by implants as is known to those skilled in the art. When administering the compound by implant, the therapeutically effective amount is the amount described above for depot administration.

In various embodiments the dosage forms can be administered to the subject 1, 2, 3, or 4 times daily. In certain embodiments it is preferred that the compound be administered either three or fewer times, more preferably once or twice daily. In certain embodiments, it is preferred that the agent(s) be administered in oral dosage form.

It should be apparent to one skilled in the art that the exact dosage and frequency of administration will depend on the particular condition being treated, the severity of the condition being treated, the age, weight, general physical condition of the particular patient, and other medication the individual may be taking as is well known to administering physicians who are skilled in this art.

While the compositions and methods are described herein with respect to use in humans, they are also suitable for animal, e.g., veterinary use. Thus certain organisms (subjects) contemplated herein include, but are not limited to humans, non-human primates, canines, equines, felines, porcines, ungulates, largomorphs, and the like.

The foregoing formulations and administration methods are intended to be illustrative and not limiting. It will be appreciated that, using the teaching provided herein, other suitable formulations and modes of administration can be readily devised.

### Kits.

In various embodiments the active agents described herein (e.g., TRPA1 activators (e.g., podocarpic acid or analogs and/or derivatives of podocarpic acid, indolinones, etc.), or tautomer(s) or stereoisomer(s) thereof, or pharmaceutically acceptable salts, solvates, or clathrates of said TRPA1 activators, or derivatives, analogs, or prodrugs thereof) can be provided in kits. In certain embodiments the kits comprise the active agent(s) described herein enclosed in multiple or single dose containers. In certain embodiments the kits can comprises component parts that can be assembled for use. For example, an active agent in lyophilized form and a suitable diluent may be provided as separated components for combination prior to use. A kit may include an active agent and a second therapeutic agent for co-administration. The active agent and second therapeutic agent may be provided as separate component parts. A kit may include a plurality of containers, each container holding one or more unit dose of the compounds. The containers are preferably adapted for the desired mode of administration, including, but not limited to tablets, gel capsules, sustained-release capsules, and the like for oral administration; depot products, pre-filled syringes, ampules, vials, and the like for parenteral administration; and patches, medipads, creams, and the like for topical administration, e.g., as described herein.

In certain embodiments the kits can further comprise instructional/informational materials. In certain embodiments the informational material(s) indicate that the administering of the compositions can result in adverse reactions including but not limited to allergic reactions such as, for example, anaphylaxis. The informational material can indicate that allergic reactions may exhibit only as mild pruritic rashes or may be severe and include erythroderma, vasculitis, anaphylaxis, Steven-Johnson syndrome, and the like. In certain embodiments the informational material(s) may indicate that anaphylaxis can be fatal and may occur when any foreign substance is introduced into the body. In certain embodiments the informational material may indicate that these allergic reactions can manifest themselves as urticaria or a rash and develop into lethal systemic reactions and can occur soon after exposure such as, for example, within 10 minutes. The informational material can further indicate that an allergic reaction may cause a subject to experience paresthesia, hypotension, laryngeal edema, mental status changes, facial or pharyngeal angioedema, airway obstruction, bronchospasm, urticaria and pruritus, serum sickness, arthritis, allergic nephritis, glomerulonephritis, temporal arthritis, eosinophilia, or a combination thereof.

While the instructional materials typically comprise written or printed materials they are not limited to such. Any medium capable of storing such instructions and communicating them to an end user is contemplated herein. Such media include, but are not limited to electronic storage media (e.g., magnetic discs, tapes, cartridges, chips), optical media (e.g., CD ROM), and the like. Such media may include addresses to internet sites that provide such instructional materials.

In some embodiments, the kits can comprise one or more packaging materials such as, for example, a box, bottle, tube, vial, container, sprayer, insufflator, intravenous (IV.) bag, envelope, and the like; and at least one unit dosage form of an agent comprising active agent(s) described herein and a packaging material. In some embodiments, the kits also include instructions for using the composition as prophylactic, therapeutic, or ameliorative treatment for the disease of concern.

In some embodiments, the articles of manufacture can comprise one or more packaging materials such as, for example, a box, bottle, tube, vial, container, sprayer, insufflator, intravenous (IV.) bag, envelope, and the like; and a first composition comprising at least one unit dosage form of an agent comprising one or more TRPA1 activators within the packaging material.

### EXAMPLES

The following examples are offered to illustrate, but not to limit the claimed invention.

### Example 1

### Conserved TRPA1-Nrf2 Signaling Mediates Reactive Alpha-Dicarbonyl Detoxification Relevant for Diabetic Pathologies

Chronic hyperglycemia leads to diabetic pathologies through the accumulation of reactive α-dicarbonyls (α-DCs), like methylglyoxal. Evolutionarily conserved glyoxalases are responsible for α-DC detoxification; however their core biochemical regulation have remained unclear.

Developing a genetically tractable invertebrate model for studying diabetic complications, enabling rapid discovery is desirable in the field of diabetes. To that end, we have established a Caenorhabditis elegans model for studying complications associated with α-DC buildup that is amenable to high throughput genetic and drug screens. Recent studies have shown that Glol knockdown in non-diabetic mice result in elevated MGO levels and oxidative stress, ultimately causing pathologies reminiscent of diabetic neuropathy and nephropathy (Distler and Palmer (2012) Front. Genet., 3: 250; Giacco et al. (2014) Diabetes, 63: 291-299). Similarly, our model, based on the mutant glod-4 (Morcos et al. (2008) Aging Cell, 7: 260-269), which is a C. elegans ortholog of the mammalian glutathione-dependent glyoxalase, GLO1, exhibits several phenotypes reminiscent of diabetic complications.

C. elegans is an ideal model for understanding complex molecular networks because of the readily available and powerful genetic tools, ease of culture, and relatively short lifespan (Riddle et al. (1997) Introduction to C. elegans . In C elegans II, D. L. Riddle, T. Blumenthal, B. J. Meyer, and J. R. Priess, eds. (Cold Spring Harbor (NY)). In our studies with this model, we show that TRPA1 acts as a conserved sensor for α-DCs and identify several components of an ensuing signaling pathway that triggers α-DC detoxification via Nrf2 activation. We also performed a phenotypic drug screen using C. elegans model, to identify potential candidates for amelioration of neuropathic and age-related complications associated with diabetes. Ultimately, this work exemplifies the utility of invertebrate systems, such as C. elegans in modeling mammalian diseases and facilitating rapid drug discovery.

More particularly, we have established a Caenorhabditis elegans model, based on an impaired glyoxalase (glod-4/GLO1), to broadly study α-DC-related stress. We show that glod-4 animals rapidly exhibit several diabetes-like phenotypes including hyperesthesia, neuronal damage, and early mortality. We further demonstrate TRPA1 as a sensor for α-DCs, conserved between worms and mammals. Moreover, TRPA1 activates Nrf2 via calcium-modulated kinase signaling, ultimately regulating the glutathione-dependent (GLO1) and -independent (DJ1) glyoxalases to detoxify α-DCs. A phenotypic drug-screen using C. elegans identified podocarpic acid as a novel activator of TRPA1 that rescues α-DC-induced pathologies in C. elegans and mammalian cells. We propose that amelioration of α-DC stress represents a viable option to address related pathologies in diabetes and associated neurodegenerative conditions like Alzheimer's, and Parkinson's disease.

### Results.

C. elegans Glyoxalase I Mutant, Plod—4, Recapitulates Phenotypes Reminiscent of Pathologies Associated with Diabetes in a Glucose-dependent Fashion

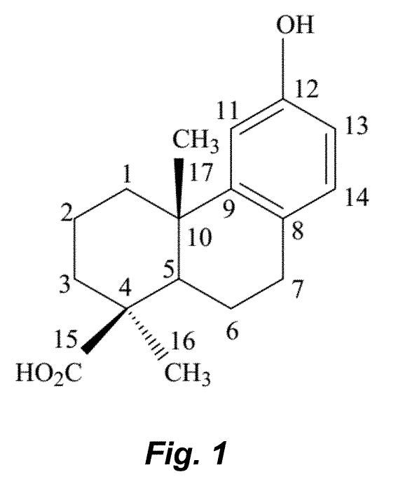

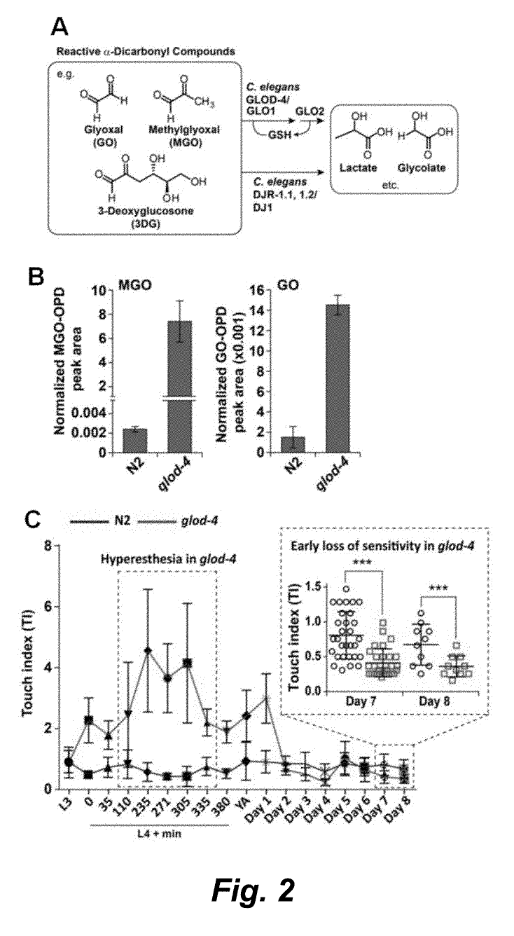

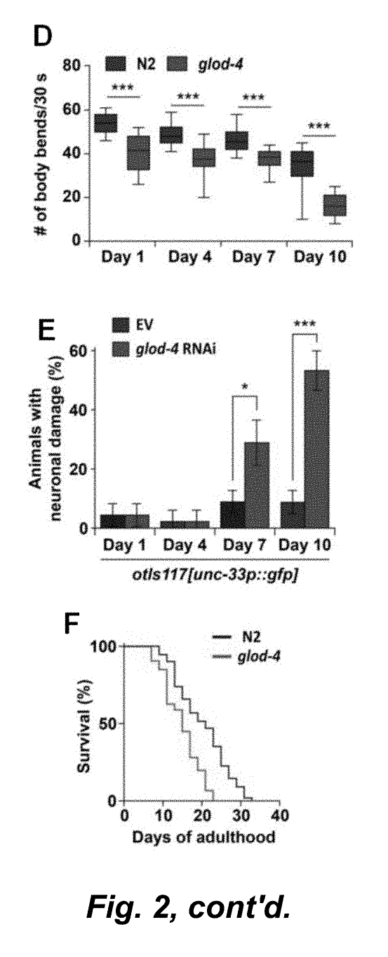

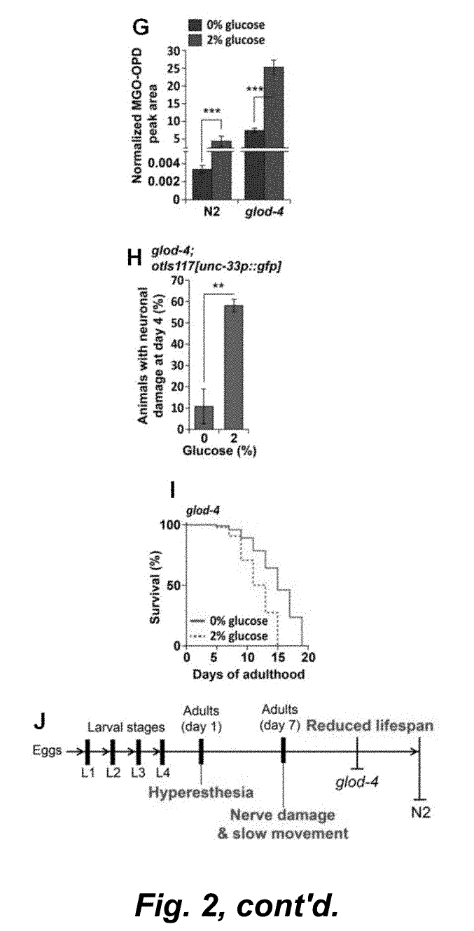

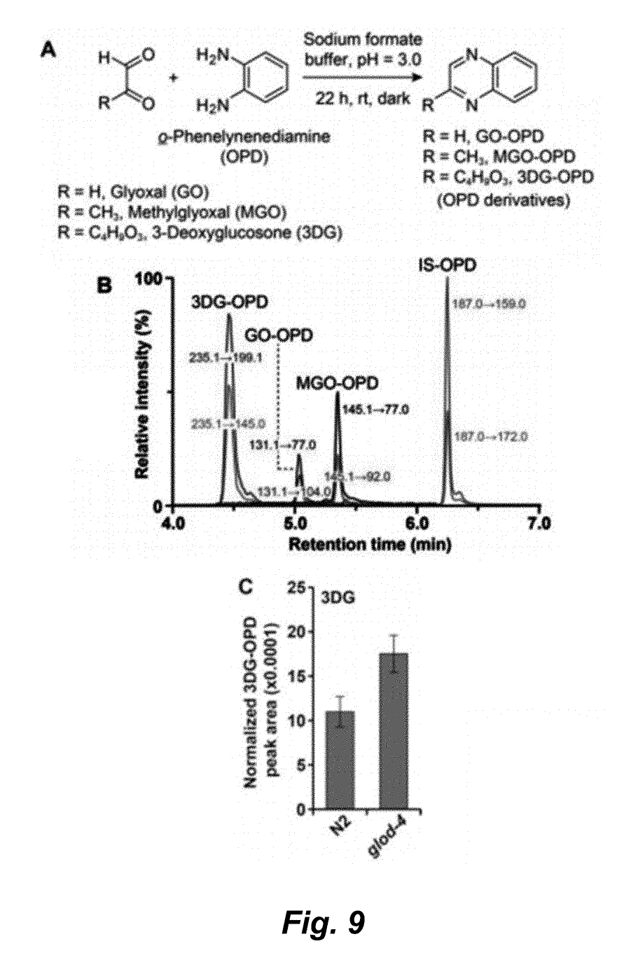

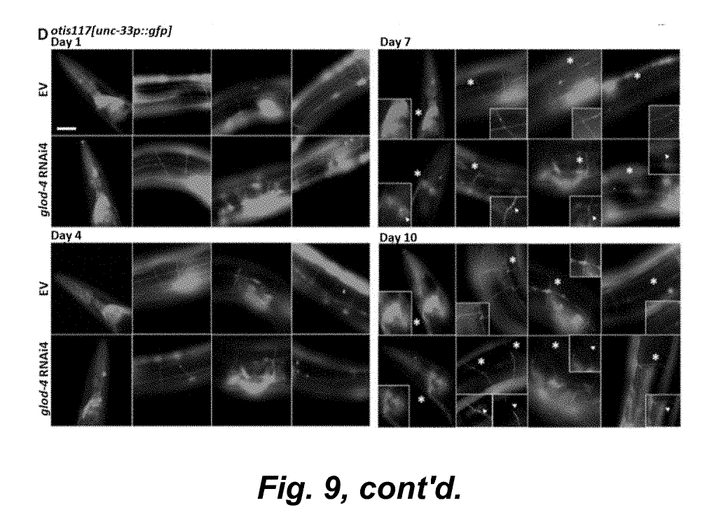

We set out to establish an invertebrate model that accumulates α-DCs and recapitulates phenotypes reminiscent of diabetes-related pathologies. A preliminary LC-MS/MS assay (FIG. 9, panels A and B)-based screen, developed based on previous reports (Henning et al. (2014) J. Biol. Chem., 289: 28676-28688; Rabbani and Thornalley (2014) Nat. Protocol., 9: 1969-1979), revealed that the C. elegans mutant glod-4(gk189) significantly accumulates several α-DCs (GO, MGO, and 3DG) (FIGS. 1B and S1C). Further, glod-4 animals are hypersensitive to touch (hyperesthetic) in an age-dependent fashion (FIG. 2, panel C), which is among the first symptoms experienced by diabetics, who are likely to develop neuropathy (neuronal damage) later in life (Takekuma et al. (2002) Internal. Med., 41: 1124-1129). During early adulthood, glod-4 animals exhibited significantly elevated touch indices (TIs, see Experimental Procedures) as compared to wild-type (N2, Bristol), which leads to a loss of sensitivity to touch later in life (FIG. 2, panel C). Further, as the glod-4 animals aged, we observed reduced motility (FIG. 2, panel D) and neuronal damage (FIGS. 2, panel E, and 9, panel D), as compared to N2. Finally, as is the case with patients suffering from long-term diabetes, glod-4 animals exhibited a significantly shorter lifespan compared to N2 (FIG. 2, panel F).

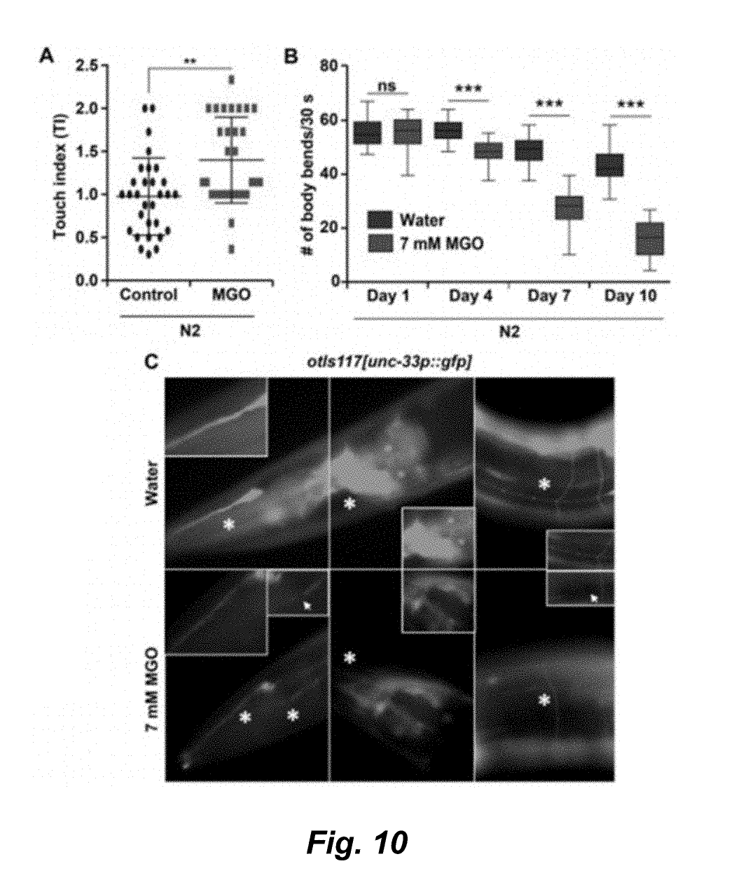

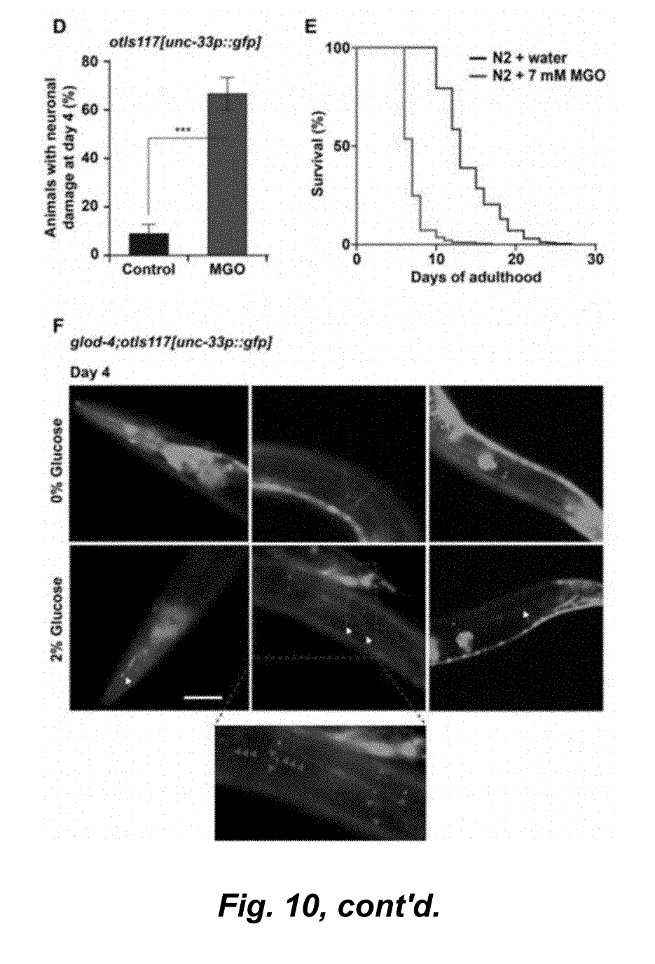

Among the α-DCs assayed, the accumulation of MGO (several thousand fold) was exceedingly more prominent than GO (5-10 fold) or 3DG (1.2-1.5 fold) in glod-4 mutants (FIGS. 2, panel B, and 9, panel C). Hence, we examined if exogenous MGO supplementation could recapitulate glod-4- like phenotypes. MGO treatment on N2 animals leads to hyperesthesia, lower motility, neuronal damage, and early mortality (FIGS. 10, panels A-E), similar to glod-4 mutants. Further, the pathogenic phenotypes in glod-4, i.e., accumulation of MGO, neuronal damage, and early mortality, were exacerbated when the animals were reared on a high glucose (2% glucose) diet (FIG. 2, panels G and I, and FIG. 10, panel F). These findings (FIG. 2, panel J) lay the foundation to utilize glod-4 as a viable model for studying the relation of α-DC stress to hyperesthesia, chronic neuropathic phenotypes, organ damage, and early mortality, relevant to many age-related diseases including diabetes.

The Cation Channel, TRPA-1, Acts as an Upstream Sensor of α-DCs to Trigger SKN-1/Nrf2-mediated Stress Response

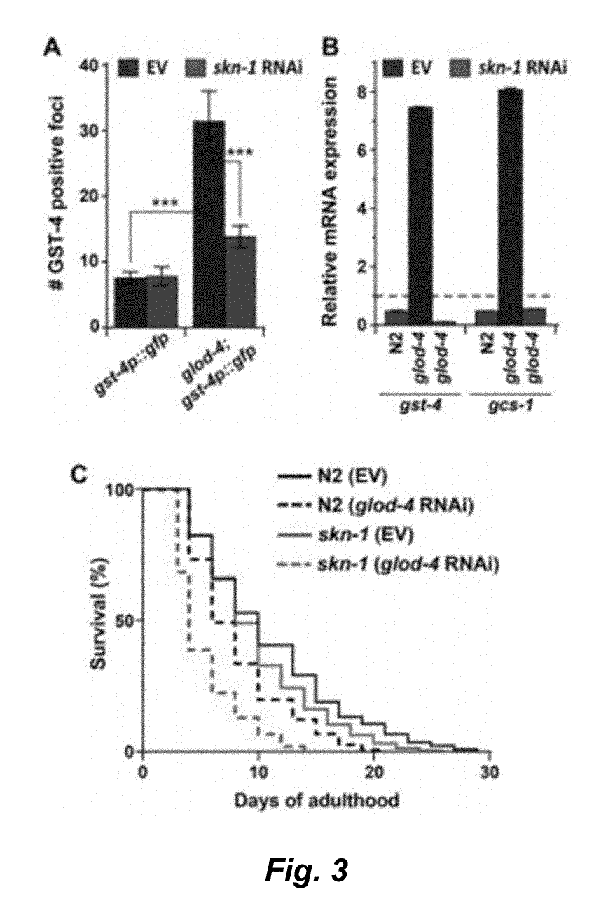

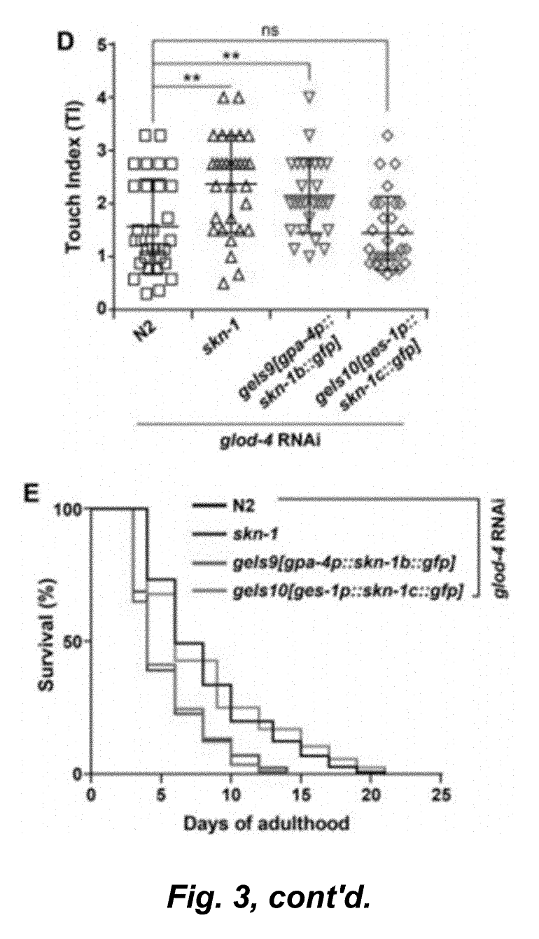

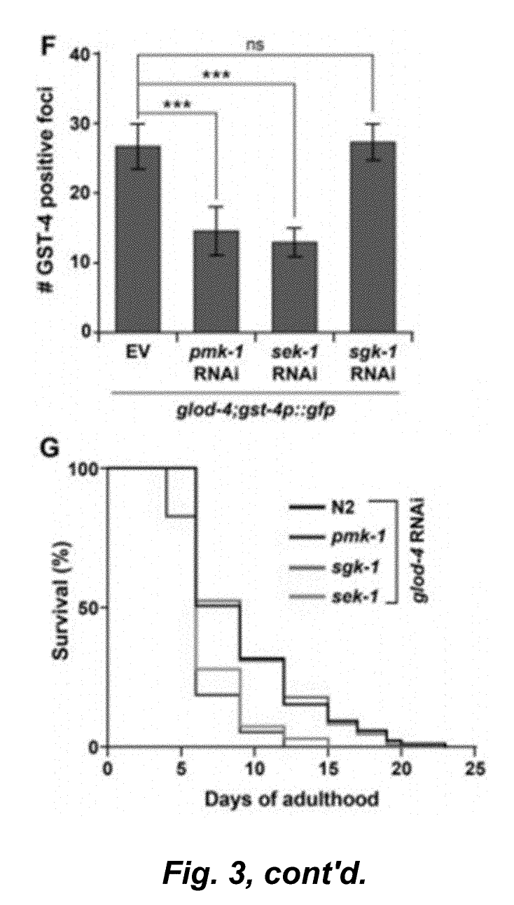

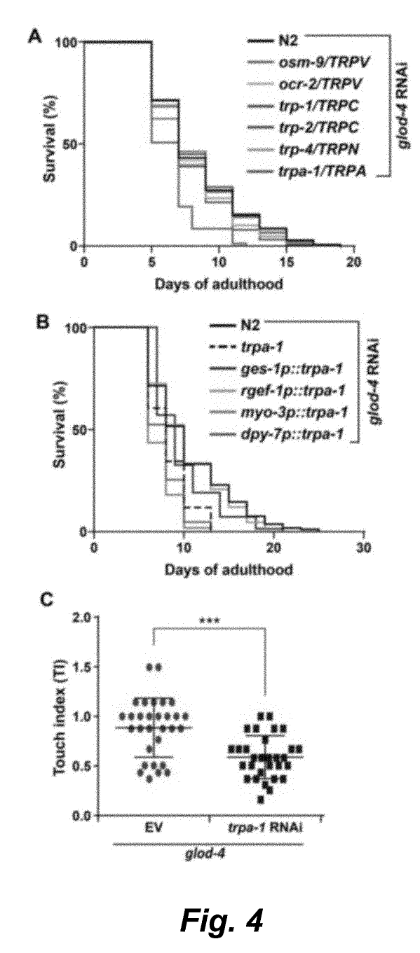

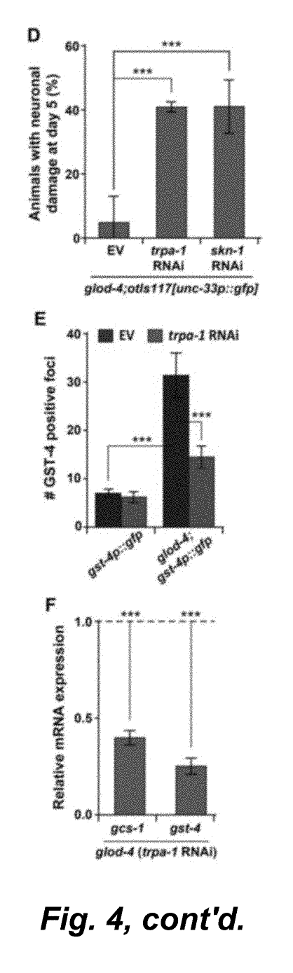

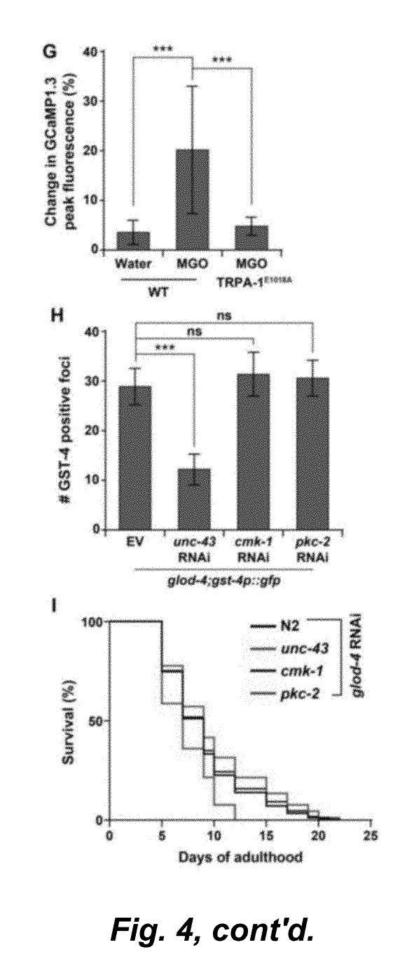

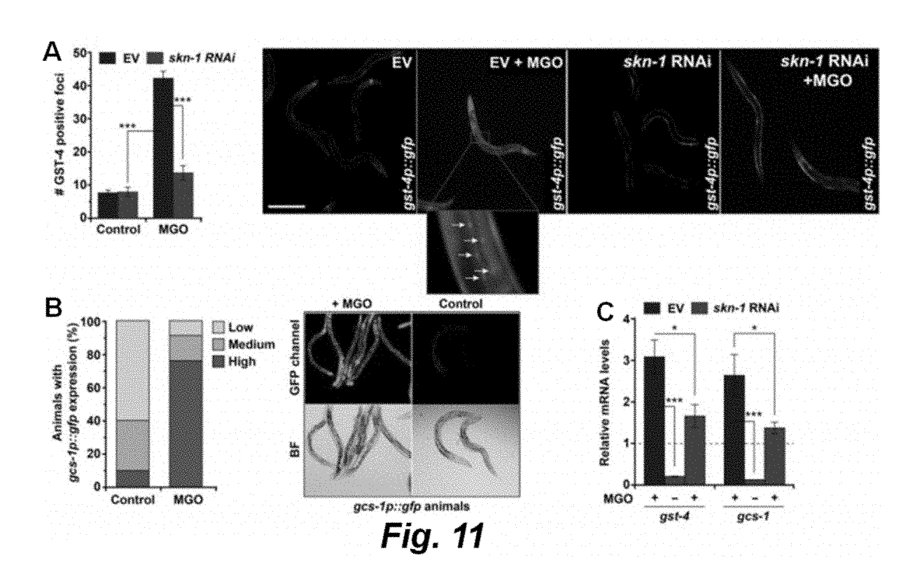

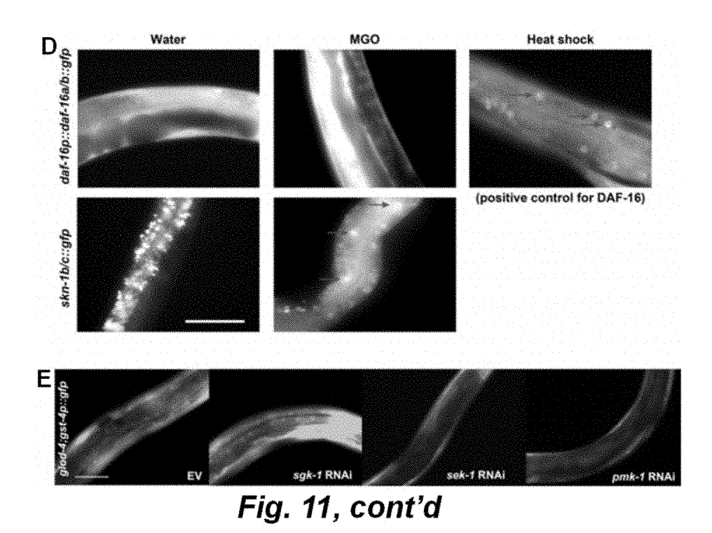

Having established a model, we set out to identify key biochemical pathways that respond to α-DC stress. Among the various conserved components, a protective role of Nrf2 in diabetes-induced oxidative stress have been implicated (Jimenez-Osorio et al. (2015) Clinica Chimica Acta, 448, 182-192; Yang et al. (2015) Scientific Rep. 5: 12377). We first checked if Nrf2 also responds to a-DC stress (as associated with diabetes) and found a significant SKN-1/Nrf2 activation upon glod-4 mutation or exogenous MGO treatment. This was evident from the upregulation of SKN-1 canonical target genes, gst-4 (Glutathione S-Transferase) and gcs-1 (y-glutamyl-cysteine synthase heavy chain), using GFP reporter, as well as RT-PCR-based expression analyses (FIG. 3, panels A-B, and FIG. 11, panels A-C). Further, as reported for various stressors (An and Blackwell (2003) Genes & Dev. 17: 1882-1893; Staab et al. (2013) PLoS Genetics 9: e1003354), we found SKN-1 to also undergo nuclear localization due to MGO stress (FIG. 4, panel D). However, exogenous MGO failed to trigger DAF-16/FOXO nuclear localization, ruling out the direct involvement of insulin/IGF-1 signaling in this process and thus a generic stress response (FIG. 4, panel D). Phenotypically, we observed that the presence of SKN-1 is beneficial for a-DC stress: glod-4 knockdown in skn-1(zu135) mutants, resulted in elevated touch sensitivity and lifespan shortening as compared to N2 (FIG. 3, panels C-E). This protection was achieved tissue-specifically, as only the intestinal SKN-1C isoform expression can rescue the exacerbated touch sensitivity and short lifespan phenotypes of skn-1 animals under glod-4 RNAi (FIGS. 3, panels D and E). Intestinal SKN-1 activation has been previously reported to be dependent on the p38/Mitogen Activated Protein Kinase (MAPK) pathway (An et al. (2005) Proc. Natl. Acad. Sci. USA, 102: 16275-16280). We found that in glod-4;gst-4p::gfp animals, the expression of gst-4 was significantly suppressed by sek-1 or pmk-1 knockdown (C. elegans MAP kinases), but not by knockdown of sgk-1 (FIG. 3, panel F and FIG. 11, panel E), a kinase involved in mediating DAF-2/InR responses in C. elegans (Hertweck et al. (2004) Develop. Cell, 6: 577-588).

Accordingly, glod-4 knockdown significantly reduces the lifespans of pmk-1 and sek-1, but not sgk-1 (FIG. 3, panel G). These results indicate a specific role for p38 MAPK in activating SKN-1 in response to a-DC stress.

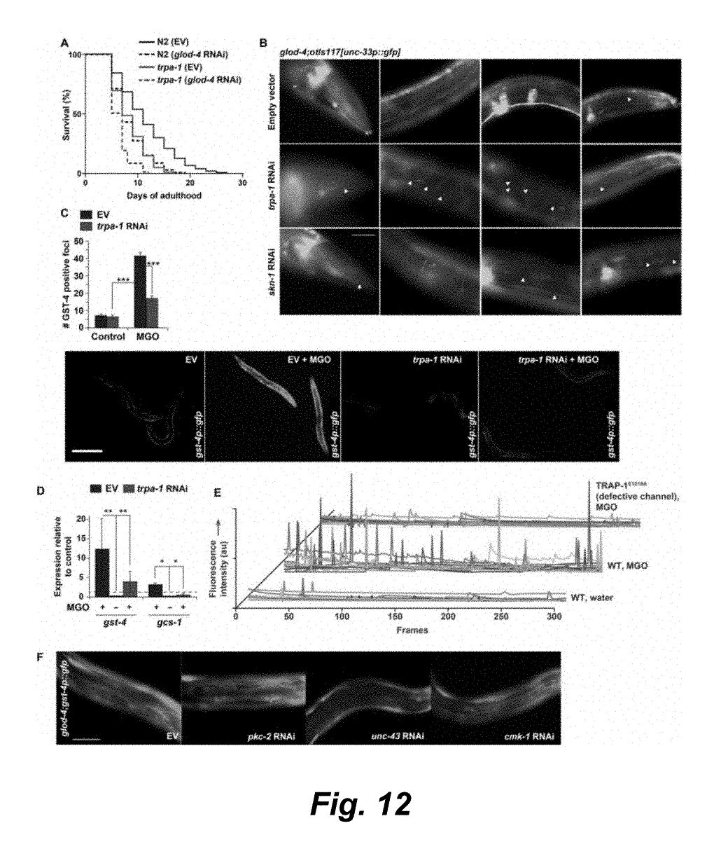

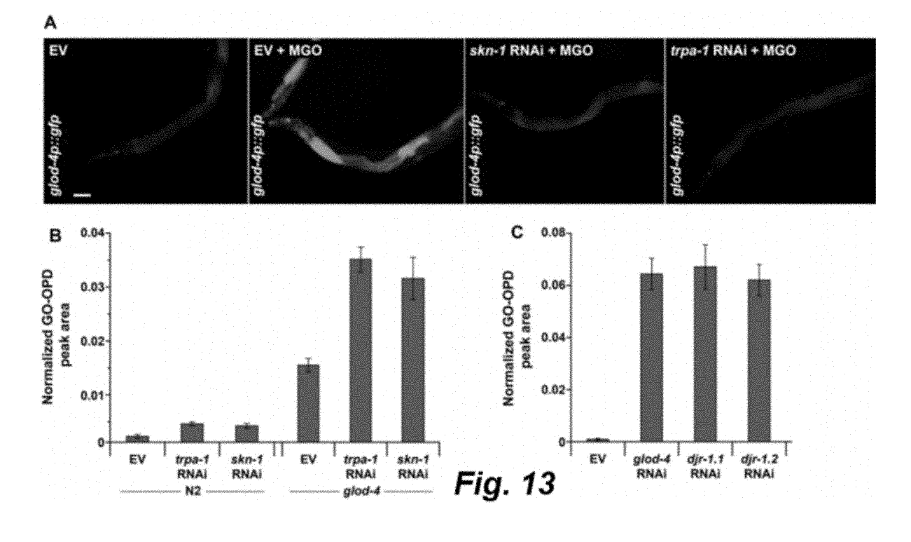

Next, we examined additional upstream components, required for SKN-1 activation under α-DC stress. Several prior studies have implicated ion channels, such as Naᵥ1.8 (Bierhaus et al. (2012) Nat. Med., 18: 926-933) and TRPA1 (Andersson et al. (2013) PloS one, 8: e77986) in MGO-induced nociception. In general, TRP (transient receptor potential) family of ion channels has been implicated in mechanical, thermal, and pain sensation in both vertebrates and invertebrates (Julius (2013) Ann. Rev. Cell Devlop. Biol., 29: 355-384). Among the various TRP channel mutants surveyed, only trpa-1/TRPA1 showed a significant lifespan shortening, compared to N2 under glod-4 RNAi (FIG. 4, panel A and FIG. 12, panel A), suggesting a similar beneficial role of TRPA-1 under a-DC stress. Furthermore, expression of trpa-1 in either intestine or neuron was sufficient to account for the effects of trpa-1 on glod-4 lifespan (FIG. 4, panel B). In apparent contradiction to this protective role of TRPA-1, knockdown of trpa-1 reduced the hypersensitivity to touch response in glod-4 mutants (FIG. 4, panel C). This is not surprising as TRPA-1 is also implicated in mediating C. elegans mechanosensation (Kindt et al. (2007) Nat. Neurosci., 10: 568-577). However, trpa-1 knockdown resulted in an accelerated neuronal damage in glod-4, similar to skn-1 knockdown (FIG. 4, panel D, and 12, panel B), suggesting that the presence of a functional TRPA-1 is ultimately neuroprotective, corroborating the lifespan effects. Interestingly, expression of the SKN-1 reporters, gst-4 and gcs-1, were significantly reduced on trpa-l knockdown both in glod-4 and MGO-treated wild-type animals (FIG. 4, panels E-F, and FIG. 12, panels C and D). This suggests that TRPA-1 may be an upstream sensor for a-DC accumulation, required to trigger a SKN-1-dependent stress response.

Then, we set out to identify the intermediary signaling components that transduce the signal from TRPA-1 to SKN-1. Previous studies have shown that permeability of Ca through TRPA-1 is critical for mediating nociception (Andersson et al. (2013) PloS one, 8: e77986; Xiao et al. (2013) Cell, 152: 806-817). Similarly, using a G-CaMP1.3 sensor, we observed a robust Ca²⁺ response only with a functional TRPA-1 channel with MGO; while control (water) treatment or animals harboring the Ca⁺²-impermeable TRPA-1^{E1018A} channel resulted in basal responses (FIGS. 4, panel G, and 12, panel E). This suggests the specificity of TRPA-1 in modulating a MGO-induced Ca⁺² flux, which cannot be compensated for by any other Ca⁺² channels. Then we asked how this TRPA-1-dependent Ca⁺² flux is transduced to activate SKN-1, a biochemical connection that remained unrecognized. We examined the involvement of candidate Ca⁺²-sensitive kinases that modulate C. elegans behavior and lifespan (Reiner et al. (1999) Nature, 402: 199-203; Robatzek and Thomas (2000) Genetics, 156: 1069-1082; Xiao et al. (2013) Cell, 152: 806-8170. Knockdown of unc-43 (CaMK or Ca²⁺/calmodulin-dependent kinase) reduced expression of the SKN-1 reporter gst-4p::gfp in glod-4 animals, whereas cmk-1 (another CaMK) or pkc-2 (Protein kinase C) had no effect (FIG. 4, panel H, and FIG. 12, panel F). Corroborating this, knockdown of glod-4 significantly shortened lifespan of unc-43(n498,n1186), but not cmk-1(oy21) or pkc-2(ok328) mutants (FIG. 4, panel I), thus specifically implicating the role of UNC-43 in this pathway.

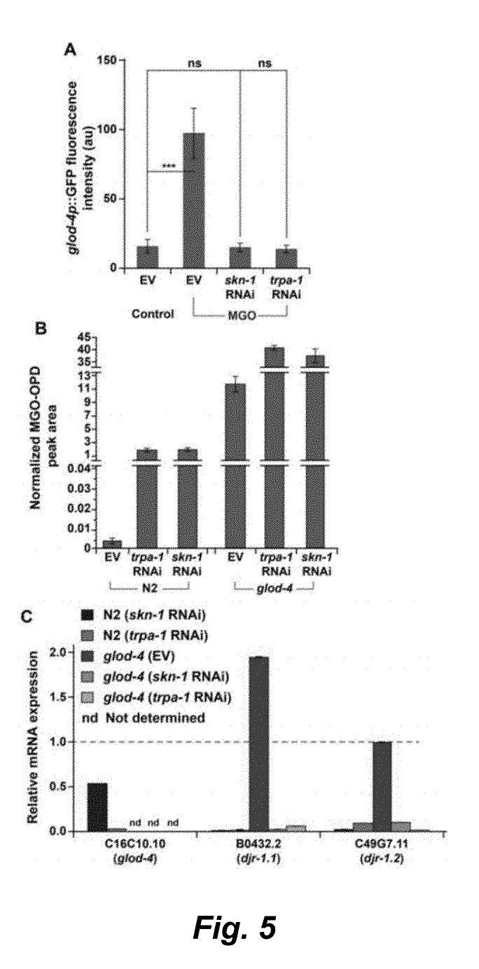

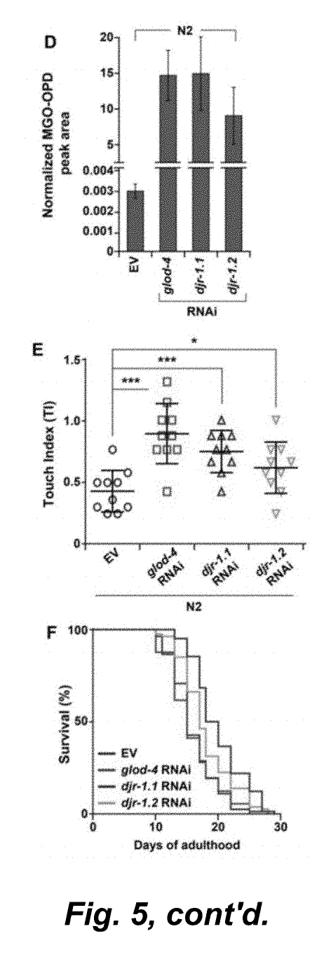

Next, we investigated how this TRPA-1/SKN-1 signaling cascade downstream provides physiological protection under elevated a-DC conditions. We found that the TRPA-1/SKN-1 network regulates the expression of GLOD-4/GLO1, a fundamental a-DC detoxification enzyme: we found that glod-4p::gfp reporter showed a trpa-1- and skn-1 -dependent increase in expression upon MGO treatment (FIG. 5, panel A and 13, panel A). Further, consistent with the exacerbated pathogenic phenotypes, we found that trpa-1 or skn-1 knockdown results in increase in MGO and GO levels, both in N2 and glod-4 (FIG. 5, panel B and 13, panel B). In particular, the increase in MGO and GO due to skn-1 or trpa-1 knockdown in glod-4 background is quite exciting as it suggests the existence of additional GLOD-4- independent TRPA-1/SKN-1-regulated a-DC detoxification pathway(s). We then examined whether these could involve the conserved co-factor-independent glyoxalase enzyme(s) (Lee et al., 2012), DJR-1.1 and -1.2 (human DJ1) and how they may complement GLOD-4 for a-DC detoxification. We found that the expression of the glyoxalases (glod-4, djr-1.1, and djr-1.2) are strongly regulated by trpa-1 and skn-1 (FIG. 5, panel C). Interestingly, while djr-1.1 expression is glod-4-dependent, djr-1.2 expression is not, suggesting a co-option in the trpa-1 /skn-1 -mediated a-DC detoxification network. Further, changes to MGO and GO levels due to djr-.1.1 and djr-1.2 knockdown are comparable to that of glod-4 knockdown (FIGS. 5, panel D and 13, panel C). Accordingly, djr-1.1 and djr-1.2 knockdown result in increased touch sensitivity and reduced lifespan phenotypes similar to glod-4 (FIG. 5, panels E and F).

Leveraging the C. elegans Plod—4 Model to Develop Novel Pharmacological Interventions for Diabetic Complications

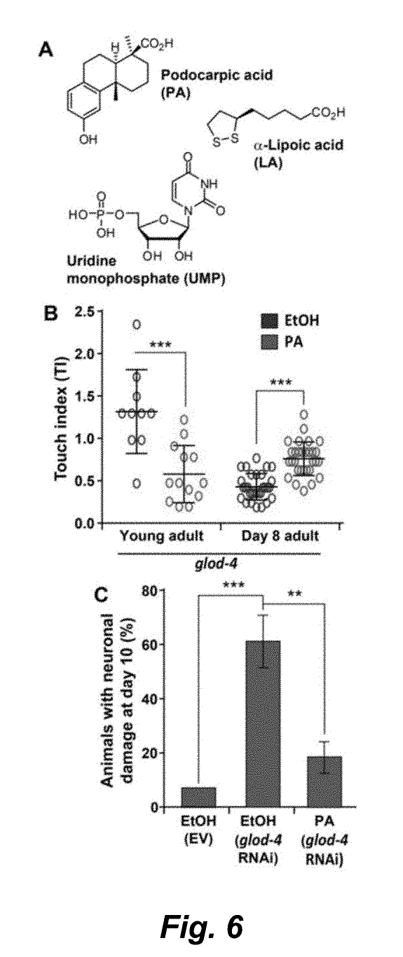

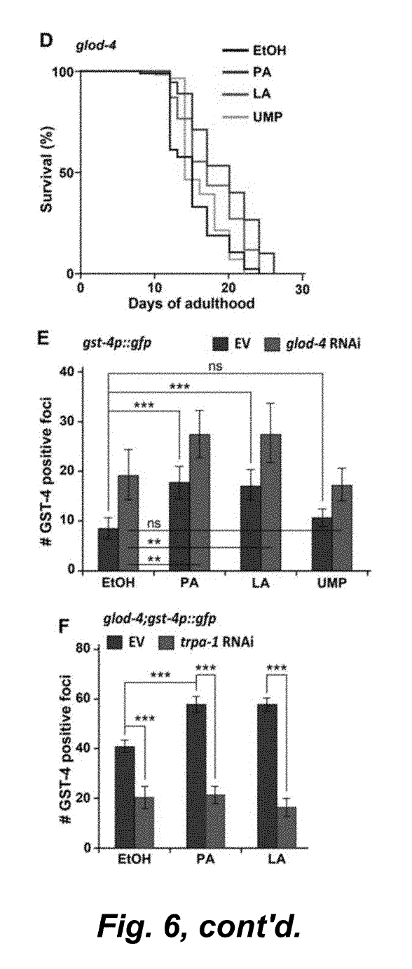

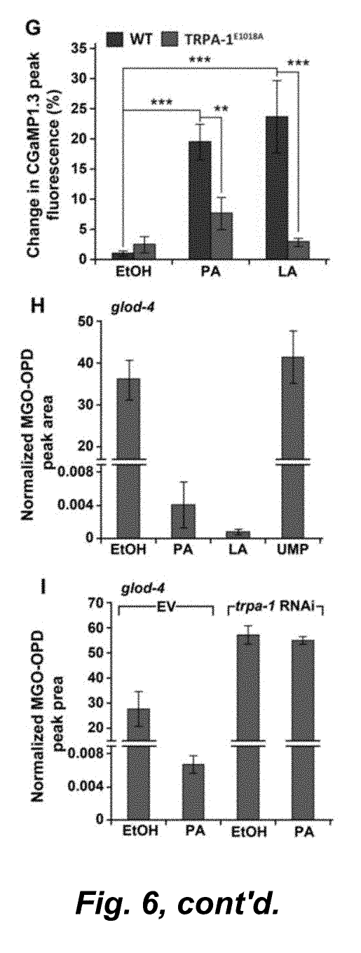

With a firm understanding of the biochemical regulation of α-DC stress, we set out to answer one of the more contemporary and practical questions: can we use the glod-4 model to identify novel therapies for treating diabetic pathologies associated with α-DC stress? Currently, there is a paucity of therapeutics that addresses diabetic complications directly by inhibiting a-DC stress or AGE buildup. Taking advantage of the simplicity and ease of experimental setup in C. elegans (Petrascheck et al. (2007) Nature, 450: 553-556) we carried out a preliminary screen of a library of natural products (TimTec Inc. NPL-640), first to ameliorate the hyperesthesia phenotype exhibited by glod-4 animals. Among compounds that had an all-round positive effect on glod-4 phenotypes, podocarpic acid (FIG. 6, panel A), a natural product isolated from the New Zealand conifer Dacrydium cupressinum (Cui et al. (2008) Bioorganic & Med. Chem. Letts. 18: 5197-5200), featured among the best. Podocarpic acid (PA) was able to alleviate the glod-4 touch sensitivity phenotypes (FIGS. 6, panel B and 14, panel A), i.e., revert the hyperesthesia of glod-4 young adults, as well as lack of touch sensitivity to wild-type levels in day 8 adults. Furthermore, PA treatment was able to prevent the prominent neuronal damages associated with aging glod-4 animals as well their early mortality (FIGS. 6, panels C and D, and 14, panel B).

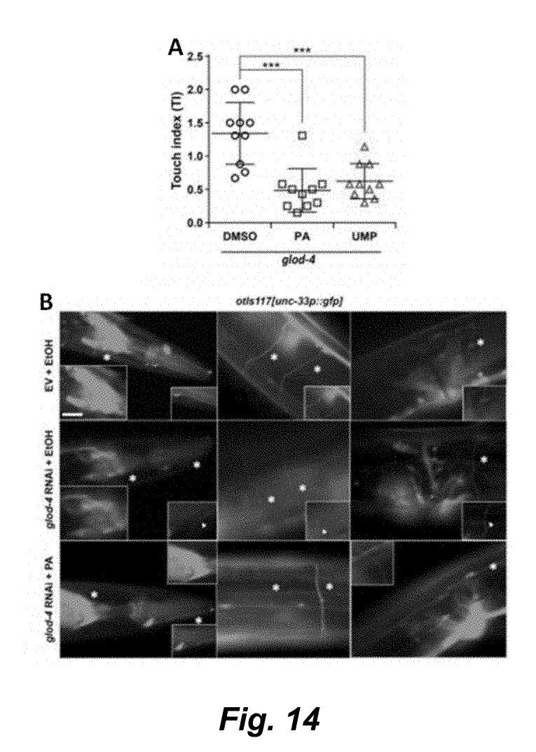

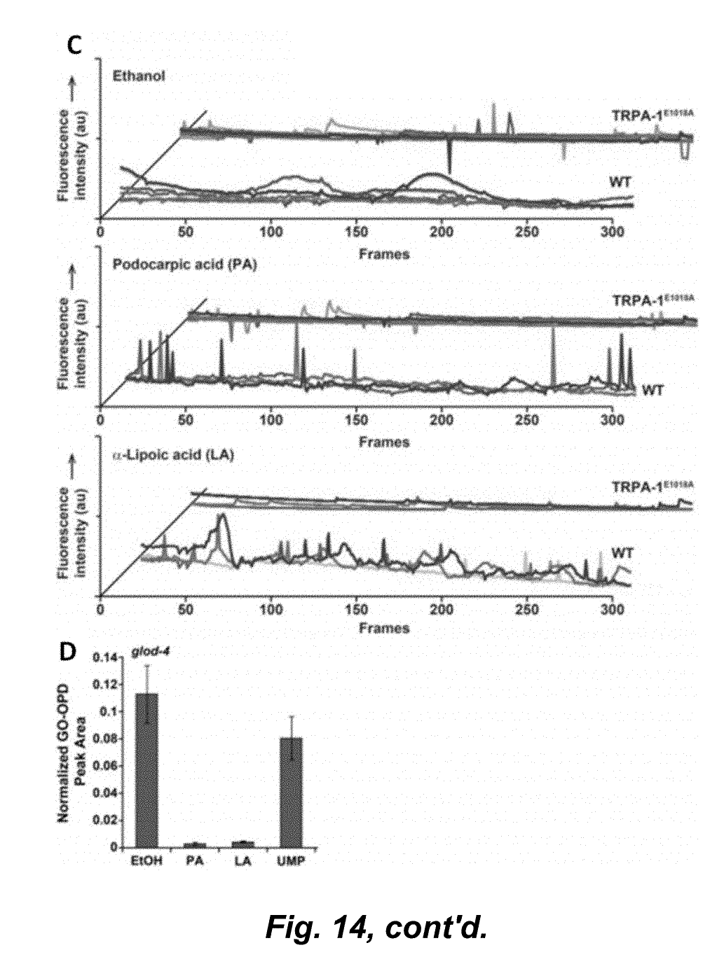

Next, we checked if this new compound utilizes the TRPA-1/SKN-1-controlled a-DC detoxification pathway for ameliorating the pathogenic phenotypes associated with glod-4. We found that PA activates SKN-1 in C. elegans , similar to known Nrf2 activators such as a-lipoic acid (LA) (FIG. 6, panels A and E). LA is currently used as a dietary supplement for diabetic complications (Vallianou et al. (2009) Rev. Diabetic Stud. 6: 230-236) and performed similar to PA in glod-4 lifespan assays (FIG. 6, panel D). Interestingly, we found that the PA or LA-induced SKN-1 activation also requires TRPA-1. When we knocked down trpa-1 in glod-4;gst-4p::gfp animals, expression of gst-4 was significantly reduced to wild-type levels for both PA and LA treatment (FIG. 6, panel F). More importantly, this PA and LA- mediated SKN-1 activation was observed to a similar extent in both N2 and glod-4 backgrounds (FIG. 6, panels E and F). This suggests that TRPA-1/SKN-1 activation via a-DCs and via PA or LA happens largely through distinct mechanisms. Further, PA and LA supplementation results in a robust Ca+² flux, which is significantly reduced when the Ca⁺²-impermeable TRPA-1^{E1018A} channel is present (FIG. 6, panel G and FIG. 14, panel C), suggesting that TRPA-1 activation is key for these drugs' function. Finally, we found that PA and LA are capable of alleviating the pathogenic phenotypes of glod-4 animals by reverting the high endogenous MGO and GO to almost wild-type-like levels (FIGS. 6, panel H and 14, panel D). Consistent with action of PA to be dependent on trpa-1, under trpa-1 knockdown, PA has no effect on MGO levels (FIG. 6, panel I). Thus, our studies suggest that in the absence of glod-4, activation of trpa-1 can ameliorate a-DC stress and downstream damages, perhaps by engaging other targets of SKN-1/Nrf2 such as DJR-1.1 and -1.2. Uridine monophosphate (UMP) was used as a negative control in these assays. While UMP, a hit from our drug screen results in reduced touch sensitivity in young adult animals (FIG. 14, panel A), it does not ameliorate any of the other deleterious phenotypes associated with glod-4, e.g., lifespan shortening or the high a-DC levels (FIG. 6, panels D and H, and FIG. 14, panel D), nor does it activate SKN-1 (FIG. 6, panel E).

Conservation of MGO-induced Neurotoxicity and Its Rescue

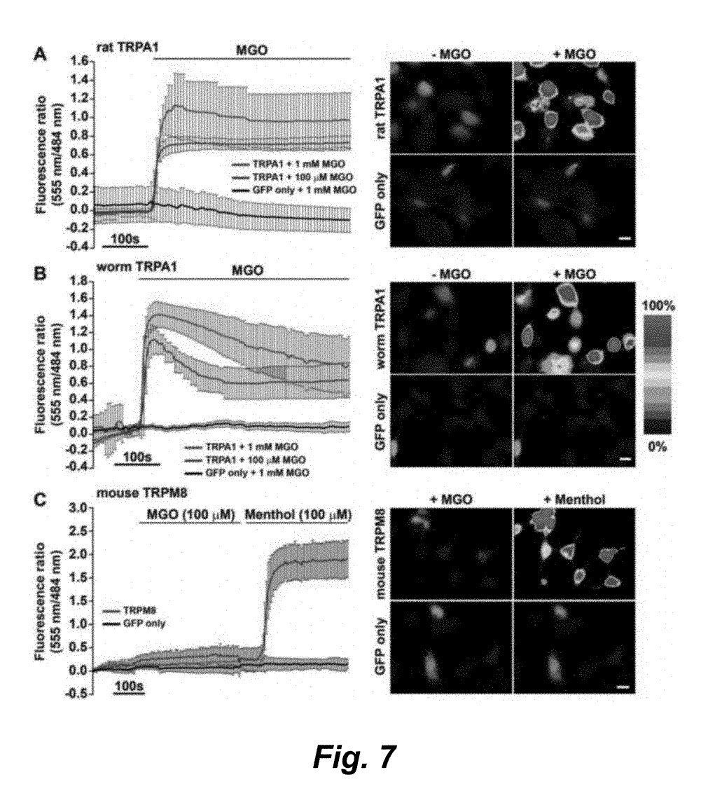

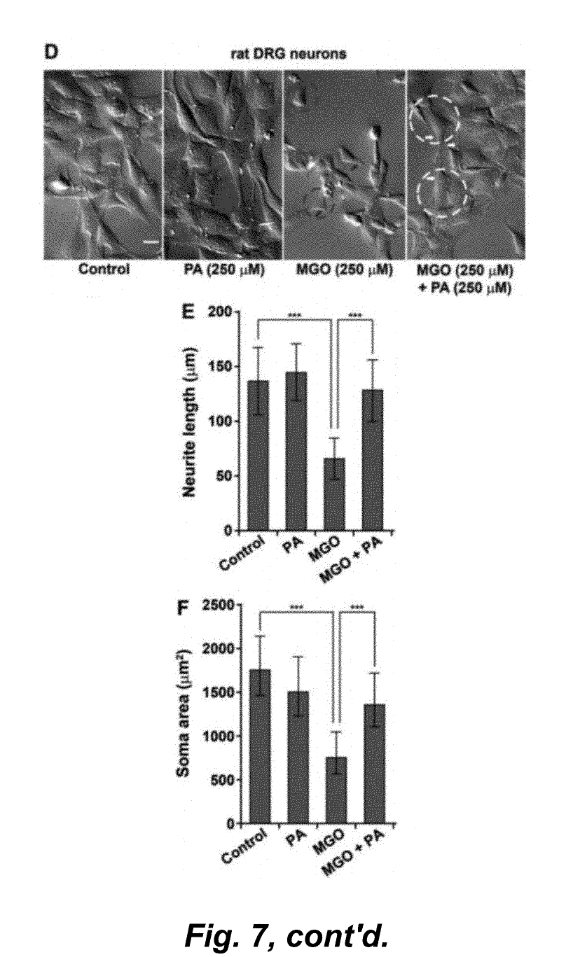

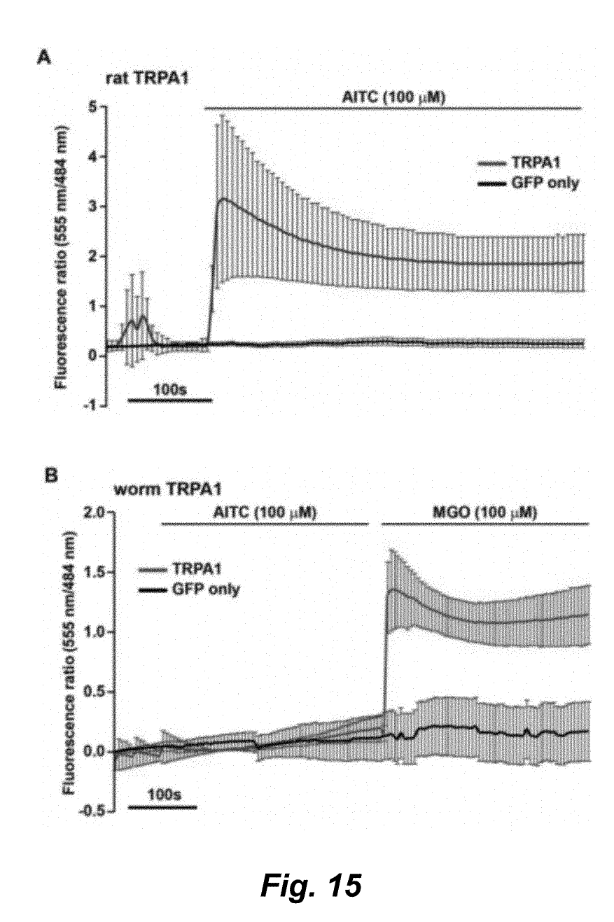

MGO and TRP channels, specifically TRPA1 have been previously implicated in neuropathic pain (Andersson et al. (2013) PloS one, 8: e77986). In C. elegans, we found TRPA-1 to evoke a robust Ca²⁺ flux in response to elevated MGO. Hence, we questioned if this response is conserved across taxa. To that end, we expressed worm and rat TRPA1 in mammalian HEK293T cells and examined MGO-induced Ca⁺² flux response. We found that MGO was able to induce a similar Ca²⁺ flux through both rat and worm TRPA1 channels (FIG. 7, panels A and B). To check if this response is specific to TRPA1, we used native HEK293T cells (expressing GFP sensor only) or HEK293T cells expressing mouse TRPM8, a channel known to result in Ca flux when activated by menthol (Liu and Qin (2005) J. Neurosci., 25: 1674-1681). Our results show that menthol but not MGO, triggers a Ca⁺² flux through the TRPM8 channel (FIG. 7, panel C), whereas neither compounds resulted in Ca⁺² flux in the native HEK293T cells (FIG. 7, panels A-C); suggesting specificity of MGO for TRPA1 activation. In contrast, allyl isothiocyanate (AITC), the compound responsible for the pungent smell in wasabi and mustard oil, known to activate mammalian TRPA1, failed to activate the worm TRPA1 channel (FIG. 15, panels A and B), suggesting that MGO and AITC may have distinct activation mechanisms (vide infra).

Next, we examined whether podocarpic acid (PA) rescues MGO-mediated neurotoxicity. We used the 50B11 cell line (Chen et al. (2007) JPNS, 12: 121-130), an immortalized rat dorsal root ganglion (DRG) neuronal cell line that natively expresses Trpa1 (Andersson et al. (2013) PloS one, 8: e77986) and provides easy visualization of induced neurotoxicity. In differentiated DRG neurons, exposure to MGO resulted in significant neuronal damage evident by shrinkage in cell bodies and significant retraction of neurite outgrowths (FIG. 7, panels D-F). We found that PA was able to ameliorate these MGO-induced neurotoxic phenotypes (FIG. 7, panels D-F). These results further argue the importance of the TRPA1-Nrf2-mediated α-DC detoxification in ameliorating cellular damage and the validity of C. elegans as a model for studying aspects of a-DC-induced pathologies and their relevance in mammalian systems.

### Discussion

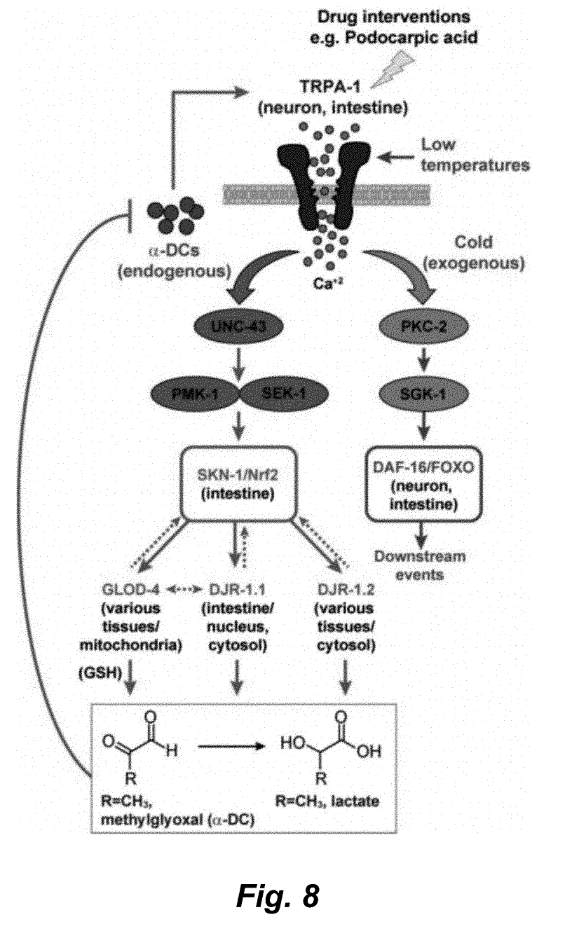

Accumulation of reactive a-DCs, e.g., GO, MGO, 3DG and derived AGEs have been implicated as the root-cause for multiple diabetic complications (Rabbani and Thornalley (2011) Sem. Cell Dev. Biol., 22: 309-317). Additionally, αDC and AGE stress have been associated with neurodegenerative disorders, for which diabetes is an additional risk factor, such as Alzheimer's disease (More et al. (2013) ACS Chem. Neurosci., 4:330-338), Parkinson's disease (Toyoda et al. (2014) Biology open 3: 777-784), and ATTR amyloidosis (da Costa et al. (2011) PloS one, 6: e24850; Gomes et al. (2005) Biochem. J. 385: 339-345). Hence, the C. elegans glod-4 model established in this work may have far-reaching clinical relevance. More importantly, all pathogenic phenotypes in glod-4 mutant occur within a couple of weeks that can take years to develop in humans, significantly fast-tracking biochemical discovery and drug development. As the first step, we have uncovered a conserved regulatory network that mediates endogenous a-DC detoxification (FIG. 8) based on TRPA-1/TRPA1, flux of Ca+² ions relayed by UNC-43/CaMK, and the p38/MAPK kinases SEK-1 and PMK-1 to SKN-1/Nrf2. In response, SKN-1 initiates a transcription program geared towards activation of a multi-faceted a-DC detoxification (FIG. 8). Interestingly, it has been shown that the TRPA-1 mediates the lifespan extension upon cold sensation in C. elegans (Xiao et al. (2013) Cell, 152: 806-817). Even though cold-mediated TRPA-1 activation results in a similar Ca flux, downstream signaling involves distinct kinases PKC-2 and SGK-1 and result in DAF- 16/FOXO activation (Id.). The existence of such a TRPA-1 signaling plasticity is quite fascinating and suggests that other yet-to-be identified signaling components are involved in deciding the course of the organism's response to an endogenous (α-DCs) versus an exogenous (cold) stress. Further, it has been shown that a wide range of TRPA1 agonists such as allyl isothiocyanate (AITC), to activate TRPA1 through covalent modification of specific cysteine residues (Macpherson et al. (2007) Nature, 445: 541-545). Interestingly, C. elegans do not have these specific Cys residues and is therefore refractory to AITC activation (Xiao et al. (2013) Cell, 152: 806-817). However, we observe that the MGO-induced TRPA1 activation is conserved from C. elegans to mammals, suggesting a previously unreported mode of TRPA1 activation by MGO, which could result in the aforementioned plasticity.

Downstream, the activation of the TRPA1-Nrf2 pathway ultimately results in the expression of evolutionarily conserved glutathione-dependent glyoxalase glod-4, and co-factor-independent glyoxalases djr-1.1, and djr-1.2 that convert reactive a-DCs to significantly less reactive metabolites, e.g., MGO to D-lactate (FIG. 8). While Nrf2-dependent regulation of GLO1 has previously been shown in mammalian cell lines (Xue et al. (2012) Biochem. J. 443: 213-222). C. elegans studies that looked for SKN-1 downstream targets did not feature these glyoxalases (Wang et al. (2010) PLoS Genet 6(8). pii: e1001048). Moreover, the mammalian functional ortholog of the glutathione-independent glyoxalase has only recently been identified as DJ1, an enzyme implicated in early onset Parkinson's disease (Lee et al. (2012) Human Mol. Genet. 21: 3215-3225; Toyoda et al. (2014) Biology open 3: 777-784). DJR-1.1 and DJR-1.2 are C. elegans ortholog of DJ1 (Lee et al. (2012) Human Mol. Genet. 21: 3215-3225), and our data shows that along with GLOD-4, both these enzymes are responsible for a-DC detoxification. The redundancy of the glyoxalases perhaps arise to eradicate deleterious reactive a-DCs across the organism, as well as all relevant organelles as each of the glyoxalases are expressed in specific tissues and complementary cellular compartments (FIG. 8) (Lee et al. (2012) Human Mol. Genet. 21: 3215-3225; Morcos et al. (2008) Aging Cell, 7: 260-269). Interestingly, individuals suffering from long-term diabetes are at a higher risk of Parkinson's disease (Santiago and Potashkin (2014) Neurobiol. Dis., 72 Pt A, 84-91). Our results thus take a step closer towards understanding the biochemical ink between diabetes and Parkinson's disease.

Our drug screen and concomitant identification of podocarpic acid suggests that amelioration of a-DC stress represents a viable option for mitigating diabetic complications, which remains under-utilized in diabetes care. Such rapid phenotypic drug screens have the potential to offer several advantages over target-based screens; generally by overcoming problems faced in target-based approaches such as metabolic instability and toxicity due to off-target effects (Pandey and Nichols (2011) Pharmacol. Rev., 63: 411-436). Our results with a-lipoic acid (LA), which is documented to ameliorate diabetic complications in mice and humans (Gomes and Negrato (2014) Diabetology & Metab. Synd. 6: 80), argues for the validity of using the worm model for studying diabetes-like pathologies. However, LA is extensively metabolized in mammals, limiting this compound's potential as a drug Teichert et al. (2003) J. Clin. Pharmacol., 43: 1257-1267. Thus, the identification of podocarpic acid (PA) as a novel TRPA1 activator is quite exciting. The rescue of a-DC-induced phenotypes using PA in C. elegans as well as rat DRG neurons, suggests TRPA1 activators are viable candidates for treating diabetic pathologies, even with a basal Nrf2 activation due to accumulated a-DCs. Additionally, most enzymes featured in our TRPA1-Nrf2 pathway, represent viable druggable targets (FIG. 8), particularly Nrf2 (Suzuki et al. (2013) Trend. Pharmacol. Sci. 34: 340-346). However, attempts at direct Nrf2 activation in vivo has resulted in a multitude of unwanted side-effects (Baraj as et al. (2011) Arterioscl. Thromb. Vascul. Biol. 31: 58-66; DeNicola et al. (2011) Nature, 475: 106-109; Sporn and Liby (2012) Nat. Rev. Canc. 12: 564-571), e.g., promotion of cancerous tumors, development of atheroschlerosis, etc., often outweighing its potential health benefits. Our results suggest that we may be able to circumvent this by indirectly activating Nrf2 via TRPA1. Future studies in mammalian in vivo models will corroborate the utility of such indirect Nrf2 activation and its clinical significance. However, our results contrast some previous findings that suggest TRPA1 channel antagonists in the amelioration of hypersesthesia (Wei et al. (2009) Anesthesiol. 111: 147-154. As noted in other prior publications, results with such antagonist treatment should be interpreted with caution (Andersson et al. (2013) PloS one, 8: e77986), as there may be confounding off-target effects of these compounds. Moreover, while TRPA1 antagonism can provide temporary relief by numbing neuropathic pain, our results show that the presence of a functional TRPA1 is ultimately neuroprotective, beneficial to organismal healthspan. Thus, drug-induced TRPA1 activation is a viable strategy for ameliorating a-DC stress as observed in diabetes, and neurodegenerative conditions such as Parkinson's and Alzheimer's disease.

### Experimental Procedures.

Growth and Maintenance

Worms were cultured at 20° C. for at least two generations under standard growth conditions on 5× Escherichia coli OP50-1 bacterial strain (cultured overnight at 20° C. at 220 rpm) before using for respective experiments (Stiernagle (2006) Maintenance of C. elegans . WormBook, 1-11) and allowed to grow overnight. For feeding RNAi bacteria, synchronized L1 larvae were transferred to NGM plates containing 1 mM of isopropyl P-D-1- thiogalactopyranoside/IPTG (referred to as RNAi plates) seeded with 20× concentrated HT1115 bacteria (cultured overnight at 20° C. at 220 rpm), carrying desired plasmid for RNAi of a specific gene or bacteria carrying empty vector pL4440 as control and allowed to grow on plates for 48 h. For drug assays, synchronized L4 larvae were transferred to either 60 mm NGM plates (with or without IPTG), freshly seeded with 5×E. coli OP50-1 or 20× HT1115 RNAi bacteria. Before seeding, the desired drug (or vehicle control) was mixed with the bacteria. Final drug concentrations were calculated considering the total volume of media and bacteria seeded on the NGM plates.

Note: For glod-4 animals, we found that the pathogenic phenotypes discussed in this paper are contingent on strictly maintaining an ad lib feeding regimen. Hence, care was taken to not to allow the animals to starve by maintaining a low worm to bacteria ratio and transferring to fresh plates frequently (at least once every two days).

Lifespan Assay

Life span assays were performed in Thermo Scientific Precision incubators at 20° C. After alkaline hypochlorite treatment, synchronized L1 animals were either placed onto NGM plates seeded with 5× concentrated E. coli OP50-1 (cultured on Lysogeny Broth/LB overnight) or on RNAi plates supplemented with 20×HT1115 RNAi bacteria. Post-L4 stage of development, all lifespan assays were performed using FUdR (5-fluoro-2 deoxyuridine) plates to inhibit development and growth of progeny. Every two days, animals were transferred on to new 60 mm NGM or RNAi plates freshly seeded with OP50-1 or HT1115 bacteria (with or without drug), respectively. 90-120 animals were considered for each lifespan experiment. Animal viability was assessed visually or with gentle prodding on the head. Animals were censored in the event of internal hatching of the larvae, body rupture, or crawling of larvae from the plates.

Touch Assay

Mechanosensory responsiveness to gentle touch was adapted from a previously described protocol (Hobert et al. (1999) J. Cell Biol. 144: 45-57). Briefly, each animal was touched alternately in the head region and in the tail with an eyelash. Typically, an omega turn or diversion in head direction resulting from anterior touch was counted as a positive response. Posterior touch response (not counted towards positive response) functioned towards resetting of the response to anterior touch. Touch index (TI) scores were generated by dividing the total number of positive responses over the number of negative responses per animal.

Assays for Assessing Neuronal Damage

Neuronal damage was assayed using pan-neuronal GFP reporter strain under different conditions and at different days of adulthood. Animals were paralyzed using freshly prepared 5 mM levamisole in M9 buffer and mounted on 2% agar pads under glass coverslips. Neuronal damage was visually inspected under an upright Olympus BX51 compound microscope coupled with a Hamatsu Ocra ER digital camera. Images were acquired under 40× objective. Neuronal deterioration was examined and characterized by loss of fluorescent intensity of nerve ring, neuronal waviness, and thinning and fragmentation of axons and neuronal commissures. Quantification and imaging of animals harboring damage was performed using the Image J™ software (//imagei.nih.gov/ii/).

Growth, Maintenance, Drug Administration, and Imaging of 50B11 Cell Line

50B11 cells (immortalized rat DRG neuronal cells) maintain self-replication capability over many cell divisions (>300). The results described in this article were obtained with cells between 100 and 400 passages. Cells were grown in antibiotic treated complete Neurobasal media containing glucose, L-glutamine, Fetal Bovine Serum (FBS), B-27 supplement, and nerve growth factor (NGF) (100 ng/ml). Differentiation and axonal elongation was induced by addition of forskolin (75 pM) into the culture medium. Within hours following forskolin treatment, more than 90% cells stopped dividing and extended long neurites. Methylglyoxal was administered at a final concentration of 250 pM for 20-24 h post-differentiation of the cells. Podocarpic acid was added at a final concentration of 250 pM and incubated for the same period with or without methylglyoxal. Ethanol was used as a vehicle control. Differential Interference Contrast (DIC) imaging was performed using a Nikon Ti PFS fitted with a Cascade 512B EMCCD camera, Sutter filter wheels, and Xenon light source with constant temperature enclosure and CO₂ regulation at the stage. Neurite outgrowth was quantified by manually measuring the length of a projection from the edge of the cell body; a neurite was defined as a thin projection longer than the diameter of the associated cell body. Area of soma or cell body was measured excluding the neurite projections. Images were processed using Image Analyst MKII software (www.imageanalyst.net/) and quantification was done using Image J software (//imagei.nih.gov/ij/). 75-100 cells selected randomly were considered for quantification under each experimental condition.

High-throughput Drug Screen in C. elegans

Synchronized glod-4 L1 animals were cultured on NGM agar plates seeded with E. coli OP50-1 until L4. Animals were then transferred into individual wells (10 animals per well) of 96-well plates for high-throughput screening (NPL640, TimTec LLC, DE); each well containing 150 pL of 66.7 pM of individual drug (1 pL of 10 mM drug stock in DMSO) in S-medium. Post-transfer into wells, animals were fed OP50-1 bacteria ad libitum while incubating at 20° C. on a rocker for 12 h. DMSO was used as control. Post-incubation with drug or DMSO, animals were transferred from wells onto NGM agar plates seeded with OP50-1 bacteria. Touch assay was performed on individual animal and touch index (TI) was calculated for each compound from the library as mentioned earlier.

Compounds that showed an amelioration of the hypersensitivity phenotype of glod-4 young adult animals in this screen were subjected to a secondary screen for amelioration of the short lifespan phenotype associated with glod-4 animals.

Calcium Imaging of HEK293T Cells

Appropriate HEK293T-derived cells were seeded on collagen-coated glass bottom culture dishes (MatTek Corporation). Cells were loaded with 10 pM of Rhod-3 AM (Life Technology) for 30 min at 37° C. After 30 min they were washed twice with standard Tyrode's solution (135 mM NaCl, 4 mM KCl, 10 mM glucose, 10 mM HEPES, 2 mM CaCl₂, and 1 mM MgCl₂ at pH=7.4) at room temperature. Calcium imaging was performed on an Olympus BX51WI Axiovert microscope under a 60× objective. Fluorescent images were documented upon sequential excitation with 555 nm followed by 484 nm with a Roper CoolSnap CCD camera. After establishing a baseline 555/484 ratio, methylglyoxal or other agonists were diluted with Tyrode's solution (100 pM or 1 mM final concentration) and were perfused into cells. Images were processed with the MetaFlour (Olympus) software.

Statistical Analyses.

All data analyses for lifespan were performed using GraphPad Prism 6 (GraphPad Software, Inc., La Jolla, Calif.). Survival curves were plotted using Kaplan-Meier method and comparison between survival curves to measure significance (P values) was performed using Log-rank (Mantel-Cox) test. All remaining pairwise comparisons for the quantification data were done using two-tailed Student's t-test. P values from the significance testing were designated as follows: *P<0.05, **P<0.005 and ***P<0.0005.

### REFERENCES

An, J. H., and Blackwell, T. K. (2003). SKN-1 links C. elegans mesendodermal specification to a conserved oxidative stress response. Genes & development 17, 1882-1893.

An, J. H., Vranas, K., Lucke, M., Inoue, H., Hisamoto, N., Matsumoto, K., and Blackwell, T. K. (2005). Regulation of the Caenorhabditis elegans oxidative stress defense protein SKN-1 by glycogen synthase kinase-3. Proceedings of the National Academy of Sciences of the United States of America 102, 16275-16280.

Andersson, D. A., Gentry, C., Light, E., Vastani, N., Vallortigara, J., Bierhaus, A., Fleming, T., and Bevan, S. (2013). Methylglyoxal evokes pain by stimulating TRPA1. PloS one 8, e77986.

Barajas, B., Che, N., Yin, F., Rowshanrad, A., Orozco, L. D., Gong, K. W., Wang, X., Castellani, L. W., Reue, K., Lusis, A. J., et al. (2011). NF-E2-related factor 2 promotes atherosclerosis by effects on plasma lipoproteins and cholesterol transport that overshadow antioxidant protection. Arteriosclerosis, thrombosis, and vascular biology 31, 58-66.

Bierhaus, A., Fleming, T., Stoyanov, S., Leffler, A., Babes, A., Neacsu, C., Sauer, S. K., Eberhardt, M., Schnolzer, M., Lasitschka, F., et al. (2012). Methylglyoxal modification of Nav1.8 facilitates nociceptive neuron firing and causes hyperalgesia in diabetic neuropathy. Nature medicine 18, 926-933.

Chen, W., Mi, R., Haughey, N., Oz, M., and Hoke, A. (2007). Immortalization and characterization of a nociceptive dorsal root ganglion sensory neuronal line. Journal of the peripheral nervous system : JPNS 12, 121-130.

Cui, Y. M., Yasutomi, E., Otani, Y., Yoshinaga, T., Ido, K., Sawada, K., and Ohwada, T. (2008). Design, synthesis and characterization of podocarpate derivatives as openers of BK channels. Bioorganic & medicinal chemistry letters 18, 5197-5200.

da Costa, G., Gomes, R. A., Guerreiro, A., Mateus, E., Monteiro, E., Barroso, E., Coelho, A. V., Freire, A. P., and Cordeiro, C. (2011). Beyond genetic factors in familial amyloidotic polyneuropathy: protein glycation and the loss of fibrinogen's chaperone activity. PloS one 6, e24850.

DeNicola, G. M., Karreth, F. A., Humpton, T. J., Gopinathan, A., Wei, C., Frese, K., Mangal, D., Yu, K. H., Yeo, C. J., Calhoun, E. S., et al. (2011). Oncogene-induced Nrf2 transcription promotes ROS detoxification and tumorigenesis. Nature 475, 106-109.

Distler, M. G., and Palmer, A. A. (2012). Role of Glyoxalase 1 (Glo1) and methylglyoxal (MG) in behavior: recent advances and mechanistic insights. Frontiers in genetics 3, 250.

Giacco, F., Du, X., D'Agati, V. D., Milne, R., Sui, G., Geoffrion, M., and Brownlee, M. (2014). Knockdown of glyoxalase 1 mimics diabetic nephropathy in nondiabetic mice. Diabetes 63, 291-299.

Gomes, M. B., and Negrato, C. A. (2014). Alpha-lipoic acid as a pleiotropic compound with potential therapeutic use in diabetes and other chronic diseases. Diabetology & metabolic syndrome 6, 80.

Gomes, R., Sousa Silva, M., Quintas, A., Cordeiro, C., Freire, A., Pereira, P., Martins, A., Monteiro, E., Barroso, E., and Ponces Freire, A. (2005). Argpyrimidine, a methylglyoxal-derived advanced glycation end-product in familial amyloidotic polyneuropathy. The Biochemical journal 385, 339-345.

Henning, C., Liehr, K., Girndt, M., Ulrich, C., and Glomb, M. A. (2014). Extending the spectrum of alpha-dicarbonyl compounds in vivo. The Journal of biological chemistry 289, 28676-28688.

Hertweck, M., Gobel, C., and Baumeister, R. (2004). C. elegans SGK-1 is the critical component in the Akt/PKB kinase complex to control stress response and life span. Developmental cell 6, 577-588.

Hobert, O., Moerman, D. G., Clark, K. A., Beckerle, M. C., and Ruvkun, G. (1999). A conserved LIM protein that affects muscular adherens junction integrity and mechanosensory function in Caenorhabditis elegans. The Journal of cell biology 144, 45-57.

Jimenez-Osorio, A. S., Gonzalez-Reyes, S., and Pedraza-Chaverri, J. (2015). Natural Nrf2 activators in diabetes. Clinica chimica acta; international journal of clinical chemistry 448, 182-192.

Julius, D. (2013). TRP channels and pain. Annual review of cell and developmental biology 29, 355-384.

Kindt, K. S., Viswanath, V., Macpherson, L., Quast, K., Hu, H., Patapoutian, A., and Schafer, W. R. (2007). Caenorhabditis elegans TRPA-1 functions in mechanosensation. Nature neuroscience 10, 568-577.

Lee, J. Y., Song, J., Kwon, K., Jang, S., Kim, C., Baek, K., Kim, J., and Park, C. (2012). Human DJ-1 and its homologs are novel glyoxalases. Human molecular genetics 21, 3215-3225.

Liu, B., and Qin, F. (2005). Functional control of cold- and menthol-sensitive TRPM8 ion channels by phosphatidylinositol 4,5-bisphosphate. The Journal of neuroscience : the official journal of the Society for Neuroscience 25, 1674-1681.

Macpherson, L. J., Dubin, A. E., Evans, M. J., Marr, F., Schultz, P. G., Cravatt, B. F., and Patapoutian, A. (2007). Noxious compounds activate TRPA1 ion channels through covalent modification of cysteines. Nature 445, 541-545.

Morcos, M., Du, X., Pfisterer, F., Hutter, H., Sayed, A. A., Thornalley, P., Ahmed, N., Baynes, J., Thorpe, S., Kukudov, G., et al. (2008). Glyoxalase-1 prevents mitochondrial protein modification and enhances lifespan in Caenorhabditis elegans. Aging cell 7, 260-269.

More, S. S., Vartak, A. P., and Vince, R. (2013). Restoration of glyoxalase enzyme activity precludes cognitive dysfunction in a mouse model of Alzheimer's disease. ACS chemical neuroscience 4, 330-338.

Pandey, U. B., and Nichols, C. D. (2011). Human disease models in Drosophila melanogaster and the role of the fly in therapeutic drug discovery. Pharmacological reviews 63, 411-436.

Petrascheck, M., Ye, X., and Buck, L. B. (2007). An antidepressant that extends lifespan in adult Caenorhabditis elegans. Nature 450, 553-556.

Rabbani, N., and Thornalley, P. J. (2011). Glyoxalase in diabetes, obesity and related disorders. Seminars in cell & developmental biology 22, 309-317.

Rabbani, N., and Thornalley, P. J. (2014). Measurement of methylglyoxal by stable isotopic dilution analysis LC-MS/MS with corroborative prediction in physiological samples. Nature protocols 9, 1969-1979.

Reiner, D. J., Newton, E. M., Tian, H., and Thomas, J. H. (1999). Diverse behavioural defects caused by mutations in Caenorhabditis elegans unc-43 CaM kinase II. Nature 402, 199-203.

Riddle, D. L., Blumenthal, T., Meyer, B. J., and Priess, J. R. (1997). Introduction to C. elegans . In C elegans II, D. L. Riddle, T. Blumenthal, B. J. Meyer, and J. R. Priess, eds. (Cold Spring Harbor (NY)).

Robatzek, M., and Thomas, J. H. (2000). Calcium/calmodulin-dependent protein kinase II regulates Caenorhabditis elegans locomotion in concert with a G(o)/G(q) signaling network. Genetics 156, 1069-1082.

Santiago, J. A., and Potashkin, J. A. (2014). System-based approaches to decode the molecular links in Parkinson's disease and diabetes. Neurobiology of disease 72 Pt A, 84-91.

Sporn, M. B., and Liby, K. T. (2012). NRF2 and cancer: the good, the bad and the importance of context. Nature reviews Cancer 12, 564-571.

Staab, T. A., Griffen, T. C., Corcoran, C., Evgrafov, O., Knowles, J. A., and Sieburth, D. (2013). The conserved SKN-1/Nrf2 stress response pathway regulates synaptic function in Caenorhabditis elegans. PLoS genetics 9, e1003354.

Stiernagle, T. (2006). Maintenance of C. elegans . WormBook, 1-11.

Suzuki, T., Motohashi, H., and Yamamoto, M. (2013). Toward clinical application of the Keapl-Nrf2 pathway. Trends in pharmacological sciences 34, 340-346.

Takekuma, K., Ando, F., Niino, N., and Shimokata, H. (2002). Prevalence of hyperesthesia detected by current perception threshold test in subjects with glucose metabolic impairments in a community. Internal medicine 41, 1124-1129.

Teichert, J., Hermann, R., Ruus, P., and Preiss, R. (2003). Plasma kinetics, metabolism, and urinary excretion of alpha-lipoic acid following oral administration in healthy volunteers. Journal of clinical pharmacology 43, 1257-1267.

Toyoda, Y., Erkut, C., Pan-Montojo, F., Boland, S., Stewart, M. P., Muller, D. J., Wurst, W., Hyman, A. A., and Kurzchalia, T. V. (2014). Products of the Parkinson's disease-related glyoxalase DJ-1, D- lactate and glycolate, support mitochondrial membrane potential and neuronal survival. Biology open 3, 777-784.

Vallianou, N., Evangelopoulos, A., and Koutalas, P. (2009). Alpha-lipoic Acid and diabetic neuropathy. The review of diabetic studies: RDS 6, 230-236.

Wang, J., Robida-Stubbs, S., Tullet, J. M., Rual, J. F., Vidal, M., and Blackwell, T. K. (2010). RNAi screening implicates a SKN-1-dependent transcriptional response in stress resistance and longevity deriving from translation inhibition. PLoS genetics 6.

Wei, H., Hamalainen, M. M., Saarnilehto, M., Koivisto, A., and Pertovaara, A. (2009). Attenuation of mechanical hypersensitivity by an antagonist of the TRPA1 ion channel in diabetic animals. Anesthesiology 111, 147-154.

Xiao, R., Zhang, B., Dong, Y., Gong, J., Xu, T., Liu, J., and Xu, X. Z. (2013). A genetic program promotes C. elegans longevity at cold temperatures via a thermosensitive TRP channel. Cell 152, 806-817.

Xue, M., Rabbani, N., Momiji, H., Imbasi, P., Anwar, M. M., Kitteringham, N., Park, B. K., Souma, T., Moriguchi, T., Yamamoto, M., et al. (2012). Transcriptional control of glyoxalase 1 by Nrf2 provides a stress-responsive defence against dicarbonyl glycation. The Biochemical journal 443, 213-222.

Yang, Y., Chen, G., Cheng, X., Teng, Z., Cai, X., Yang, J., Sun, X., Lu, W., Wang, X., Yao, Y., et al. (2015). Therapeutic potential of digitoflavone on diabetic nephropathy: nuclear factor erythroid 2- related factor 2-dependent anti-oxidant and anti-inflammatory effect. Scientific reports 5, 12377.

### Supplement—Extended Experimental Procedures

Strains

Nematode stocks were maintained on Nematode Growth Medium (NGM) plates made with Bacto agar (BD Biosciences) and seeded with bacteria (Escherichia coli strain OP50-1 unless otherwise specified) at 20° C. (http://www.wormbook.org/). The following C. elegans strains were used: wild type (N2, Bristol), VC343: glod-4(gk189), BC15643: sEx15643 [rCesC16C10.10::gfp+pCeh361], OH438: otIs117 [unc-4(+)+unc-33p::gfp], CL2166: dvIs19 [(pAF15)gst-4p::gfp::nls], LD1171: Idls3 [gcs-1p::gfp+pRF4(rol-6(su1006))], EU31: skn-1(zu135) IV/nT1 [unc-?(n754) let-?], LG348: skn-1(zu135)/nT1[qIs51], gels9[gpa-4p:: skn-1b::gfp+rol-6(su1006)], LG357: skn-1(zu135)/nT1[qIs51], gels10[ges-1p::skn-1c::gfp+rol-6(su1006)], LD1:Idls7 [skn-1b/c::gfp+pRF4(rol-6(su1006))], KU25: pmk-1(km25), KU4: sek-1(km4), VC345: sgk-1(ok538), CX10: osm-9(ky10), CX4544: ocr-2(ak47), TQ225: trp-1(sy690), TQ194: trp-2(sy691), TQ296: trp-4(sy695), TQ233: trpa-1(ok999), TQ1643: XuEx601[ges-1p::trpa-1::s12::yfp+unc-122p::dsred], TQ1648: XuEx606[rgef-1p::trpa-1::s12::yfp+unc-122p::dsred], TQ1657: XuEx610[myo-3p::trpa-1::s12::yfp+unc-122p::dsred], TQ1658: XuEx611[dpy- 7p::trpa-1::s12::yfp+unc-122p::dsred], TQ2772: XuEx866[pges-1::trpa-1(E1018A)::s12::mcherry2];XuEx19[plfe-2b::GCaMP1.3+plfe-2b::dsred], trpa-1(ok999), TQ1996: unc-43(n498, n1186), TQ2746: cmk-1(oy21), TQ2571:pkc-2(ok328), TQ3056: XuIs180[plfe2b::GCaMP1.3+plfe-2b::dsred];N2, TJ356: zIs356[daf-16p::daf-16a/b::GFP+rol-6], BC15643: sEx15643[rCes C16C10. 10::gfp+pCeh361], glod-4;dvIs19 [(pAF15)gst-4p::gfp::nls], glod-4; otIs117 [unc-4(+)+unc-33p::gfp]. Compound mutants were constructed using standard techniques. Bacterial clones for RNAi feeding protocol were obtained from either the Ahringer library (Kamath and Ahringer (2003) Methods, 30: 313-321) or the ORFeome RNAi v1.1 library (Open Biosystems, GE Healthcare, CO).

Glod-4p::gfp Expression Assay

Transgenic adult animals carrying glod-4p::gfp were assayed. Fluorescent intensity was calculated by background subtraction and quantified for the same region of interest across individual animals. All quantification was done using Image J™ software (//imagei.nih.gov/ii/).

Swim-bend Assay for Health-span Assessment

Motility is a determinant of healthspan and rhythmic behavioral patterns as observed in C. elegans crawling versus swimming are direct functions of neuromuscular activity (Pierce-Shimomura et al. (2008) Proc. Natl. Acad. Sci. USA, 105: 20982-20987). C. elegans lateral swimming movements was measured as previously described as ‘thrashing’ (Hart (2006) Behavior, WormBook; Pierce-Shimomura et al. (2008) Proc. Natl. Acad. Sci. USA, 105: 20982-20987) by scoring number of body bends per 30 seconds in S-medium. This gave a direct measure of the swim bend frequency and hence a relative measure of healthspan of the animal under different conditions.

Analytical Instrumentation and Software

High performance liquid chromatography (HPLC) was performed using a Shimadzu UFLC prominence system fitted with following modules: CBM-20A (Communication bus module), DGU-A₃ (degasser), two LC-20AD (liquid chromatograph, binary pump), SIL-20AC HT (auto sampler) and connected to a Phenomenex's Kinetex® EVO C18 column (2.1×150 mm, 5 pm, 100 A). Mass spectrometry (MS) was performed using a 4000 QTRAP® LC-MS/MS mass spectrometer from AB SCIEX fitted with a Turbo V™ ion source. AB SCIEX'S ANALYST® v1.6.1 was used for all forms of data acquisition, development of HPLC method, and optimization of analyte-specific MRM (multiple reaction monitoring) transitions. AB SCIEX'S PIEKVIEW® v2.1 and SKYLINE® v3.5 MacLean et al. (2010) Bioinformatics, 26: 966-968) was used for LC-MS/MS data analysis.

Preparation of Metabolome Extracts and Synthetic Standards

Worms were cultured on NGM agar plates as explained before for lifespan experiments with some modifications: ˜100 animals/60 mm plates were used for each experimental replicate and animals were harvested at day 4 of adulthood with 20 pL M9 buffer in 1.5 mL eppendorf tubes. Worm suspensions were flash-frozen over liquid nitrogen and subsequently homogenized ultrasonically using a Fisher Scientific's 550 Sonic Dismembrator with 80 pL of sodium formate buffer (pH=3) containing 75 pM of 2,3-hexanedione (internal standard, IS). Two 20 s pulses at amplitude setting 4 of the instrument (on ice) were sufficient to completely homogenize worm bodies. To each homogenate tube 20 pL of 100 mg/mL o-phenelynediamine (OPD) solution in sodium formate buffer (pH=3) were added, and the mixture was allowed to react in dark at room temperature for ˜22 h. The long reaction time and the pH are critical parameters in this protocol: first to ensure complete derivatization of a-DCs bound reversibly to protein/glutathione —SHs; and second to prevent in vitro a-DC generation from glycolytic intermediates (DHAP, GAP, etc.) or due to DNA breakdown (Chaplen et al. (1998) Proc. Natl. Acad. Sci. USA, 95: 5533-5538). At the end of the stipulated reaction window, 12 pL of 5 M perchloric acid was added to each tube and incubated on ice for 30 min to ensure complete protein precipitation. Subsequently, the tubes were centrifuged at 10,000 rpm for 10 min, and the supernatant collected and neutralized with 30 pL 4 M NH₄OH solution. All glod-4-derived samples (except with podocarpic acid or a-lipoic acid treated animals) were diluted 50-fold with methanol and 1 pL of each sample injected for LC-MS/MS analysis; all other samples were injected (1 pL) without dilution.

Synthetic standards for glyoxal, methylglyoxal, and 3-deoxyglucosone were obtained from Sigma-Aldrich, St. Louis, Mo. Corresponding OPD derivatives were prepared as mentioned before (with IS), starting with 20 pL solution of each of these compounds in M9 at the following concentrations: 5 mM, 500 pM, 50 pM, and 5 pM. As previously noted (Henning et al. (2014) J. Biol. Chem., 289: 28676-28688), almost quantitative derivatization was achieved after ˜12 h of reaction for synthetic standards. Each of these samples were diluted 50-fold with methanol at the end to achieve individual 100 pM, 10 pM, 1 pM, and 100 nM solutions, 1 pL of which were injected for LC-MS/MS analysis. For every batch of worm samples analysis, a set of synthetic standards were processed.

MRM Optimization, LC-MS/MS Conditions, and Data Analyses

Optimization of analyte-specific MRM transitions, such as determination of suitable precursor and product ions and optimal MS parameters for each transition (Q1, precursor→Q₃, product) were achieved by isocratic flow injection of the 10 pM solution (final) for each standard- or IS-OPD derivatives. The most intense (Q₁→Q₃) transition was used as quantifier, whereas the next best transition was used as qualifier for each compound (Table 4). For LC separation, a solvent gradient of 0.1% acetic acid in water (aqueous) - methanol (organic) was used with 0.4 mL/min flow rate, starting with an acetonitrile content of 3% for 0.7 min, which was increased to 100% over 6 min and held at 100% for 1.5 min. The LC column was subsequently reconstituted to its initial condition (methanol content of 3%) over the next 0.5 min and re-equilibrated for 3 min.

Derivatized metabolome extracts, as well as synthetic a-DCs were analyzed by scheduled LC-MRM in positive ion mode. To develop the scheduled LC-MRM method, MS/MS data was collected for all transitions across the length of each LC run for a mixture of synthetic GO-, MGO-, 3DG-, and 2,3-hexanedione (IS)-OPD derivatives (1 pL injection of 1 pM final concentration). Source conditions were as follows: curtain gas (CUR) 20, nebulizer gas (GS1) 60, auxiliary gas (GS2) 50, ionspray voltage (IS) 4500 V, and source temperature (TEM) 450° C. This step was undertaken to ascertain the LC retention times (RT) for the OPD derivatives. Several such sets were acquired to compute analyte-specific variability in RTs. Next, the MS was switched to operate in scheduled MRM mode, whereby the mass spectrometer acquired data for specific MRM transitions ±45 s around the computed RT for the analyte (Table 4). Relative quantification of GO, MGO, and 3DG were based on integration of corresponding OPD derivative-specific quantifier peaks obtained from scheduled LC-MRM runs (peak areas) and adjusted to the number of animals. To account for OPD derivatization efficiencies in individual tubes, sample-to-sample variability in MS response, and differential sample dilutions (both for synthetic a-DCs as well as a-DCs in worm homogenates), the peak areas were normalized to the quantifier peak area for IS-OPD for each sample.

SKN-1 Activation Assay

Adult animals were examined for SKN-1/Nrf2 activation using GFP reporters for both SKN-1 and SKN-1 target genes: gst-4 and gcs-1. For exogenous MGO and drug assays, age-synchronized GFP reporter strains were subject to control and treatment conditions for a period of 4-6 hours before microscopy. For RNAi-induced activation studies, GFP reporter strains were fed on HT1115 bacteria expressing empty vector pL4440 or RNAi gene from L1 stage. Synchronized day 1 adult animals were then subjected to microscopic examination. Activation of SKN-1 using SKN-1 fusion reporter was determined as described earlier (An et al. (2003) Genes & Dev. 17: 1882-1893; Onken and Driscoll (2010) PloS One, 5: e8758). Scoring for downstream SKN-1 targets carrying GFP reporter for gcs-1 promoter was performed based on a previous study (Wang et al. (2010) PLoS Genetics, doi. org/10.1371/journal.pgen.1001048) and categorized as follows: ‘high’ for strong GFP signal throughout the intestine, ‘medium’ for GFP signal in the anterior or posterior section of the intestine and ‘low’ for weak or no signal. For animals with gst-4 promoters carrying GFP reporters number of GST-4 positive foci was counted under different conditions as described previously (Fensgardet al. (2010) Aging, 2: 133-159). Quantification of acquired images was done using the Image J™ software (//imagej.nih.gov/ij/).

Ca+² Flux-based channel studies in C. elegans

Ca²⁺ flux was measured using an upright Olympus compound microscope (BX51) under a 40× objective. Real time sequential G-CaMP1.3 fluorescent images were captured using Hamamatsu Ocra-ER digital CCD camera at a frame rate of 10 frames per second and for a span of 300 frames. Acquired images were analyzed for further intensity measurements using HC Image software v. 1.1.3.0 (Hamamatsu Corp., NJ). Percentage change in fluorescent peak intensity was estimated using the intensity values generated real time by the program.

Reverse Transcription Polymerase Chain Reaction (RT-PCR)

Total RNA was extracted from nearly 100 age-synchronized adult animals picked and collected in 20 pl of M9 buffer using TRIzol reagent (Life Technologies, CA). Subsequently, 1 pg total RNA was used as template for cDNA synthesis. cDNA was synthesized using the ISCRIPT™ cDNA synthesis kit (Bio-Rad, CA) following manufacturer's protocol. qRT-PCR was carried out using the SensiFAST SYBR No-ROX kit (Bioline, MA) in a LightCycler 480 Real-Time PCR system (Roche Diagnostics Corp., IN). Quantification was performed using the comparative AACt method and normalization for internal reference was done using actin gene act-1.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method for the treatment or prophylaxis of diabetes in a mammal, said method comprising:
administering to a mammal identified as having diabetes or pre-diabetes an agent that activates TRPA1 in an amount sufficient to ameliorate one or more symptoms of diabetes or pre-diabetes.

2. The method of claim 1, wherein said amount sufficient to ameliorate one or more symptoms of diabetes or pre-diabetes is an amount sufficient to ameliorate a complication of diabetes selected from the group consisting of diabetic neuropathy, cardiomyopathy, nephropathy, retinopathy, microvascular damage, and early mortality.

3. A method of ameliorating a pathology characterized by elevated α-dicarbonyl compounds and advanced glycation endproducts or prophylactically slowing or stopping the onset of said pathology in a mammal, said method comprising:
administering to said mammal an agent that activates TRPA1 in an amount sufficient to activate TRPA1 and/or to ameliorate one or more symptoms of said pathology, and/or to slow or stop the onset of said pathology, and/or to lower the level of dicarbonyl compounds in said mammal.

4. The method of claim 3, wherein said pathology is selected from the group consisting of Diabetes, Alzheimer's disease, Parkinson's disease, ATTR amyloidosis, cataract formation, stroke, and cardiovascular disease.

5. The method of claim 3, wherein said pathology is diabetes.

6. The method of claim 3, wherein said pathology is hyperglycemia.

7. A method of reducing the levels of α-dicarbonyl compounds and advanced glycation endproducts in a mammal, said method comprising:
administering to said mammal an agent that activates TRPA1 in an amount sufficient to lower the level of α-dicarbonyl compounds and advanced glycation endproducts in said mammal.

8. A method of reducing a method of reducing the amount of, or slowing or stopping the formation and/or accumulation of, advanced glycation endproducts in a mammal, said method comprising:
administering to said mammal an agent that activates TRPA1 in an amount sufficient to slow or stop the accumulation of advanced glycation endproducts in said mammal.

9. The method of claim 1, wherein said mammal is a mammal identified as having elevated triglycerides.

10. The method of claim 1, wherein said mammal is a mammal diagnosed as pre-diabetic.

11. The method of claim 1, wherein said mammal is a mammal diagnosed as having diabetes.

12. The method of claim 1, wherein said method produces a reduction in one or more advanced glycation endproducts.

13. The method of claim 12, wherein said method produces a reduction in, or slows the accumulation of, glyoxal/GO.

14. The method of claim 12, wherein said method produces a reduction in, or slows the accumulation of, methylglyoxal/MGO.

15. The method of claim 12, wherein said method produces a reduction in, or slows the accumulation of 3-deoxyglucosone/3DG.

16. The method of claim 1, wherein said mammal is a human.

17. (canceled)

18. The method of claim 1, wherein said TRPA1 activator is not a natural product other than podocarpic acid and/or a podocarpic acid derivative.

19. The method of claim 1, wherein method does not involve administering an agent selected from the group consisting of vitamin C, benfotiamine, pyridoxamine, alpha-lipoic acid, taurine, pimagedine, aspirin, carnosine, metformin, pioglitazone, pentoxifylline, resveratrol, and curcumin.

20. The method of claim 1, wherein said TRPA1 activator comprises podocarpic acid or an analog and/or derivative thereof or a pharmaceutically acceptable salt of said podocarpic acid or analog and/or derivative thereof.

21. The method of claim 20, wherein said podocarpic analog or derivative comprises podocarpanol or a pharmaceutically acceptable salt thereof.

22. The method of claim 20, wherein said podocarpic analog or derivative comprises a compound selected from the compounds shown in Table 1, Table 2, or Table 3 or a pharmaceutically acceptable salt thereof.

23. The method of claim 1, wherein said TRPA1 activator comprises an indolinone compound according to formula I or a pharmaceutically acceptable salt thereof.

24. The method of claim 21, wherein said indolinone compound is selected from the group consisting of is (2E)[1-(cyclohexylmethyl)-2-oxo-1,2-dihydro-3H-indol-3-ylidene]acetic acid, (2E)-(1-benzyl-5-fluoro-2-oxo-1,2-dihydro-3H-indol-3-ylidene)acetic acid, (2E)-(1-benzyl-7-fluoro-2-oxo-1,2-dihydro-3H-indol-3-ylidene)acetic acid, (2E)-[-(cyclopentylmethyl)-2-oxo-1,2-dihydro-3H-indol-3-ylidene]acetic acid, (2E)-(7-fluoro-1-isobutyl-2-oxo-1,2-dihydro-3H-indol-3-ylidene)acetic acid, (2E)-[1-(cyclopentylmethyl)-7-fluoro-2-oxo-1,2-dihydro-3H-indol-3-ylidene]acetic acid, (2E)-(7-chloro-1-isobutyl-2-oxo-1,2-dihydro-3H-indol-3-ylidene)acetic acid, (2E)-[-(cyclobutylmethyl)-7-fluoro-2-oxo-1,2-dihydro-3H-indol-3-ylidene]acetic acid, (2E)-[1-(cyclopropylmethyl)-7-fluoro-2-oxo-1,2-dihydro-3H-indol-3-ylidene]acetic acid, (2E)-2-[1-(cyclopentylmethyl)-7-fluoro-2-oxo-1,2-dihydro-3H-indol-3-ylidene]-N,N-dimethylacetamide, (3E)-1-(2-ethyl butyl)-7-fluoro-3-(2-morpholin-4-yl-2-oxoethylidene)-1,3-dihydro-2H-indol-2-one, (2E)-{7-fluoro-1-[(2S)-2-methylbutyl]-2-oxo-1,2-dihydro-3H-indol-3-ylidene}acetic acid, (2E)-[7-fluoro-1-(3-methylbutyl)-2-oxo-1,2-dihydro-3H-indol-3-ylidene]acetic acid, (2E)-2[1-(cyclohexylmethyl)-2-oxo-1,2-dihydro-3H-indol-3-ylidene]-N,N-dimethylacetamide, (2E)-2-[1-(cyclopentylmethyl)-2-oxo-1,2-dihydro-3H-indol-3-ylidene]-N,N-dimethylacetamide, (3E)-3-(2-azetidin-1-yl-2-oxoethylidene)-1-(cyclohexylmethyl)-1,3-dihydro-2H-indol-2-one, (3E)-3-(2-azetidin-1-yl-2-oxoethylidene)-1-(cyclopentylmethyl)-1,3-dihydro-2H-indol-2-one, (2E)-[1-(2-ethylbutyl)-2-oxo-1,2-dihydro-3H-indol-3-ylidene]acetic acid, (3E)-1-(2-ethylbutyl)-3-(2-oxo-2-pyrrolidin-1-ylethylidene)-1,3-dihydro-2-H-indol-2-one, (3E)-3-(2-azetidin-1-yl-2-oxoethylidene)-1-(2-ethylbutyl)-1,3-dihydro-2H-indol-2-one, (3E)-3-(2-azetidin-1-yl-2-oxoethylidene)-1-(cyclobutylmethyl)-1,3-dihydro-2H-indol-2-one, and (3E)-1-(cyclobutylmethyl)-3-(2-oxo-2-pyrrolidin-1-ylethylidene)-1,3-dihydro-2H-indol-2-one.

25. The method according of claim 20, wherein said compound is a substantially pure enantiomer.
